SEQUENCE LISTING (56 sequences)

SEQ ID NO: 1 - protein, 526 aa, Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 2 - protein, 526 aa, Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKYIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 3 - protein, 387 aa, Plasmodium falciparum
KNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 4 - protein, 387 aa, Plasmodium falciparum
KNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 5 - protein, 367 aa, Plasmodium falciparum
SIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 6 - protein, 367 aa, Plasmodium falciparum
SIDILQEKEGHLDFVIIPHYTFLDYYKHLSYNSIYHKSSTYGKYIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 7 - protein, 338 aa, Artificial Sequence
  PfRH5 (3D7) sequence excluding flexible N-terminal (1-139) and flexible loop regions
KNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 8 - protein, 338 aa, Artificial Sequence
  PfRH5 (7G8) sequence excluding flexible N-terminal (1-139) and flexible loop regions
KNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 9 - protein, 318 aa, Artificial Sequence
  PfRH5 (3D7) sequence excluding flexible N-terminal (1-159) and flexible loop regions
SIDILQEKEGHLDFVIIPHYTFLDYYKHLSYNSIYHKSSTYGKCIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 10 - protein, 318 aa, Artificial Sequence
  PfRH5 (7G8) sequence excluding flexible N-terminal (1-159) and flexible loop regions
SIDILQEKEGHLDFVIIPHYTFLDYYKHLSYNSIYHKSSTYGKYIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 11 - protein, 411 aa, Artificial Sequence
  Sv2 (3D7) sequence excluding flexible N-terminal (1-139) region, with Bip leader and Hexa-His tag
MKLCILLAVVAFVGLSLGKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQHHHHHH

SEQ ID NO: 12 - protein, 411 aa, Artificial Sequence
  Sv2 (7G8) sequence excluding flexible N-terminal (1-139) region, with Bip leader and Hexa-His tag
MKLCILLAVVAFVGLSLGKNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQHHHHHH

SEQ ID NO: 13 - protein, 362 aa, Artificial Sequence
  Sv3 (3D7) sequence excluding flexible N-terminal (1-139) and flexible loop regions, with Bip leader and Hexa-His tag
MKLCILLAVVAFVGLSLGKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQHHHHHH

SEQ ID NO: 14 - protein, 362 aa, Artificial Sequence
  Sv3 (7G8) sequence excluding flexible N-terminal (1-139) and flexible loop regions, with Bip leader and Hexa-His tag
MKLCILLAVVAFVGLSLGKNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNKNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQHHHHHH

SEQ ID NO: 15 - protein, 526 aa, Artificial Sequence
  Modified full length PfRH5 amino acid sequence (3D7) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNLANSIDILQEKEGHLDFVIIPHYTFLEYYKHLSYNSIYHKSSTYGKCIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 16 - protein, 526 aa, Artificial Sequence
  Modified full length PfRH5 amino acid sequence (7G8) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNLANSIDILQEKEGHLDFVIIPHYTFLEYYKHLSYNSIYHKSSTYGKYIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 17 - protein, 387 aa, Artificial Sequence
  Modified PfRH5 amino acid sequence (3D7) excluding signal sequence and flexible N-terminal region
KNVNFLQYHFKELSNYNLANSIDILQEKEGHLDFVIIPHYTFLEYYKHLSYNSIYHKSSTYGKCIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 18 - protein, 387 aa, Artificial Sequence
  Modified PfRH5 amino acid sequence (7G8) excluding signal sequence and flexible N-terminal region
KNVNFLQYHFKELSNYNLANSIDILQEKEGHLDFVIIPHYTFLEYYKHLSYNSIYHKSSTYGKYIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 19 - protein, 367 aa, Artificial Sequence
  Modified PfRH5 amino acid sequence (3D7) excluding signal sequence and flexible N-terminal region
SIDILQEKEGHLDFVIIPHYTFLEYYKHLSYNSIYHKSSTYGKCIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 20 - protein, 367 aa, Artificial Sequence
  Modified PfRH5 amino acid sequence (7G8) excluding signal sequence and flexible N-terminal region
SIDILQEKEGHLDFVIIPHYTFLEYYKHLSYNSIYHKSSTYGKYIAVDAFIKKINETYDKVKSKCNDIKNDLIKTIKKLEHPYDINNKNDDSYRYDISEEIDDKSEETDDETEEVEDSIQDTDSNHTPSNKKKNDLMNRTFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 21 - protein, 338 aa, Artificial Sequence
  HS1 excluding signal sequence, flexible N-terminal and flexible loop
KNVNFLQYHFKELSNYNLANSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 22 - protein, 318 aa, Artificial Sequence
  HS1 excluding signal sequence, flexible N-terminal and flexible loop
SIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 23 - protein, 344 aa, Artificial Sequence
  HS1 excluding signal sequence, flexible N-terminaland flexible loop and comprising a Hexa-histidine C-terminal tag
KNVNFLQYHFKELSNYNLANSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQHHHHHH

SEQ ID NO: 24 - protein, 324 aa, Artificial Sequence
  HS1 excluding signal sequence, flexible N-terminal and flexible loop and comprising a Hexa-histidine C-terminal tag
SIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQHHHHHH

SEQ ID NO: 25 - protein, 356 aa, Artificial Sequence
  HS1 excluding signal sequence, flexible N-terminal and flexible loop and comprising a Bip leader sequence
MKLCILLAVVAFVGLSLGKNVNFLQYHFKELSNYNLANSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 26 - protein, 336 aa, Artificial Sequence
  HS1 excluding signal sequence, flexible N-terminal and flexible loop and comprising a Bip leader sequence
MKLCILLAVVAFVGLSLGSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQ

SEQ ID NO: 27 - protein, 362 aa, Artificial Sequence
  HS1 excluding signal sequence, flexible N-terminal and flexible loop and comprising a Bip leader sequence and a Hexa-histidine C-terminal tag
MKLCILLAVVAFVGLSLGKNVNFLQYHFKELSNYNLANSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQHHHHHH

SEQ ID NO: 28 - protein, 342 aa, Artificial Sequence
  HS1 excluding signal sequence, flexible N-terminal and flexible loop and comprising a Bip leader sequence and a Hexa-histidine C-terminal tag
MKLCILLAVVAFVGLSLGSIDILQEKEGHLDFVIIPHYTFLEYYKHLSYNSIYHKSSTYGKYIAVDAFIKKINEAYDKVKSKCNDIKNDLIKTIKKLEHPYDINNKNRAFKKMFDEYNTKKNKFINCIKNHENDFNKICNDMKNYGTNLFEQLSCYNNNFCNTNGIRYHYDEYIHKLILAVKSKNLNKDLNDMKNILQQSEKLLNNLEKKMGSYIYIDTIKFIHKEMKHIFNRIEYHTKIINDKTKIIQDKIKLNIWRTFQKDELLKKILDMSKEYALFITSDHLRQMLYNTFYSKEKHLNNIFHHLIYVLQMKLNDVPIKMEYFQTYKKNKPLTQHHHHHH

SEQ ID NO: 29 - protein, 526 aa, Artificial Sequence
  Modified full length PfRH5 amino acid sequence (3D7) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLEYYKHLSYIAIYHKSSTYGKCIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 30 - protein, 526 aa, Artificial Sequence
  Modified full length PfRH5 amino acid sequence (7G8) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLEYYKHLSYIAIYHKSSTYGKYIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 31 - protein, 387 aa, Artificial Sequence
  Modified PfRH5 amino acid sequence (3D7) excluding signal sequence and flexible N-terminal region
KNVNFLQYHFKELSNYNIANSIDILQEKEGHLDFVIIPHYTFLEYYKHLSYIAIYHKSSTYGKCIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 32 - protein, 387 aa, Artificial Sequence
  Modified PfRH5 amino acid sequence (7G8) excluding signal sequence and flexible N-terminal region
KNVNFLQYHFKELSNYNIANSIDILQEKEGHLDFVIIPHYTFLEYYKHLSYIAIYHKSSTYGKYIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 33 - protein, 367 aa, Artificial Sequence
  Modified PfRH5 amino acid sequence (3D7) excluding signal sequence and flexible N-terminal region
SIDILQEKEGHLDFVIIPHYTFLEYYKHLSYIAIYHKSSTYGKCIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 34 - protein, 367 aa, Artificial Sequence
  Modified PfRH5 amino acid sequence (7G8) excluding signal sequence and flexible N-terminal region
SIDILQEKEGHLDFVIIPHYTFLEYYKHLSYIAIYHKSSTYGKYIAVDAFIKKINETYDKVKSKCNDIKNDLINTIHKLEHPYDINNKNDDSYRYDISEEIDDKSEETDDETEEVEDSIQDTDSNHTPSNKKKNDLMNRTFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 35 - protein, 338 aa, Artificial Sequence
  HS2 excluding signal sequence, flexible N-terminal and flexible loop
KNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 36 - protein, 318 aa, Artificial Sequence
  HS2 excluding signal sequence, flexible N-terminal and flexible loop
SIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 37 - protein, 344 aa, Artificial Sequence
  HS2 excluding signal sequence, flexible N-terminal and flexible loop and comprising a Hexa-histidine C-terminal tag
KNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQHHHHHH

SEQ ID NO: 38 - protein, 324 aa, Artificial Sequence
  HS2 excluding signal sequence, flexible N-terminal and flexible loop and comprising a Hexa-histidine C-terminal tag
SIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQHHHHHH

SEQ ID NO: 39 - protein, 356 aa, Artificial Sequence
  HS2 excluding signal sequence, flexible N-terminal and flexible loop and comprising a Bip leader sequence
MKLCILLAVVAFVGLSLGKNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 40 - protein, 336 aa, Artificial Sequence
  HS2 excluding signal sequence, flexible N-terminal and flexible loop and comprising a Bip leader sequence
MKLCILLAVVAFVGLSLGSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQ

SEQ ID NO: 41 - protein, 362 aa, Artificial Sequence
  HS2 excluding signal sequence, flexible N-terminal and flexible loop and comprising a Bip leader sequence and a Hexa-histidine C-terminal tag
MKLCILLAVVAFVGLSLGKNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQHHHHHH

SEQ ID NO: 42 - protein, 342 aa, Artificial Sequence
  HS2 excluding signal sequence, flexible N-terminal and flexible loop and comprising a Bip leader sequence and a Hexa-histidine C-terminal tag
MKLCILLAVVAFVGLSLGSIDILQEKEGHLDFVIIPHYTFLEYYKHLSYIAIYHKSSTYGKYIAVDAFIKKINEAYDKVKSKCNDIKNDLINTIHKLEHPYDINNKNRAFKKMMDEYKTKKKKFIKCIKNHENDFNKICMDMKNYGTNLFEQLSCYNNNFCNTNGIRYHYDEYIHKLINKVKSKNLNKDLNDMKNILQQSEDLLRNLKKKMGSYIYIDTIKFILKEMKHIFNRIEYHTKIINDKTKIIQDKIKEDIWRTFQKDELLKRILDMAKEYALFRTSDDLRQMLYNTFYSKEKHLNNIFNHLIYVLQMKLNDVPIPMEYFQTYKKNKPLTQHHHHHH

SEQ ID NO: 43 - protein, 526 aa, Artificial Sequence
  Modified full length PfRH5 amino acid sequence (3D7) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLEYYKHLSYIAIYHKSSTYGKCIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 44 - protein, 526 aa, Artificial Sequence
  Modified full length PfRH5 amino acid sequence (7G8) including signal sequence
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLEYYKHLSYIAIYHKSSTYGKYIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 45 - protein, 387 aa, Artificial Sequence
  Modified PfRH5 amino acid sequence (3D7) excluding signal sequence and flexible N-terminal region
KNVNFLQYHFKELSNYNIANSIDILQEKEGHLDFVIIPHYTFLEYYKHLSYIAIYHKSSTYGKCIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 46 - protein, 387 aa, Artificial Sequence
  Modified PfRH5 amino acid sequence (7G8) excluding signal sequence and flexible N-terminal region
KNVNFLQYHFKELSNYNIANSIDILQEKEGHLDFVIIPHYTFLEYYKHLSYIAIYHKSSTYGKYIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 47 - protein, 367 aa, Artificial Sequence
  Modified PfRH5 amino acid sequence (3D7) excluding signal sequence and flexible N-terminal region
SIDILQEKEGHLDFVIIPHYTFLEYYKHLSYIAIYHKSSTYGKCIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 48 - protein, 367 aa, Artificial Sequence
  Modified PfRH5 amino acid sequence (7G8) excluding signal sequence and flexible N-terminal region
SIDILQEKEGHLDFVIIPHYTFLEYYKHLSYIAIYHKSSTYGKYIAVDAFIKKINETYDKVKSKCNDIKNDLINTIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 49 - protein, 338 aa, Artificial Sequence
  HS3 excluding signal sequence, flexible N-terminal and flexible loop
KNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 50 - protein, 318 aa, Artificial Sequence
  HS3 excluding signal sequence, flexible N-terminal and flexible loop
SIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 51 - protein, 344 aa, Artificial Sequence
  HS3 excluding signal sequence, flexible N-terminal and flexible loop and comprising a Hexa-histidine C-terminal tag
KNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQHHHHHH

SEQ ID NO: 52 - protein, 324 aa, Artificial Sequence
  HS3 excluding signal sequence, flexible N-terminal and flexible loop and comprising a Hexa-histidine C-terminal tag
SIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQHHHHHH

SEQ ID NO: 53 - protein, 356 aa, Artificial Sequence
  HS3 excluding signal sequence, flexible N-terminal and flexible loop and comprising a Bip leader sequence
MKLCILLAVVAFVGLSLGKNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 54 - protein, 336 aa, Artificial Sequence
  HS3 excluding signal sequence, flexible N-terminal and flexible loop and comprising a Bip leader sequence
MKLCILLAVVAFVGLSLGSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQ

SEQ ID NO: 55 - protein, 362 aa, Artificial Sequence
  HS3 excluding signal sequence, flexible N-terminal and flexible loop and comprising a Bip leader sequence and a Hexa-histidine C-terminal tag
MKLCILLAVVAFVGLSLGKNVNFLQYHFKELSNYNIANSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQHHHHHH

SEQ ID NO: 56 - protein, 342 aa, Artificial Sequence
  HS3 excluding signal sequence, flexible N-terminal and flexible loop and comprising a Bip leader sequence and a Hexa-histidine C-terminal tag
MKLCILLAVVAFVGLSLGSIDILQEKEGHLDFVIIPHYTFLEYYKHLSYIAIYHKSSTYGKYIAVDAFIKKINEAYDKVKSKCNDIKNDLINTIKKLEHPYDINNKNRAFKKMMDEYNTKKKKLIKCIKNHENDFNKICMDMKNYGTNLFEQLSCYNNNFCNTNGIRYHYDEYIHKLINSVKSKNLNKDLNDMTNILQQSEDLLTNLKKKMGSYIYIDTIKFIHKEMKHIFNRIEYHTKIINDKTKIIQDKIKLDIWRTFQKDELLKRILDMAKEYALFRTSDDLRQMLYNTFYSKEKHLNNIFHHLIYVLQMKFNDVPIPMEYFQTYKKNKPLTQHHHHHH

(19) United States
(12) Patent Application Publication
(10) Pub. No.: US 2019/0374629 A1
(43) Pub. Date: Dec. 12, 2019

(54) THERMOSTABLE VARIANTS OF P. FALCIPARUM PFRH5 WHICH CAN BE PRODUCED IN BACTERIAL CELLS

(71) Applicants: OXFORD UNIVERSITY INNOVATION LIMITED, Oxford, Oxfordshire (GB); YEDA RESEARCH AND DEVELOPMENT CO. LTD., Rehovot (IL)

(72) Inventors: Simon J. Draper, Oxford (GB); Ivan Campeotto, Oxford (GB); Matthew K. Higgins, Oxford (GB); Sarel Fleishman, Rehovot (IL); Adi Goldenzweig, Rehovot (IL)

(21) Appl. No.: 16/331,863

(22) PCT Filed: Sep. 7, 2017
(86) PCT No.: PCT/GB2017/052608

§ 371 Date: Mar. 8, 2019

(30) Foreign Application Priority Data
Sep. 8, 2016 (GB) 1615298.5

(51) Int. Cl. A61K 39/015; C07K 14/445; A61P 33/06

(52) U.S. Cl. CPC A61K 39/015; A61P 33/06; C07K 14/445

(57) ABSTRACT

There are provided antigens, vectors encoding the antigens, and antibodies and other binding compounds to the antigens and uses thereof in the prevention or treatment of malaria. In particular, compositions are provided comprising Reticulocyte-binding protein Homologue 5 (PfRH5) antigens. In particular, the invention provides modified PfRH5 antigens rationally designed to produce improved stability and expression profiles whilst maintaining immunogenicity.

### FIELD OF THE INVENTION

The present invention relates to antigens, antibodies and vaccines for treatment or prevention of malaria.

### BACKGROUND OF THE INVENTION

Malaria places the gravest public-health burden of all parasitic diseases, leading to ˜215 million human clinical cases and ˜440,000 deaths annually, with the majority of deaths in children. The infection of red blood cells (RBCs) by the blood-stage form of the Plasmodium parasite is responsible for the clinical manifestations of malaria. Examples of Plasmodium parasite include the species P. falciparum, P. vivax, P. ovale and P. malariae. The most virulent parasite species, P. falciparum, is endemic in large parts of sub-Saharan Africa and Latin America. It causes the majority of malaria deaths. It can infect RBCs of all ages and is not limited to immature RBCs. P. falciparum, is therefore of particular interest and is a major target for vaccine development, as it would be highly desirable to develop a vaccine.

There are currently no licensed malaria vaccines on the market. The most advanced current vaccine candidates are based on the RTS,S protein, which acts by blocking infection of P. falciparum in the liver. The leading RTS,S vaccine candidate has achieved only partial efficacy (˜30-50% in phase II/III clinical trials). There is therefore an urgent need for a vaccine which can emulate natural immunity by protecting against the disease-causing blood-stage Plasmodium parasite.

Previous studies have investigated the potential for antigens to induce antibodies which are effective against blood-stage malaria parasites in vitro, using the standard growth inhibitory activity (GIA) assay. One such antigen is apical membrane antigen 1 (PfAMA1).

GIA assay investigations into other protein families involved in blood-stage Plasmodium parasite invasion of RBCs have found them to be ineffective or less effective than PfAMA1.

PfAMA1 has therefore been a major focus of research on countering blood-stage malarial parasites, with ongoing clinical trials. However, antibodies against PfAMA1 appear only to be effective at an extremely high concentration. In addition, PfAMA1 induces strain-specific antibodies which are not effective against genetically diverse strains of the Plasmodium parasite (A. L. Goodman, S. J. Draper, Ann. Trop. Med. Parasitol. 104, 189 (2010)). In addition, vaccine development has been hampered by the requirement for potentially reactogenic chemical adjuvants in addition to the antigen to induce sufficient antibody responses in human subjects.

Research has also been ongoing to identify other candidate malarial antigens for vaccines. In particular, the present inventors have previously identified Reticulocyte-binding protein Homologue 5 (PfRH5) as a potential antigen candidate for malarial vaccines (WO 2012/114125).

The Reticulocyte binding Homologue (PfRH) family comprises six members (PfRH1, PfRH2a, PfRH2b, PfRH3, PfRH4 and PfRH5), each of which is involved in the binding of the Plasmodium parasite to RBCs, with the possible exception of PfRH3 which may be a non-expressed pseudogene. The PfRH family has been identified as adhesins on the surface of the merozoite form of the Plasmodium parasite, which bind to receptors on the surface of the erythrocyte and hence permit invasion of RBCs by the parasite in its blood-stage. The PfRH5 antigen has an approximate molecular weight of 63 KDa. In vitro cleaved fragments of approximately 45 KDa and 28 KDa have been reported.

The present inventors have previously demonstrated that PfRH5 induces antibodies which are highly effective in the GIA assay against the blood-stage Plasmodium parasite and which neutralise parasites more effectively than PfAMA1 and remain effective at lower concentrations of immunoglobulin. In addition, PfRH5 induces antibodies which are effective against genetically diverse strains of the Plasmodium parasite. Therefore, PfRH5 is a promising candidate antigen for a malarial vaccine.

Earlier work by the present inventors has improved upon the full-length PfRH5 as a vaccine candidate by the development of rationally designed PfRH5 fragments, which contain regions or amino acid residues from within PfRH5 that give rise to protective antibodies, whilst excluding other regions of the full-length PfRH5 sequence which may be associated with unwanted side effects.

However, despite this promise, PfRH5 suffers from two significant shortcomings as a subunit vaccine candidate. First, the protein has limited stability at high temperatures, and second, despite extensive protein engineering, correctly folded, soluble, functional PfRH5 has not been produced in microbial expression hosts. Instead, production has relied on more expensive eukaryotic expression systems, such as transiently transfected HEK293 cells or stable insect cell lines. This is problematic, given that the most likely use for PfRH5-based vaccines would involve population-wide inoculation in hot and underdeveloped regions, where a cold-chain for transporting vaccine formulations is impracticable.

Therefore, there is an ongoing need for the development of rationally designed antigens with improved properties. In particular, there is a need for improved antigens that will induce antibodies that are effective at low concentrations of immunoglobulin, for improved antigens that will induce antibodies that are effective against genetically diverse strains of the Plasmodium parasite, and for improved antigens that are effective without requiring potentially reactogenic chemical adjuvants. Further, there is a need to provide antigens that can be produced more inexpensively. In particular, there is a need for a stabilized variant that can be cheaply produced as soluble protein in microbial cells, and will retain efficacy when stored at elevated temperatures.

The present invention addresses one or more of the above needs by providing antigens, vectors encoding the antigens, and antibodies (and antibody-like molecules including aptamers and peptides) raised against the antigen, together with the use thereof (either alone or in combination) in the prevention or treatment of malaria. Antibodies and antibody-like molecules raised against the antigen may bind (e.g. specifically bind) to the antigen.

### SUMMARY OF THE INVENTION

The present inventors have previously described a stability-design algorithm PROSS (see, PCT/IL2016/050812 and Goldenzweig, A. et al. [Mol Cell, 2016, 63(2), pp. 337-46], which are incorporated herein by reference), and demonstrated its effectiveness in designing modified forms of human enzymes with improved thermal stability. Until now, PfRH5 has presented an unusual challenge for sequence analysis because of the high level of sequence identity between the PfRH5 proteins of different P. falciparum strains. The inventors have for the first time now shown that a computational design algorithm can be used to generate modified PfRH5 antigens, despite the high level of sequence identity between different PfRH5 proteins. The inventors have also demonstrated that said modified PfRH5 antigens have improved expression profiles and thermal stability without compromising immunological efficacy.

Accordingly, the present invention provides a vaccine composition comprising a modified Reticulocyte-binding protein Homologue 5 (PfRH5) antigen, wherein said modified PfRH5 antigen comprises an amino acid substitution at five or more of amino acid positions 183, 233, 381, 392, 398, 464, 467, 57, 164, 171, 178, 188, 191, 192, 195, 221, 230, 231, 234, 236, 300, 304, 305, 308, 309, 311, 312, 314, 315, 316, 330, 336, 354, 365, 368, 369, 370, 384, 390, 391, 394, 395, 396, 401, 406, 414, 422, 424, 428, 435, 442, 444, 445, 455, 458, 463, 468, 470, 474, 479, 481, 485, 495, 505 and/or 511, or any combination thereof, relative to the corresponding unmodified PfRH5 antigen.

Typically, said modified PfRH5 antigen comprises an amino acid substitution at each of positions 183, 233, 381, 392, 398, 464 and/or 467 relative to the corresponding unmodified PfRH5 antigen. Said modified PfRH5 antigen may further comprise one or more amino acid substitution at position 157, 191, 192, 236, 304, 308, 312, 314, 316, 330, 369, 370, 384, 395, 414, 444, 445, 458, 463, 470, 474, 495, 505 and/or 511, or any combination thereof, relative to the corresponding unmodified PfRH5 antigen.

In a preferred embodiment, said modified PfRH5 antigen comprises amino acid substitutions at: (a) positions 157, 183, 233, 304, 312, 314, 316, 330, 370, 381, 384, 392, 395, 398, 458, 464, 467 and 505 relative to the corresponding unmodified PfRH5 antigen; (b) positions 183, 191, 192, 233, 369, 381, 392, 398, 445, 463, 464, 467, 470, 474 and 511 relative to the corresponding unmodified PfRH5 antigen; or (c) positions 183, 191, 192, 233, 236, 308, 314, 369, 370, 381, 384, 392, 395, 398, 414, 444, 445, 463, 464, 467, 470, 474, 495, 505 and 511 relative to the corresponding unmodified PfRH5 antigen.

Said one or more amino acid substitution may be a conservative amino acid substitution or a non-conservative amino acid substitution. Said one or more amino acid substitution may be a substitution by a leucine, glutamic acid, phenylalanine, asparagine, alanine, lysine, isoleucine, aspartic acid, arginine, proline, or histidine. In some embodiments, the amino acid at position: (i) 157 is substituted by a leucine; (ii) 183 is substituted by a glutamic acid; (iii) 191 is substituted by an isoleucine; (iv) 192 is substituted by an alanine; (v) 233 is substituted by a lysine or asparagine; (vi) 236 is substituted by a histidine; (vii) 304 is substituted by a phenylalanine; (viii) 308 is substituted by a lysine; (ix) 312 is substituted by an asparagine; (x) 314 is substituted by a phenylalanine; (xi) 316 is substituted by an asparagine; (xii) 330 is substituted by an asparagine; (xiii) 369 is substituted by an asparagine; (xiv) 370 is substituted by an alanine or lysine; (xv) 381 is substituted by an asparagine; (xvi) 384 is substituted by a lysine; (xvii) 392 is substituted by a lysine or aspartic acid; (xviii) 395 is substituted by an asparagine or arginine; (xix) 398 is substituted by a glutamic acid or lysine; (xx) 414 is substituted by a leucine; (xxi) 444 is substituted by a glutamic acid; (xxii) 445 is substituted by an aspartic acid; (xxiii) 458 is substituted by a lysine; (xxiv) 463 is substituted by an alanine; (xxv) 464 is substituted by a lysine; (xxvi) 467 is substituted by an alanine; (xxvii) 470 is substituted by an arginine; (xxviii) 474 is substituted by an aspartic acid; (xxix) 495 is substituted by an asparagine; (xxx) 505 is substituted by a leucine; and/or (xxxi) 511 is substituted by a proline; or any combination thereof.

In a preferred embodiment, the modified PfRH5 antigen comprises the following amino acid substitutions: (a) I157L, D183E, A233K, M304F, K312N, L314F, K316N, M330N, S370A, S381N, T384K, L392K, T395N, N398E, R458K, N464K, S467A and F505L, (b) D183E, N191I, S192A, A233N, L369N, S381N, T392D, N398K, N445D, S463A, N464K, S467A, I470R, H474D and K511P; or (c) D183E, N191I, S192A, A233N, K236H, N308K, L314F, L369N, S370K, S381N, T384K, T392D, T395R, N398K, H414L, L444E, N445D, S463A, N464K, S467A, I470R, H474D, H495N, F505L and K511P.

Typically one or more of amino acid positions 147, 149, 193, 194, 196, 197, 198, 200, 201, 202, 203, 204, 205, 206, 207, 209, 212, 213, 216, 222, 225, 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and/or 496, or any combination thereof, is unchanged in the modified PfRH5 antigen relative to the corresponding unmodified PfRH5 antigen. Preferably all of amino acid positions 147, 149, 193, 194, 196, 197, 198, 200, 201, 202, 203, 204, 205, 206, 207, 209, 212, 213, 216, 222, 225 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and 496 are unchanged in the modified PfRH5 antigen relative to the corresponding unmodified PfRH5 antigen.

The unmodified PfRH5 antigen may comprise at least 90% sequence identity to SEQ ID NO: 1 or 2.

The unmodified PfRH5 antigen may comprise a basigin-binding fragment of PfRH5 comprising: (a) amino acid residues 140 to 526 of SEQ ID NO: 1 or 2, or a fragment of an amino acid sequence having at least 90% sequence identity to amino acid residues 140 to 526 of SEQ ID NO: 1 or 2; or (b) amino acid residues 160 to 526 of SEQ ID NO: 1 or 2, or a fragment of an amino acid sequence having at least 90% sequence identity to amino acid residues 160 to 526 of SEQ ID NO: 1 or 2. In such embodiments, said fragment of PfRH5 may comprise the amino acid sequence of any one of SEQ ID NOs: 3 to 6, or an amino acid sequence having at least 90% sequence identity to one of SEQ ID NOs: 3 to 6.

The unmodified PfRH5 antigen may comprise a discontinuous fragment of PfRH5, wherein optionally said discontinuous fragment of PfRH5 lacks the flexible loop region corresponding to amino acid residues 248 to 296 of SEQ ID NO: 1 or 2. Preferably said discontinuous fragment of PfRH5 has at least 90% sequence identity to any one of SEQ ID NO: 7 to 14, preferably SEQ ID NO: 7 to 10.

In preferred embodiments, the modified PfRH5 antigen comprises the amino acid sequence of any one of SEQ ID NOs: 15 to 56, more preferably any one of SEQ ID NOs: 21 to 28, 35 to 42 or 49 to 56.

Typically a vaccine composition of the invention induces antibodies that have a growth inhibitory activity (GIA) of at least 50% at a concentration of 10 mg/ml.

The composition may further comprise one or more antigens selected from PfAMA1, PfEBA175, PfRH1, PfRH2a, PfRH2b, PfRH4, PfCyRPA, PfRIPR, PfP113 and/or PfAARP, or a fragment thereof.

The modified PfRH5 antigen in a composition of the invention may be in the form of a recombinant protein, a protein particle, a virus-like particle, a fusion protein, or a combination thereof.

The composition of the invention may comprise a fusion of the modified PfRH5 antigen and one or more antigens selected from PfAMA1, PfEBA175, PfRH1, PfRH2a, PfRH2b, PfRH4, PfCyRPA, PfRIPR, PfP113 and/or PfAARP, or a fragment thereof.

The invention further provides a viral vector, RNA vaccine or DNA plasmid that expresses a modified PfRH5 antigen of the invention. Said viral vector, RNA vaccine or DNA plasmid may express said modified PfRH5 antigen, further comprising a signal peptide. Typically, the signal peptide directs secretion from human cells and/or is a mammalian signal peptide from tissue plasminogen activator.

The viral vector, RNA vaccine or DNA plasmid of the invention may further express one or more antigens selected from PfAMA1, PfEBA175, PfRH1, PfRH2a, PfRH2b, PfRH4, PfCyRPA, PfRIPR, PfP113 and/or PfAARP, or a fragment thereof. Said viral vector, RNA vaccine or DNA plasmid may express said modified PfRH5 antigen and one or more antigens selected from PfAMA1, PfEBA175, PfRH1, PfRH2a, PfRH2b, PfRH4, PfCyRPA, PfRIPR, PfP113 and/or PfAARP, or a fragment thereof, as a fusion protein.

The invention further provides a viral vector, RNA vaccine or DNA plasmid which expresses a modified PfRH5 antigen of the invention, in combination with a viral vector, RNA vaccine or DNA plasmid that expresses one or more antigens selected from PfAMA1, PfEBA175, PfRH1, PfRH2a, PfRH2b, PfRH4, PfCyRPA, PfRIPR, PfP113 and/or PfAARP, or a fragment thereof.

The viral vector of the invention may be a human or simian adenovirus, or a pox virus. Said viral vector may be an AdHu5, ChAd63, ChAdOX1, ChAdOX2 or modified vaccinia Ankara (MVA) vector.

Typically the RNA vaccine or DNA plasmid of the invention is capable of expression in an immunised mammalian cell. The DNA plasmid of the invention may be capable of expression in a heterologous protein expression system.

The invention further provides an antibody, or binding fragment thereof, that specifically binds to a modified PfRH5 antigen of the invention. Said antibody, or binding fragment thereof, may be a monoclonal or polyclonal antibody. The antibody, or binding fragment thereof, may be a Fab, F(ab′)2, Fv, scFv, Fd or dAb.

The invention also provides an oligonucleotide aptamer that specifically binds to a modified PfRH5 antigen of the invention.

The invention also provides a vaccine composition comprising the viral vector, and/or RNA vaccine and/or DNA plasmid of the invention.

The invention further provides a vaccine composition, viral vector, RNA vaccine, DNA plasmid, antibody and/or aptamer of the invention for use in the treatment and/or prevention of malaria.

The invention further provides the use of a vaccine composition, viral vector, RNA vaccine, DNA plasmid, antibody and/or aptamer of the invention in the manufacture of a medicament for the prevention and/or treatment of malaria.

According to the present invention, the treatment and/or prevention of malaria may comprise priming a subject with a human or simian adenovirus, for example AdHu5, ChAd63, ChAdOX1 or ChAdOX2. The treatment and/or prevention of malaria may further comprise boosting a subject with a pox virus, for example MVA.

The invention further provides a vaccine composition, viral vector, RNA vaccine and/or DNA plasmid of the invention for use in immunising a subject, wherein the modified PfRH5 antigen results in antibodies with a growth inhibitory activity (GIA) of at least 50% against the blood-stage Plasmodium parasite. In some embodiments, the modified PfRH5 antigen results in antibodies with a growth inhibitory activity (GIA) of at least 50% against a plurality of genetic strains of the blood-stage Plasmodium parasite. The Plasmodium parasite may be Plasmodium falciparum.

The invention further provides a host cell containing a recombinant expression vector which encodes for a modified PfRH5 antigen of the invention, wherein optionally the host cell is an insect cell, preferably a Drosophila melanogaster cell, or an Escherichia coli cell.

### DESCRIPTION OF FIGURES

FIG. 1: Design of an E. coli expressible modified PfRH5 antigen. A. 80% of mutations in all three designed modified antigens are located in the C-terminal half, where most of the aligned sequences contribute information on sequence diversity. The PfRh5 sequence is schematically shown from N- to C-terminus, and each position is coloured according to the number of unique sequences contributing to the strict and permissive alignments, ranging from one to eight and from one to fourteen respectively. The locations of the mutations in each designed variant relative to wild-type (unmodified) PfRH5ΔNL are indicated by triangles. B. Expression levels of PfRH5ΔNL (RH5), PfRH5ΔNL_{HS1} (HS1), PfRH5ΔNL_{HS2} (HS2) and PfRH5ΔNL_{HS3} (HS3) from E. coli. ‘Total’ is whole cells, ‘soluble’ is material after cell lysis and clarification and ‘final’ is after IMAC and size exclusion chromatography. C. Expression levels of PfRH5ΔNL (RH5) and PfRH5ΔNL_{HS1} (HS1) secreted in the cell supernatants from a stable S2 cell line. D. Surface plasmon resonance (SPR) analysis of the binding of PfRH5ΔNL_{HS1} to basigin, with two fold dilutions of PfRH5ΔNL_{HS1} from a top concentration of 8 μM. E. In vitro GIA of purified IgG against 3D7 clone parasites from mice immunized with either PfRH5ΔNL or PfRH5ΔNL_{HS1}. The anti-PfRH5 antibody response was measured for each sample of purified IgG and is plotted against the measured level of GIA. The dashed line indicates 50% GIA, and each GIA datapoint represents the mean of each sample tested in triplicate. F. The structure of PfRH5ΔNL_{HS1}: 9AD4 (light grey) overlaid on the structure of PfRH5ΔNL:9AD4 (dark grey).

FIG. 2: Surface plasmon resonance (SPR) characterization of PfRH5ΔNL and PfRH5ΔNL_{HS1}. Surface plasmon resonance analysis of the binding of PfRH5ΔNL and PfRH5ΔNL_{HS1} to basigin, with two fold dilutions of PfRH5ΔNL_{HS1} from a top concentration of 8 μM. The same analysis was performed after lyophilizing both PfRH5ΔNL and PfRH5ΔNL_{HS1} before measurement.

FIG. 3: Structural characterization of PfRH5ΔNL and PfRH5ΔNL_{HS1}. The structures of A. PfRH5ΔNL and B. PfRH5ΔNL_{HS1}, bound to the Fab fragment of the monoclonal antibody 9AD4. C. The structure of PfRH5ΔNL_{HS1} in grey, with residues that are different to those in PfRH5ΔNL show as sticks. A composite omit map, contoured at 1.0 r.m.s.d level, shows the electron density for the residues that differ between PfRH5ΔNL and PfRH5ΔNL_{HS1}.

FIG. 4: Increased thermal stability of variant PfRH5ΔNL_{HS1}. A. Determination of the effect of temperature on the ellipticity of PfRH5ΔNL (dark grey) and PfRH5ΔNL_{HS1} (light grey) at a wavelength of 220 nm as measured by circular dichroism. B, C. Determination of the effect of temperature on the binding of PfRH5ΔNL and PfRH5ΔNL_{HS1} to basigin, measured by surface plasmon resonance (SPR). Protein, at 16 μM, was incubated for 60 minutes at the specified temperature before analysis at 8 μM.

FIG. 5: Structural insights into thermal stability of PfRH5ΔNL_{HS1}. The structural underpinnings of stabilization in PfRH5ΔNL_{HS1}. Wild-type PfRH5 is shown in stick representation and the 18 mutated positions, throughout PfRH5ΔNL_{HS1}, are indicated by spheres. Thumbnails highlight stabilizing effects of selected mutations.

FIG. 6: Biophysical characterization of PfRH5ΔNL_{HS1} confirms its purity and homogeneity A. Coomassie stained gel of purified PfRH5ΔNL_{HS1}. B. Analysis by size exclusion column multi-angle light scattering (SEC-MALS) of purified PfRH5ΔNL_{HS1}.

### DETAILED DESCRIPTION OF THE INVENTION

### Modified PfRH5 Antigens

The present invention provides modified PfRH5 antigens. By modified, it is meant that an antigen of the invention will differ in amino acid sequence from the corresponding unmodified PfRH5 antigen, typically in a way that improves the stability and/or expression profile of the modified PfRH5 antigen as described herein. In particular, using the PROSS methodology, the present inventors have for the first time identified key amino acid positions within the wildtype PfRH5 amino acid sequence (also known as the native sequence and herein described as the unmodified PfRH5 sequence) which can be modified according to the present invention to produce modified PfRH5 antigens. As described and exemplified herein, the modified PfRH5 antigens of the invention are typically more stable than the corresponding unmodified PfRH5 antigens, and also have improved expression profiles.

An amino acid modification according to the invention may be a substitution, deletion, addition or other modification, including post-translational modification, unless the relevant disclosure explicitly says otherwise. Preferably said modifications are amino acid substitutions. Said modifications have been devised using the methodology described herein to improve the stability and/or expression profile of the modified PfRH5 antigen.

A modified PfRH5 antigen of the invention comprises a modification, preferably an amino acid substitution, at one or more of the amino acid positions identified by the use of a computational method, known as PROSS relative to the corresponding unmodified PfRH5 antigen. These amino acid positions are listed in Table 1 herein, together with preferred amino acid substitutions. Thus, one or more of amino acid positions 157, 164, 171, 178, 183, 188, 191, 192, 195, 221, 230, 231, 233, 234, 236, 300, 304, 305, 308, 309, 311, 312, 314, 315, 316, 330, 336, 354, 365, 368, 369, 370, 381, 384, 390, 391, 392, 394, 395, 396, 398, 401, 406, 414, 422, 424, 428, 435, 442, 444, 445, 455, 458, 463, 464, 467, 468, 470, 474, 479, 481, 485, 495, 505 and/or 511 may be modified relative to the corresponding amino acid positions in the corresponding unmodified PfRH5 antigen. Typically these amino acid positions are defined relative to a full-length unmodified PfRH5 antigen as described herein, such as SEQ ID NO: 1 or 2. Accordingly, a reference to any given position may be interpreted as a reference to said position in a full-length unmodified PfRH5 antigen, or a position corresponding thereto.

A modified PfRH5 antigen of the invention may comprise a modification, preferably an amino acid substitution at: at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 30, at least 40, at least 50, at least 60 or more of the listed amino acid positions, or any combination thereof. Typically, a modified PfRH5 antigen of the invention may comprise a modification, preferably an amino acid substitution at: at least 15, at least 16, at least 17, at least 18, at least 19, at least 20, at least 21, at least 22, at least 23, at least 24, at least 25, at least 30, at least 40, at least 50, at least 60 or more of the listed amino acid positions, or any combination thereof.

Typically a modified PfRH5 antigen of the invention may comprise an amino acid modification, preferably a substitution at one or more of positions 183, 233, 381, 392, 398, 464 and/or 467 relative to the corresponding unmodified PfRH5 antigen. Any combination of any two, any three, any four, any five, any six, or all seven of these positions may be modified (preferably substituted) according to the present invention. In preferred embodiments, a modified PfRH5 antigen of the invention comprises an amino acid modification, preferably a substitution, at each of positions 183, 233, 381, 392, 398, 464 and/or 467 relative to the corresponding unmodified PfRH5 antigen.

In some embodiments, the modified PfRH5 antigen of the invention further comprises one or more amino acid modification at position 157, 191, 192, 236, 304, 308, 312, 314, 316, 330, 369, 370, 384, 395, 414, 444, 445, 458, 463, 470, 474, 495, 505 and/or 511, or any combination thereof, relative to the corresponding unmodified PfRH5 antigen. Preferably said modifications are amino acid substitutions. As a non-limiting example, the modified PfRH5 antigen of the invention may comprise a modification (preferably an amino acid substitution) at least two, at least three, at least four, at least five, at least six, at least seven, at least eight, at least nine, at least ten or more of these positions relative to the corresponding unmodified PfRH5 antigen.

In some embodiments, a modified PfRH5 antigen of the invention comprises amino acid modification at each of positions 183, 191, 192, 233, 369, 381, 392, 398, 445, 463, 464, 467, 470, 474 and 511 relative to the corresponding unmodified PfRH5 antigen. Alternatively, a modified PfRH5 antigen of the invention comprises amino acid substitutions at each of positions 183, 191, 192, 233, 236, 308, 314, 369, 370, 381, 384, 392, 395, 398, 414, 444, 445, 463, 464, 467, 470, 474, 495, 505 and 511 relative to the corresponding unmodified PfRH5 antigen. In each case, preferably said modifications are amino acid substitutions.

In a preferred embodiment, a modified PfRH5 antigen of the invention comprises amino acid modifications (preferably amino acid substitutions) at each of positions 157, 183, 233, 304, 312, 314, 316, 330, 370, 381, 384, 392, 395, 398, 458, 464, 467 and 505 relative to the corresponding unmodified PfRH5 antigen. In a particularly preferred embodiment, a modified PfRH5 antigen of the invention comprises amino acid substitutions at each of positions 157, 183, 233, 304, 312, 314, 316, 330, 370, 381, 384, 392, 395, 398, 458, 464, 467 and 505 relative to the corresponding unmodified PfRH5 antigen.

In addition to the preferred positions (157, 183, 191, 192, 233, 236, 304, 308, 312, 314, 316, 330, 369, 370, 381, 384, 392, 395, 398, 414, 444, 445, 458, 463, 464, 467, 470, 474, 495, 505 and/or 511, or any combination thereof) identified above, a modified PfRH5 antigen of the invention may comprise a modification (preferably an amino acid substitution) at one or more further amino acid position relative to the corresponding unmodified PfRH5 antigen. Typically the one or more further amino acid position that may be modified is identified using the PROSS method. Amino acid positions that may be modified according to the present invention are listed in Table 1 below. Thus, a modified PfRH5 antigen of the invention may additionally comprise a modification (preferably an amino acid substitution) at one or more of amino acid positions 164, 171, 178, 188, 195, 221, 230, 231, 234, 300, 305, 309, 311, 315, 330, 336, 354, 365, 368, 390, 391, 394, 396, 401, 406, 422, 424, 428, 435, 442, 455, 458, 468, 479, 481 and/or 485, or any combination thereof. A modified PfRH5 antigen of the invention may additionally comprise a modification, preferably an amino acid substitution at: at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 30, at least 40, at least 50, at least 60 or more of said amino acid positions, or any combination thereof.

The amino acid positions to be modified according to the present invention are preferably modified by substitution. In other words, the amino acid at a specified position within the wildtype (unmodified) PfRH5 sequence is substituted by a naturally occurring or non-naturally occurring amino acid that is different to the amino acid present at that position in the unmodified PfRH5 sequence. Alternatively, the amino acid at a specified position within the wildtype (unmodified) PfRH5 sequence may be modified post-translationally. Post-translational modifications include glycosylations, acetylations, acylations, de-aminations, phosphorylisations, isoprenylisations, glycosyl phosphatidyl inositolisations and further modifications known to a person skilled in the art.

The modification of one or more amino acid position as described herein may be performed, for example, by specific mutagenesis, or any other method known in the art.

In embodiments in which one or more amino acid position is substituted relative to the corresponding unmodified PfRH5 antigen, the substitution may be a conservative substitution or a non-conservative substitution. A conservative substitution is defined as substitution by an amino acid pertaining to the same physiochemical group to the amino acid present in the unmodified PfRH5 antigen. A non-conservative amino acid substitution is defined as substitution by an amino acid pertaining to a different physiochemical group to the amino acid present in the unmodified PfRH5 antigen. In more detail, amino acids are, in principle, divided into different physiochemical groups. Aspartate and glutamate belong to the negatively-charged amino acids. Histidine, arginine and lysine belong to the positively-charged amino acids. Asparagine, glutamine, serine, threonine, cysteine and tyrosine belong to the polar amino acids. Glycine, alanine, valine, leucine, isoleucine, methionine, proline, phenylalanine and tryptophan belong to the non-polar amino acids. Aromatic side groups are to be found among the amino acids histidine, phenylalanine, tyrosine and tryptophan. Thus, as a non-limiting example, a conservative substation may involve the substitution of a non-polar amino acid by another non-polar amino acid, such as substituting leucine with isoleucine. As another non-limiting example, a non-conservative substitution may involve the substation of a non-polar amino acid (e.g. leucine) with a negatively-charged amino acid (e.g. aspartate), a positively-charged amino acid (e.g. arginine), or a polar amino acid (e.g. asparagine).

Without wishing to be bound by theory, one or more amino acid substitution present in a modified PfRH5 antigen of the invention may improve surface polarity and/or eliminate a homogenously positively charged region on the protein surface (such regions can be associated with protein aggregation and poor stability). Alternatively or in addition, one or more amino acid substitution present in a modified PfRH5 antigen of the invention may increase the helix-forming propensity of the modified PfRH5 antigen relative to the corresponding unmodified PfRH5 antigen, which may also serve to increase stability. Lastly, and again alternatively or in addition, one or more amino acid substitution present in a modified PfRH5 antigen of the invention may improve packing of the hydrophobic core of the modified PfRH5 antigen relative to the corresponding unmodified PfRH5 antigen, which may also serve to increase stability.

At least one amino acid substitution as described herein may be a conservative amino acid substitution, such as substituting one negatively charged amino acid for another. Other conservative amino acid substitutions encompassed by the present invention include substituting a hydrophobic amino acid by another hydrophobic amino acid, substituting a positively-charged amino acid by another positively-charged amino acid, or substituting a polar amino acid by another polar amino acid.

At least one amino acid substitution as described herein may be a non-conservative amino acid substitution, such as substituting a polar amino acid with a hydrophobic amino acid. Other non-conservative amino acid substitutions encompassed by the present invention include, but are not limited to, substituting a hydrophobic amino acid by a positively-charged amino acid, substituting a hydrophobic amino acid by a negatively-charged amino acid, substituting a hydrophobic amino acid by a polar amino acid, substituting a polar amino acid by a positively- or negatively-charged amino acid, or by a hydrophobic amino acid, substituting a positively- or negatively-charged amino acid by a polar or hydrophobic amino acid, or by substituting a positively-charged amino acid by a negatively-charged amino acid, or vice versa.

In some embodiments, at least one amino acid substitution at one of the above-recited positions in a modified PfRH5 antigen of the invention is a substitution by a leucine, glutamic acid, phenylalanine, asparagine, alanine, lysine, isoleucine aspartic acid, arginine, proline, or histidine.

Typically, a modified PfRH5 antigen of the invention comprises one or more amino acid substitution relative to the corresponding unmodified PfRH5 antigen. These amino acid substitutions are listed in Table 1 herein. Thus, a modified PfRH5 antigen of the invention may comprise one or more of the following amino acid substitutions: I157L, L164F, L171V, H178Y, D183E or D183Y, L188T or L188V, N191I, S192A, H195Y, K221I, D230E, L231F, A233N, A233T or A233K, T234L, K236H, F300Y, M304I or M304F, D305N, N308K, T309K, K311I, K312N, L314F or L314Y, I315H or I315M, K316N or K316Q, M330N, G336S, N354P, H365R, I368M, L369N, S370E, S370K, S370N, or S370A, S381D, S381K or S381N, T384K, S390A, E391I, L392D or L392K, L394I or L394V, T395N or T395R, N396K, N398E, N398K or N398R, M401I, Y406V, H414I or H414L, N422E, I424F or I424M, T428I, T435I or T435Y, I442F, I442K or I442Y, L444D, L444E, L444N or L444Q, N445D or N445V, L455F, R458K, S463A or S463V, N464K, S467A, L468I, I470K or I470R, H474D, L479F, N481K, S485H, S485L or S485T, H495N, F505L and/or K511P relative to the corresponding amino acid positions in the corresponding unmodified PfRH5 antigen. Typically these amino acid positions are defined relative to a full-length unmodified PfRH5 antigen as described herein, such as SEQ ID NO: 1 or 2. Accordingly, a reference to any given position may be interpreted as a reference to said position in a full-length unmodified PfRH5 antigen, or a position corresponding thereto. A modified PfRH15 antigen of the invention may comprise at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 30, at least 40, at least 50, at least 60 or more of the listed amino acid substitutions, or any combination thereof.

In a preferred embodiment, a modified PfRH5 antigen of the invention comprises at least one, at least two, at least three at least four, at least five, at least six, at least seven, at least eight, at least nine, at least ten, at least 11, at least 12, at least 13, at least 14, at least 15, at least 16, at least 17, at least 18, at least 19, at least 20, or more of the following substitutions: position 157 is substituted by a leucine, position 183 is substituted by a glutamic acid, position 191 is substituted by an isoleucine, position 192 is substituted by an alanine, position 233 is substituted by a lysine or asparagine, position 236 is substituted by a histidine, position 304 is substituted by a phenylalanine, position 308 is substituted by a lysine, position 312 is substituted by an asparagine, position 314 is substituted by a phenylalanine, position 316 is substituted by an asparagine, position 330 is substituted by an asparagine, position 369 is substituted by an asparagine, position 370 is substituted by an alanine or lysine, position 381 is substituted by an asparagine, position 384 is substituted by a lysine, position 392 is substituted by a lysine or aspartic acid, position 395 is substituted by an asparagine or arginine, position 398 is substituted by a glutamic acid or lysine, position 414 is substituted by a leucine, position 444 is substituted by a glutamic acid, position 445 is substituted by an aspartic acid, position 458 is substituted by a lysine, position 463 is substituted by an alanine, position 464 is substituted by a lysine, position 467 is substituted by an alanine, position 470 is substituted by an arginine, position 474 is substituted by an aspartic acid, position 495 is substituted by an asparagine, position 505 is substituted by a leucine, and/or position 511 is substituted by a proline, or any combination thereof. These particular amino acid substitutions are preferred in embodiments in which a modified PfRH5 antigen of the invention comprises amino acid substitutions at each of: (i) positions 183, 191, 192, 233, 369, 381, 392, 398, 445, 463, 464, 467, 470, 474 and 511 relative to the corresponding unmodified PfRH5 antigen; (ii) positions 183, 191, 192, 233, 236, 308, 314, 369, 370, 381, 384, 392, 395, 398, 414, 444, 445, 463, 464, 467, 470, 474, 495, 505 and 511 relative to the corresponding unmodified PfRH5 antigen; or (iii) positions 157, 183, 233, 304, 312, 314, 316, 330, 370, 381, 384, 392, 395, 398, 458, 464, 467 and 505 relative to the corresponding unmodified PfRH5 antigen.

In preferred embodiments, a modified PfRH5 antigen of the invention comprises the amino acid substitutions I157L, D183E, A233K, M304F, K312N, L314F, K316N, M330N, S370A, S381N, T384K, L392K, T395N, N398E, R458K, N464K, S467A and F505L. In other preferred embodiments, a modified PfRH5 antigen of the invention comprises the amino acid substitutions D183E, N191I, S192A, A233N, L369N, S381N, T392D, N398K, N445D, S463A, N464K, S467A, I470R, H474D and K511P. In still other preferred embodiments, a modified PfRH5 antigen of the invention comprises the amino acid substitutions D183E, N191I, S192A, A233N, K236H, N308K, L314F, L369N, S370K, S381N, T384K, T392D, T395R, N398K, H414L, L444E, N445D, S463A, N464K, S467A, I470R, H474D, H495N, F505L and K511P.

In addition to the preferred amino acid substitutions listed above, a modified PfRH5 antigen of the invention may comprises one or more additional amino acid substitution as described herein relative to the corresponding unmodified PfRH5 antigen (see Table 1). A modified PfRH5 antigen of the invention may additionally comprise at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 30, at least 40, at least 50, at least 60 or more of the listed amino acid substitutions, or any combination thereof.

As described in detail herein, the present invention provides modified PfRH5 antigens with improved stability and expression profiles relative to the corresponding unmodified PfRH5 antigens, whilst at the same time retaining the desirable immunogenic and other functional properties of the unmodified PfRH5 antigens. To this end, the present inventors have identified residues that should preferably be unchanged between the modified PfRH5 antigens of the invention and the corresponding unmodified PfRH5 antigens. In particular, amino acid positions within 5 Å of the contact site between PfRH5 and basigin should remain unchanged, and/or amino acid positions within 5 Å of the contact site between PfRH5 and at least one anti-PfRH5 antibody with inhibitory activity, preferably the 9AD4 antibody or the QA1 antibody, more preferably both the 9AD4 and QA1 antibodies, should remain unchanged.

Accordingly, one or more of amino acid positions 147, 149, 193, 194, 196, 197, 198, 200, 201, 202, 203, 204, 205, 206, 207, 209, 212, 213, 216, 222, 225, 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and/or 496, or any combination thereof, may be unchanged in a modified PfRH5 antigen relative to the corresponding unmodified PfRH5 antigen. Typically, at least two, at least three at least four, at least five, at least six, at least seven, at least eight, at least nine, at least ten, at least 11, at least 12, at least 13, at least 14, at least 15, at least 16, at least 17, at least 18, at least 19, at least 20, or more of these positions remain unchanged in a modified PfRH5 antigen of the invention relative to the corresponding unmodified PfRH5 antigen.

In some embodiments, amino acid positions 147, 149, 193, 196, 202, 205, 206, 209, 212, 213, 216, 327, 328, 331, 334, 335, 337, 338, 339, 340, 341, 342, 344 and 452 are unchanged relative to the corresponding unmodified PfRH5 antigen. In other embodiments, amino acid positions 194, 197, 200, 201, 202, 203, 204, 207, 222, 225, 226, 242, 243, 244, 245, 246, 247, 248, 249 and 456 are unchanged relative to the corresponding unmodified PfRH5 antigen. In yet other embodiments, amino acid positions 198, 250, 343, 345, 346, 347, 348, 349, 350, 352, 353, 357, 358, 362, 447, 448, 449, 451 and 496 are unchanged relative to the corresponding unmodified PfRH5 antigen. In preferred embodiments, all of amino acid positions 147, 149, 193, 194, 196, 197, 198, 200, 201, 202, 203, 204, 205, 206, 207, 209, 212, 213, 216, 222, 225, 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and 496 are unchanged relative to the corresponding unmodified PfRH5 antigen.

The modified PfRH5 antigens of the invention may be derived from any of the unmodified PfRH5 antigens disclosed herein. As a non-limiting example, a modified PfRH5 antigen of the invention may be derived from an unmodified PfRH5 antigen selected from SEQ ID NOs: 1 to 14 as described herein, or a an unmodified PfRH5 antigen which exhibit at least 90%, 91%, 92%, 93%, 94%, 95%, 96%, 97% 98%, 99% or more identity with any one of SEQ ID NOs: 1 to 14. In preferred embodiments, a modified PfRH5 antigen of the invention is derived from an unmodified PfRH5 antigen of the invention selected from SEQ ID NOs: 7 to 14, or a variant thereof which exhibits at least 90% sequence identity with any one of SEQ ID NOs: 7 to 14, as defined herein. In particularly preferred embodiments, a modified PfRH5 antigen of the invention is derived an unmodified PfRH5 antigen of the invention selected from SEQ ID NOs: 7 to 10, or a variant thereof which exhibits at least 90% sequence identity with any one of SEQ ID NOs: 7 to 10, as defined herein. Any and all modifications described herein in relation to a modified PfRH5 antigen of the invention may be made in the context of any unmodified PfRH5 antigen, such as any of SEQ ID NOs: 1 to 14, preferably any of SEQ ID NOs: 7 to 14, even more preferably any of SEQ ID NOs: 7 to 10. A non-limiting exemplary modified PfRH5 antigens of the invention may comprise or consist of the amino acid sequence of any one of SEQ ID NOs: 15 to 56, with SEQ ID NOs: 21 to 28, 35 to 42 and 49 to 56 being preferred, and SEQ ID NOs: 21 to 28 being particularly preferred.

The modified PfRH5 antigens of the invention are typically more stable than the corresponding PfRH5 antigens of the invention. The term stability as used herein encompasses thermal stability, pH stability, stability in the presence of denaturants, and other forms of stability unless stated to the contrary. Any conventional method known in the art may be used to determine stability.

In particular, the modified PfRH5 antigens of the present invention have increased heat stability (also known as thermal stability). Thermal stability is the ability of a substance to resist an irreversible change in its chemical or physical structure. For proteins, such as PfRH5, thermal stability is the ability to resist denaturation with increasing temperature. Standard techniques are known in the art, including but not limited to circular dichroism, differential scanning calorimetry and surface plasmon resonance, and can be used to quantify thermal stability according to the present invention. Typically the modified PfRH5 antigens of the invention have an increase in thermal stability of at least 2° C., at least 3° C., at least 4° C., at least 5° C., at least 6° C., at least 7° C., at least 8° C., at least 9° C., at least 10° C., at least 11° C., at least 12° C., at least 13° C., at least 14° C., at least 15° C., at least 16° C., at least 17° C., at least 18° C., at least 19° C., at least 20° C., at least 25° C., at least 30° C., or more. Preferably a modified PfRH5 antigen of the invention will have an increase in thermal stability of at least 10° C., more preferably at least 15° C., even more preferably at least a 20° C., relative to the corresponding unmodified PfRH5 antigen.

The modified PfRH5 antigens of the invention typically have an improved expression profile compared with the corresponding PfRH5 antigens of the invention. An improved expression profile means that the modified PfRH5 antigen of the invention is expressed at higher levels, and/or will express in host systems in which the corresponding unmodified PfRH5 antigen will not be expressed. Preferably, the modified PfRH5 antigens of the invention express at both higher levels than the corresponding unmodified PfRH5 antigen, and will also express in additional host systems.

Typically a modified PfRH5 antigen of the invention will be expressed at least two-fold, at least three-fold, at least four-fold, at least five-fold, at least six-fold, at least seven-fold, at least eight-fold, at least nine-fold, at least ten-fold, at least 15-fold, at least 20-fold, at least 25-fold, at least 30-fold, at least 35-fold, at least 40-fold, at least 45-fold, at least 50-fold or more, relative to the expression level of the corresponding unmodified PfRH5 antigen in any given expression system, including each of those individualised herein. Preferably a modified PfRH5 antigen of the invention will be expressed at least two-fold, more preferably at least three-fold, even more preferably at least four-fold, relative to the corresponding unmodified PfRH5 antigen in any given expression system, including each of those individualised herein.

In some embodiments, a modified PfRH5 antigen of the invention will be expressed at a level of at least 0.5 mg/litre, at least 0.6 mg/litre, at least 0.7 mg/litre, at least 0.8 mg/litre, at least 0.9 mg/litre, at least 1.0 mg/litre, at least 1.1 mg/litre, at least 1.2 mg/litre, at least 1.3 mg/litre, at least 1.4 mg/litre, at least 1.5 mg/litre, at least 1.6 mg/litre, at least 1.7 mg/litre, at least 1.8 mg/litre, at least 1.9 mg litre, at least 2.0 mg/litre, at least 2.25 mg/litre, at least 2.5 mg/litre, at least 2.75 mg/litre, at least 3.0 mg/litre, at least 4 mg/litre, at least 5 mg/litre or more. Preferably a modified PfRH5 antigen of the invention will be expressed at least 1.0 mg/litre, more preferably at least 1.2 mg/litre, even more preferably at least 1.3 mg/litre, in any given expression system, including each of those individualised herein.

Typically using an expression system as defined herein and standard purification techniques known in the art, it is possible to obtain a purified modified PfRH5 antigen of the invention at a concentration of at least 0.5 mg/litre, at least 0.6 mg/litre, at least 0.7 mg/litre, at least 0.8 mg/litre, at least 0.9 mg/litre, at least 1.0 mg/litre, at least 1.1 mg/litre, at least 1.2 mg/litre, at least 1.3 mg/litre, at least 1.4 mg/litre, at least 1.5 mg/litre, at least 1.6 mg/litre, at least 1.7 mg/litre, at least 1.8 mg/litre, at least 1.9 mg/litre, at least 2.0 mg/litre, at least 2.25 mg/litre, at least 2.5 mg/litre, at least 2.75 mg/litre, at least 3.0 mg/litre or more. Preferably a purified modified PfRH5 antigen of the invention can be obtained of at least 1.0 mg/litre, more preferably at least 1.2 mg/litre, even more preferably at least 1.3 mg/litre, even more preferably at least 1.4 mg/litre or even more preferably at least 1.5 mg/litre in any given expression system, including each of those individualised herein.

A modified PfRH5 antigen of the invention may be expressed using any suitable host systems. Such a system may be a prokaryotic or a eukaryotic system. Examples of such systems are well-known in the art. Non-limiting examples of suitable host systems include Escherichia coli, Saccharomyces cerevisiae, Pichia pastoris, non-lytic insect cell expression systems such as Schneider 2 (S2) and Schneider 3 (S3) cells from Drosophila melanogaster and Sf9 and Sf21 cells from Spodoptera frugiperda, and mammalian expression systems such as CHO cells and human embryonic kidney (HEK/HEK293) cells. In a preferred embodiment, a modified PfRH5 antigen of the invention is expressed in functional, soluble form using a prokaryotic system, such as E. coli. The existing (unmodified) discontinuous fragment PfRH5 antigens cannot be expressed in functional form in such systems. Unmodified full-length PfRH5 has been reported in E. coli via insoluble inclusion bodies. Thus, this represents an advantage of the modified PfRH5 antigens of the invention over the unmodified PfRH5 antigens of the art.

Accordingly, the invention provides a host cell containing a recombinant expression vector which encodes for a modified PfRH5 antigen of the invention. In a preferred embodiment the host cell is an insect cell, preferably a Drosophila melanogaster cell, or an Escherichia coli cell.

A modified PfRH5 antigen of the invention will typically have the same functional features and immunogenicity as the corresponding unmodified PfRH5 antigen.

The term antigen as used herein refers to any peptide-based sequence that can be recognised by the immune system and/or that stimulates a cell-mediated immune response and/or stimulates the generation of antibodies. The modified PfRH5 antigens of the invention may be present in the form of a vaccine composition or vaccine formulation.

Typically the modified PfRH5 antigens of the invention bind to basigin (BSG), the red blood cell receptor for PfRH5. Binding of a modified PfRH5 antigen of the invention to basigin can be determined and/or quantified by any appropriate means. Standard methods for determining binding of a modified PfRH5 antigen of the invention to basigin, such as pull-down assays or surface plasmon resonance (SPR), are known in the art. In a preferred embodiment SPR is used to determine binding of modified PfRH5 antigens of the invention to basigin.

The modified PfRH5 antigens of the invention typically retain the same binding affinity for basigin as the corresponding unmodified PfRH5 antigen. In the context of the present invention, this may mean having a binding affinity for basigin of at least 80%, at least 85%, at least 90%, at least 95%, at least 99% or more of that of the corresponding unmodified PfRH5 antigen. Preferably the modified PfRH5 antigens of the invention have a binding affinity for basigin of at least 90%, at least 95%, at least 99% or more of that of the corresponding unmodified PfRH5 antigen.

In some embodiments, the modified PfRH5 antigens of the invention have a binding affinity for basigin greater than that of the corresponding unmodified PfRH5 antigen. For example, the modified PfRH5 antigens of the invention may have a binding affinity of at least 100%, at least 110%, at least 120%, or at least 150% or more of that of the corresponding unmodified PfRH5 antigen.

The binding affinity of a modified PfRH5 antigen of the invention for basigin may be quantified in terms of dissociation constant (K_{d}). K_{d} may be determined using any appropriate technique, but SPR is generally preferred in the context of the present invention. A PfRH5 fragment of the invention may bind to basigin with a K_{d} of less than 10 μM, less than 9 μM, less than 8 μM, less than 7 μM, less than 6 μM, less than 5 μM, less than 4 μM, less than 3 μM, less than 2 μM, less than 1.5 μM, less than 1 μM, less than 0.5 μM or less. Typically a PfRH5 fragment of the invention binds to basigin with a K_{d} of less 5 μM.

As discussed above, a modified PfRH5 antigen of the invention may have the same binding affinity for basigin as the corresponding unmodified PfRH5 antigen or a higher binding affinity for basigin as the corresponding unmodified PfRH5 antigen. Thus, a modified PfRH5 antigen of the invention may have the same K_{d} for binding to basigin as the corresponding unmodified PfRH5 antigen or a lower K_{d} for binding to basigin than the corresponding unmodified PfRH5 antigen respectively.

As described herein, the modified PfRH5 antigens of the invention raise antibodies that inhibit the growth of malarial parasites, i.e. Plasmodium parasites, preferably across a plurality of strains of blood-stage Plasmodium parasites, similarly to the corresponding unmodified PfRH5 antigens. In a more preferred embodiment, the modified PfRH5 antigens of the invention raise antibodies that inhibit the growth of Plasmodium falciparum parasites, and more preferably across a plurality of strains of blood-stage P. falciparum parasites. The effectiveness of the modified PfRH5 antigens of the invention may be quantified using any appropriate technique and measured in any appropriate units. For example, the effectiveness of the modified PfRH5 antigens of the invention may be given in terms of their growth inhibitory activity (GIA), half maximal effective concentration (EC₅₀), antibody titre stimulated (in terms of antibody units, AU) and/or EC₅₀ in terms of AU. The latter of these gives an indication of the quality of the antibody response stimulated by the modified PfRH5 antigen of the invention. Any appropriate technique may be used to determine the GIA, EC₅₀, AU or EC₅₀/AU. Exemplary techniques are described in the examples and conventional techniques are known in the art.

Typically, the modified PfRH5 antigens of the invention induce antibodies that have a growth inhibitory activity (GIA) of at least 30%, at least 40%, at least 50%, at least 60%, at least 70%, at least 80%, at least 90% or more against Plasmodium parasites. Typically this is comparable with the GIA induced by the corresponding unmodified PfRH5 antigens. In a preferred embodiment, the modified PfRH5 antigens of the invention induce antibodies that have a growth inhibitory activity (GIA) of at least 50%, at least 60%, at least 70%, at least 80%, at least 90% or more against Plasmodium parasites.

The growth inhibitory activity (GIA) may be measured at any appropriate concentration of the antibodies raised against the modified PfRH5 antigen, for example the GIA may be measured at 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml of purified IgG antibody. For example, the vaccine of the invention may comprise a modified PfRH5 antigen which will result in antibodies that give a GIA of least 20%, at least 30%, at least 50% and preferably at least 70% against the blood-stage Plasmodium parasite, at an IgG concentration of 10 mg/ml IgG, for example rabbit IgG.

Preferably the modified PfRH5 antigen of the invention is capable of inducing antibodies which exert similarly high levels of GIA against both the vaccine-homologous clone, 3D7, and against a vaccine-heterologous strain, FVO. Typically the total IgG induced by the modified PfRH5 antigen of the invention has an EC₅₀ which is comparable to total IgG raised against the corresponding unmodified PfRH5 antigen, and may be lower than the EC₅₀ against the corresponding unmodified PfRH5 antigen. The total IgG induced by the modified PfRH5 antigen of the invention preferably has an EC₅₀ significantly lower than that of the anti-PfAMA1 BG98 standard (Faber, B. W., et al., Infection and immunity, 2013; incorporated herein by reference). Typically a modified PfRH5 antigen of the invention induces IgG antibodies that have a total IgG EC₅₀ value of less than 10 mg/ml, less than 9 mg/ml, less than 8 mg/ml, less than 7 mg/ml, less than 6 mg/ml, less than 5 mg/ml, less than 4 mg/ml, less than 3 mg/ml, less than 2.5 mg/ml, less than 2 mg/ml, less than 1.5 mg/ml, less than 1 mg/ml, less than 0.5 mg/ml or less.

Typically the vaccine of the invention comprises a modified PfRH5 antigen of the invention which will raise antibodies that result in a GIA of at least 30%, at least 40%, at least 50%, at least 60%, at least 70%, at least 80%, at least 90% or more against the blood-stage Plasmodium parasite. In a preferred embodiment, the vaccine of the invention comprises a modified PfRH5 antigen of the invention which will raise antibodies that result in a GIA of at least 50% against the blood-stage Plasmodium parasite.

PfRH5 induces antibodies which are effective against genetically diverse strains of the Plasmodium parasite. This is likely to be of importance in achieving vaccine efficacy against the variety of strains circulating in the natural environment. Accordingly, in a preferred embodiment, the vaccine of the invention will raise antibodies that result in a GIA of at least 30% at least 40%, at least 50%, at least 60%, at least 70%, at least 80%, at least 90% or more against a plurality of genetic strains of the blood-stage Plasmodium parasite. In a preferred embodiment, the vaccine of the invention will raise antibodies that result in a GIA of at least 50% against a plurality of genetic strains of the blood-stage Plasmodium parasite.

Thus the vaccine of the invention can lead to improved outcomes after infection by P. falciparum and/or other species of the Plasmodium parasite. Monoclonal antibodies, DNA oligonucleotide aptatners, RNA oligonucleotide aptamers, and other engineered biopolymers against a modified PfRH5 antigen of the invention may also be able to replicate the activity of the vaccine-induced polyclonal antibodies described here. As a vaccine, modified PfRH5 antigens of the invention are likely amenable to expression by recombinant viral vectored vaccines, as well as nucleic acid-based vaccines such as RNA or DNA; and recombinant protein or virus-like particles (VLPs) expressed in mammalian expression systems or insect cell systems. It is also possible to express the modified PfRH5 antigens of the invention as proteins or VLPs in bacteria or yeast, as well as plant/algae systems.

The vaccine of the invention may comprise a combination of a modified PfRH5 antigen of the invention and one or more additional antigen(s) or fragment(s) thereof (preferably a PfAARP antigen or fragment thereof) that raise antibodies that give at least 70%, at least 80%, at least 85%, at least 90%, at least 95% or at least 99% GIA at a total antibody concentration of 10 mg/mL IgG, for example rabbit IgG. This combination is preferably equally effective against both the vaccine-homologous 3D7 clone and the vaccine-heterologous FVO strain.

The amount of antibody produced may be quantified using any appropriate method, with standard techniques being known in the art. For example, the amount of antibody produced may be measured by ELISA in terms of the serum IgG response induced by a modified PfRH5 antigen of the invention. The amount of antibody produced may be given in terms of arbitrary antibody units (AU). Typically, a modified PfRH5 antigen of the invention will produce an anti-PfRH5 antigen antibody response of at least 200 AU, at least 300 AU, at least 400 AU, at least 500 AU, at least 600 AU, at least 700 AU, at least 800 AU, at least 900 AU, at least 1000 AU at least 1100 AU, at least 1200 AU, at least 1300 AU, at least 1400 AU, at least 1500 AU or more.

The modified PfRH5 antigen of the invention may have a comparable immunogenicity relative to the corresponding unmodified PfRH5 antigen.

The immune response (or immunogenicity) to a modified PfRH5 antigen of the invention, particularly the antibody response, may be given as the half-maximal effective concentration in terms of the amount of antibody produced, i.e. EC₅₀/AU. This gives an indication of the quality of the immune response generated to a modified PfRH5 antigen. For example, a low EC₅₀ (i.e. effective response) but a high number of antibody units generated is less effective (and gives a higher EC₅₀/AU) than a low EC₅₀ with a low number of antibody units. This value thus indicates the quality of the antibody response by representing the functional anti-parasitic antibody activity (measured as the EC₅₀ in the assay of GIA) as a proportion of the total amount of anti-PfRH5 IgG antibody produced (measured by ELISA in AU). A more effective vaccine thus induces 50% GIA (the EC₅₀) with less antibody (lower AU).

Typically a modified PfRH5 antigen of the invention results in an EC₅₀/AU value of less than 1000, less than 900, less than 800, less than 700, less than 600, less than 500, less than 400, less than 300, less than 250, less than 200, less than 150, less than 100, less than 90, less than 80, less than 70, less than 60, less than 50, less than 40, less than 30, less than 20, less than 10 or less. In a preferred embodiment, a modified PfRH5 antigen of the invention results in an EC₅₀/AU value of less than 700, less than 600, less than 500, less than 400 or less.

A modified PfRH5 antigen of the invention typically elicits an equivalent immune response, particularly an equivalent antibody response, compared with the corresponding unmodified PfRH5 antigen. For example, a modified PfRH5 antigen of the invention may elicit antibodies with equivalent GIA, EC₅₀, and/or EC₅₀/AU than the corresponding unmodified PfRH5 antigen.

### Unmodified PfRH5 Antigens

Invasion of host red blood cells is an essential stage in the life cycle of the Plasmodium parasites and in development of the pathology of malaria. Central to invasion by all species are host-parasite interactions mediated by two parasite protein families, the reticulocyte-binding homologue (RH) proteins and the erythrocyte-binding like (EBL) proteins. In Plasmodium falciparum, just one member of these families, PfRH5, has been shown to be necessary for red blood cell invasion. In particular, PfRH5 is released onto the surfaces of infective P. falciparum merozoites, binding to human basigin in an interaction which is essential for erythrocyte invasion. Compared with other Plasmodium surface antigens, PfRH5 is remarkably conserved across field isolates. The present inventors have previously shown that antibodies targeting PfRH5 can block parasite invasion in vitro. In fact, antibodies that bind either PfRH5 or basigin show robust growth-inhibitory effects in vitro against all tested strains of P. falciparum. In addition, in a challenge trial, immunization with PfRH5-based vaccines protected Aotus monkeys against heterologous challenge with a virulent P. falciparum strain.

The modifications made to a modified PfRH5 antigen of the invention are described relative to the corresponding unmodified (native/wildtype) PfRH5 sequence. This may be a full-length PfRH5 protein sequence, or an antigenic fragment thereof. In addition, the unmodified PfRH5 antigen may itself contain some sequence alterations (e.g. between P. falciparum strains, etc.), provided said alterations have not been purposively derived by a PROSS method to improve the stability and expression profile of the unmodified antigen.

In one embodiment, the unmodified PfRH5 antigen is defined by SEQ ID NO: 1 or 2. Alternatively, the unmodified PfRH5 antigen may be the mature form of the antigen in which the N-terminal signal peptide has been removed. By way of example, the mature form may comprise or consist of amino acid residues 26 to 526 of SEQ ID NO: 1 or 2. The present invention embraces unmodified PfRH5 antigens which are fragments of said full-length PfRH5 proteins, which comprise or consist of 170 consecutive amino acid residues or more in length (e.g. at least 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510 or 520 consecutive amino acid residues in length). Such fragments have a common antigenic cross-reactivity with said unmodified (full-length) PfRH5 antigen. In one embodiment the unmodified PfRH5 antigens of the invention do not comprise amino acids from the N-terminal signal peptide. In one embodiment the unmodified PfRH5 antigens of the invention comprise amino acid residues 191 to 359 of SEQ ID NO: 1 or 2. In one embodiment the unmodified PfRH5 antigens of the invention comprise amino acid residues 31 to 174 of SEQ ID NO: 1 or 2. In one embodiment the unmodified PfRH5 antigens of the invention comprise amino acid residues 304 to 430 of SEQ ID NO: 1 or 2.

The unmodified PfRH5 antigen may have substitutions at amino acid residue 38 and/or at amino acid residue 214 of SEQ ID NO: 1 or 2, wherein the amino acid N is replaced by an amino acid other than N. In one embodiment the amino acid residue 38 and amino acid residue 214 are both replaced with Q.

The above-mentioned unmodified PfRH5 antigen thereof embraces variants exhibiting at least 80%, at least 85%, at least 90%, at least 91%, at least 92%, at least 93%, at least 94%, at least 95%, at least 96%, at least 97% at least 98%, at least 99% or more identity with SEQ ID NO: 1 or 2.

SEQ ID NOs: 1 and 2 consist of 526 amino acid residues. Variants of SEQ ID NO: 1 or 2 are encompassed as set out above and may additionally or alternatively include amino acid sequences with one or more amino acid substitutions, deletions or insertions. Substitutions are particularly envisaged, as are N- and C-terminal deletions. Substitutions include conservative substitutions. Thus, in one embodiment, a variant of SEQ ID NO: 1 or 2 comprises an N-terminal deletion of at least 1 consecutive amino acid residues (e.g. at least 30, 35, 40, 45 or 50 consecutive amino acid residues) in length. Thus, in one embodiment, a variant of SEQ ID NO: 1 or 2 comprises a C-terminal deletion of at least 1 consecutive amino acid residues (e.g. at least 5, 10, 15, 20, 25, 30, 35, 40, 45 or 50 consecutive amino acid residues) in length.

In one embodiment, the unmodified PfRH5 antigen includes the secretory signal from bovine tissue plasminogen activator, or may include another signal to direct the subcellular trafficking of the antigen. Alternatively, the antigen may be the mature form of the antigen in which the N-terminal signal peptide has been removed.

The above-mentioned unmodified PfRH5 antigens are described in detail in WO2012/114125 (herein incorporated by reference in its entirety).

The present inventors have previously solved the crystal structure of PfRH5 binding to basigin, its receptor on red blood cells, and have further identified the key amino acid residues in PfRH5 which contact basigin. The inventors have also solved for the first time the crystal structure of PfRH5 binding to a number of antibodies known to inhibit the invasion of red blood cells by Plasmodium parasites. Using this information, the present inventors have previously been able to design and develop PfRH5 fragments as improved malarial vaccine candidates. See WO2016/016651, which is herein incorporated by reference in its entirety. Any of the PfRH5 sequences disclosed in that document may be used as an unmodified PfRH5 antigen according to the present invention.

In particular, an unmodified PfRH5 antigen of present invention may be an antigenic fragment of PfRH5 which lack the flexible N-terminal region of the full length PfRH5 protein. Alternatively, the previously described discontinuous fragments of PfRH5, which lack the flexible loop region of full length PfRH5 as well as lacking the flexible N-terminal region may be used as unmodified PfRH5 antigens according to the present invention.

According to the present invention, an unmodified PfRH5 antigen may lack the flexible N-terminal region. Said flexible N-terminal region of PfRH5 typically comprises amino acid residues corresponding to amino acid residues 1 to 139 or 1 to 159 of SEQ ID NO: 1 or 2. Amino acid residues corresponding to amino acid residues 1 to 23 of SEQ ID NO: 1 or 2 are typically a signal peptide that is cleaved from the mature PfRH5 protein. As used herein, the term flexible N-terminal region may include or exclude the signal peptide. Thus, the term flexible N-terminal region may include the signal peptide and so refer to the amino acids corresponding to amino acid residues 1 to 139 or 1 to 159 of SEQ ID NO: 1 or 2. Alternatively, the term flexible N-terminal region may exclude the signal peptide and so refer to the amino acids corresponding to amino acid residues 24 to 139 or 24 to 159 of SEQ ID NO: 1 or 2. The present invention relates to unmodified PfRH5 antigens which lack the flexible N-terminal region of PfRH5, wherein the flexible N-terminal region of PfRH5 is as defined herein.

The present invention also relates to unmodified PfRH5 antigens which lack the flexible disordered central linker region of full-length PfRH5. Said flexible disordered central linker region of PfRH5 typically corresponds to amino acid residues 248 to 296 of SEQ ID NO: 1 or 2. The terms “flexible disordered central linker region”, “flexible central linker region” and “flexible central linker” are used interchangeable herein. The flexible central linker of PfRH5 as defined herein may comprise or consist of one of the recited sequences or variants thereof.

In embodiments where the unmodified PfRH5 antigen does not consist precisely of the sequence of SEQ ID NO: 1 or 2, i.e. a variant unmodified PfRH5 antigen (not to be confused with the modified PfRH5 antigens of the present invention), the flexible N-terminal region and the flexible central linker of said unmodified variant PfRH5 antigen will correspond to the N-terminal region and flexible central linker defined by reference to SEQ ID NO: 1 or 2. and may be easily identified using standard techniques. In particular, it is envisaged that the flexible N-terminal region and the flexible central linker of such a variant unmodified PfRH5 protein will have at least 60%, at least 70%, at least 80%, at least 85%, at least 90%, at least 95%, at least 99% or more sequence identity with the flexible N-terminal region or the flexible central linker respectively of the unmodified PfRH5 antigen as defined herein.

Typically, the unmodified PfRH5 antigen of the invention lacks the flexible N-terminal region of PfRH5 and/or the flexible central linker region of PfRH5. In a preferred embodiment, the unmodified PfRH5 antigen of the invention lacks both the flexible N-terminal region and the flexible central linker of PfRH5 as defined herein (see WO2016/016651, herein incorporated by reference).

The unmodified PfRH5 antigen of the invention may be a fragment of amino acid residues 140 to 526 of SEQ ID NO: 1 or 2, or a fragment of amino acid residues 160 to 526 of SEQ ID NO: 1 or 2. The unmodified PfRH5 antigen of the invention may be a fragment of an amino acid sequence having at least 70%, at least 80%, at least 90%, at least 95%, at least 99% or more sequence identity to amino acid residues 140 to 526 of SEQ ID NO: 1 or 2 or a fragment of an amino acid sequence having at least 70%, at least 80%, at least 90%, at least 95%, at least 99% or more sequence identity to amino acid residues 160 to 526 of SEQ ID NO: 1 or 2. In one embodiment, the unmodified PfRH5 antigen of the invention has the amino acid sequence of any one of SEQ ID NO: 3, 4, 5 or 6.

In a preferred embodiment, the unmodified PfRH5 antigens of the invention are discontinuous PfRH5 fragments. A discontinuous PfRH5 fragment is one which is lacking at least one region of continuous amino acids from within the full length PfRH5 protein, such that the discontinuous fragment has at least one gap or break in the full length PfRH5 sequence.

For example, full length PfRH5 comprises a flexible central linker as described herein, at amino acid residues corresponding to amino acid residues 248 to 296 of SEQ ID NO: 1 or 2. An unmodified PfRH5 antigen of the invention may lack this flexible central linker. A PfRH5 fragment comprising, for example, amino acid residues corresponding to amino acid residues 140 to 247 and 297 to 526 of SEQ ID NO: 1 or 2 as a single polypeptide is a discontinuous PfRH5 fragment, and hence an unmodified PfRH5 antigen according to the present invention. Another example of a unmodified PfRH5 antigen according to the present invention is a discontinuous PfRH5 fragment comprising amino acid residues corresponding to amino acid residues 160 to 247 and 297 to 526 of SEQ ID NO: 1 or 2 as a single polypeptide.

In one embodiment, an unmodified PfRH5 antigen of the invention lacks a flexible central linker as described herein, particularly a flexible central linker at amino acid residues corresponding to amino acid residues 248 to 296 of SEQ ID NO: 1 or 2. In a preferred embodiment, an unmodified PfRH5 antigen of the invention also lacks the flexible N-terminal region as described herein, particularly a flexible N-terminal region comprising amino acids corresponding to amino acids 1 to 139 or 1 to 159 of SEQ ID NO: 1 or 2. In a particularly preferred embodiment, an unmodified PfRH5 antigen of the present invention lacks both a flexible central linker as described herein and a flexible N-terminal region as described herein. Such a preferred unmodified PfRH5 antigen may lack a flexible central linker at amino acid residues corresponding to amino acid residues 248 to 296 of SEQ ID NO: 1 or 2 and a flexible N-terminal region comprising amino acids corresponding to amino acids 1 to 139 or 1 to 159 of SEQ ID NO: 1 or 2.

An unmodified PfRH5 antigen of the invention may have at least 70%, at least 80%, at least 85%, at least 90%, at least 95%, at least 99% or more sequence identity to SEQ ID NO: 5 or 6. Typically such an unmodified PfRH5 antigen of the invention has at least 90%, at least 95%, at least 99% or more sequence identity to any one of SEQ ID NO: 7 to 10.

Unmodified PfRH5 antigens of the present invention are typically greater than 20 amino acids in length. Unmodified PfRH5 antigens of the present invention may comprise or consist of at least 21, at least 30, at least 40, at least 50, at least 60, at least 70, at least 80, at least 90, at least 100, 110, 120, 130 140, 150, 160, 170, 180, 190, 200, 210, 220, 230, 240, 250, 260, 270, 280, 290, 300, 310, 320, 330, 340, 350, 360, 370, 380 or more amino acid residues in length. Unmodified PfRH5 antigens of the invention, including discontinuous PfRH5 fragments of the invention, may comprise regions of consecutive amino acids from the full length PfRH5 protein. For example, the unmodified PfRH5 antigens of the invention may comprise regions of at least 21, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220 or more consecutive amino acid residues in length. The unmodified PfRH5 antigens of the invention may be linear or branched, preferably linear. Any fragments provided as unmodified PfRH5 antigens of the invention have a common antigenic cross-reactivity with the full-length PfRH5 antigen.

The unmodified PfRH5 antigens of the invention may have substitutions at amino acid residues corresponding to amino acid residue 216 and/or amino acid residue 286 and/or amino acid residue 299 of SEQ ID NO: 1 or 2, wherein the amino acid T is replaced by an amino acid other than T. In one embodiment amino acid residues corresponding to amino acid residues 216, 286 and/or 299 of SEQ ID NO: 1 or 2 are replaced with A. Typically, amino acid residues corresponding to amino acid residues 216, 286 and 299 of SEQ ID NO: 1 or 2 are each replaced with A.

The unmodified PfRH5 antigens of the invention embrace fragments and/or variants of the full length PfRH5 protein, wherein said variants exhibit at least 90%, 91%, 92%, 93%, 94%, 95%, 96%, 97% 98%, 99% or more identity with SEQ ID NO: 1 or 2.

The unmodified PfRH5 antigens of the invention embrace the amino acid sequences of each of SEQ ID NOs: 1 to 14 as described herein. The unmodified PfRH5 antigens of the invention further embrace variants of any one of SEQ NOs: 1 to 14, wherein said variants exhibit at least 90%, 91%, 92%, 93%, 94%, 95%, 96%, 97% 98%, 99% or more identity with any one of SEQ ID NOs: 1 to 14. In preferred embodiments, the unmodified PfRH5 antigen of the invention is selected from SEQ ID NOs: 7 to 14, or a variant thereof which exhibits at least 90% sequence identity with any one of SEQ ID NOs: 7 to 14, as defined herein. In particularly preferred embodiments, the unmodified PfRH5 antigen of the invention is selected from SEQ ID NOs: 7 to 10, or a variant thereof which exhibits at least 90% sequence identity with any one of SEQ ID NOs: 7 to 10, as defined herein.

Conventional methods for determining amino acid sequence identity are known in the art. The terms “sequence identity” and “sequence homology” are considered synonymous in this specification.

By way of example, a polypeptide of interest may comprise an amino acid sequence having at least 70, 75, 80, 82, 84, 86, 88, 90, 92, 94, 96, 98, 99 or 100% amino acid sequence identity with the amino acid sequence of a reference polypeptide.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g. GAP, BESTFIT, FASTA or TFASTA), or BLAST and BLAST 2.0 algorithms.

The BLOSUM62 table shown below is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915-10919, 1992; incorporated herein by reference). Amino acids are indicated by the standard one-letter codes. The percent identity is calculated as:

$$\frac{Total number of identical matches}{[ \begin{matrix}length of the longer sequence plus the number of gaps \\ \begin{matrix}Introduced into the longer sequence in order to align the \\ two sequences\end{matrix}\end{matrix} ]} \times 100$$

BLOSUM62 table

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

In a homology comparison, the identity may exist over a region of the sequences that is at least 10 amino acid residues in length (e.g. at least 15, 20, 30, 40, 50, 75, 100, 150, 200, 250, 300, 350, 400, 450, 500 or 520 amino acid residues in length)—e.g. up to the entire length of the reference sequence.

Substantially homologous polypeptides have one or more amino acid substitutions, deletions, or additions. In many embodiments, those changes are of a minor nature, for example, involving only conservative amino acid substitutions. Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous polypeptides also encompass those comprising other substitutions that do not significantly affect the folding or activity of the polypeptide; small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

The modified PfRH5 antigens exemplified herein (namely PfRH5ΔN_{HS1}, PfRH5ΔN_{HS2} and PfRH5ΔN_{HS3}) are derived from the 7G8 strain of P. falciparum (i.e. the PfRH5 protein sequence of SEQ ID NO: 2). PfRH5 fragments, particularly fragments corresponding to PfRH5ΔN_{HS1}, PfRH5ΔN_{HS2} and PfRH5ΔN_{HS3}, derived from other P. falciparum strains are also encompassed by the present invention. In particular, the present invention encompasses modified PfRH5 antigens derived from the PfRH5 protein of the 3D7, 7G8 and FVO strains, preferably the PfRH5 of the 3D7 strain (SEQ ID NO: 1). The amino acid sequences of the PfRH5 proteins from the 7G8 and 3D7 strains are identical except for a single amino acid substitution: position 203 is a tyrosine (Y) in the 7G8 strain and a cysteine (C) in the 3D7 strain. Full length RH5 from the 3D7 strain has been shown to produce a higher quality antibody response than full length RH5 from the 7G8 strain. Accordingly, in one embodiment the modified PfRH5 antigens of the invention are derived from the corresponding unmodified PfRH5 antigen from the 3D7 strain.

The amino acid positions modified in a modified PfRH5 antigen of the invention may be defined relative to any of the unmodified PfRH5 antigens defined herein. As a non-limiting example, wherein a modified PfRH5 antigen of the invention comprises a modification at amino acid position 183, this encompasses a mutation at an amino acid residue corresponding to amino acid position 183 of any of the unmodified PfRH5 antigens defined herein. The position of a modified amino acid may be given relative to the amino acid sequence of the corresponding unmodified PfRH5 antigen. As a non-limiting example, if the modified PfRH5 antigen is a modified form of the full-length PfRH5 antigen of SEQ ID NO: 1, then amino acid position 183 of the modified PfRH5 antigen is the amino acid position corresponding to amino acid position 183 in SEQ ID NO: 1.

Typically, the position of a modified amino acid is given relative to the amino acid sequence of a full-length unmodified PfRH5 antigen, whether or not the corresponding unmodified PfRH5 antigen is a full-length PfRH5 antigen. As a non-limiting example, if the modified PfRH5 antigen is a modified form of a discontinuous PfRH5 fragment as described herein, then amino acid position 183 of the modified PfRH5 antigen may be the amino acid position corresponding to amino acid position 183 in any unmodified full-length PfRH5 antigen, such as SEQ ID NO: 1 or 2. As another non-limiting example, if the modified PfRH5 antigen is a modified form of an N-terminal-free PfRH5 antigen as described herein, then amino acid position 183 of the modified PfRH5 antigen may be the amino acid position corresponding to amino acid position 183 in any unmodified full-length PfRH5 antigen, such as SEQ ID NO: 1 or 2.

The unmodified PfRH5 antigens of the invention may additionally comprise a leader sequence, for example to assist in recombinant production and/or secretion. Any suitable leader sequence may be used, including conventional leader sequences known in the art. Suitable leader sequences include Bip leader sequences, which are commonly used in the art to aid secretion from insect cells and human tissue plasminogen activator leader sequence (tPA), which is routinely used in viral and DNA based vaccines and for protein vaccines to aid secretion from mammalian cell expression platforms.

The PfRH5 antigens of the invention may additionally comprise an N- or C-terminal tag, for example to assist in recombinant production and/or purification. Any N- or C-terminal tag may be used, including conventional tags known in the art. Suitable tags sequences include C-terminal hexa-histidine tags and the “C-tag” (the four amino acids EPEA at the C-terminus), which are commonly used in the art to aid purification from heterologous expression systems, e.g. insect cells, mammalian cells, bacteria, or yeast. Other examples of suitable tags include GST and MBP tags, or any other conventional tag which may be used to facilitate increased expression of a PfRH5 antigen. In other embodiments, the PfRH5 antigens of the invention are purified from heterologous expression systems without the need to use a purification tag.

The unmodified PfRH5 antigens of the invention may comprise a leader sequence and/or a tag as defined herein. Typically, the unmodified PfRH5 antigens of the invention comprise both a leader sequence and a C-terminal tag. For example, the unmodified PfRH5 antigens of the invention may comprise a Bip leader sequence and a C-terminal hexa-histidine tag. Such unmodified PfRH5 fragments of the invention may have at least 70%, at least 80%, at least 85%, at least 90%, at least 95%, at least 99% or more sequence identity with any one of SEQ ID NOs: 11 to 14.

As described herein, the modifications made to a modified PfRH5 antigen of the invention are described relative to the corresponding unmodified (native/wildtype) PfRH5 sequence. As a non-limiting example, in the case where the modified PfRH5 antigen is a modified form of full-length PfRH5, the corresponding unmodified PfRH5 antigen is the unmodified full-length PfRH5 sequence. As another non-limiting example, in the case where a modified PfRH5 antigen of the invention is a modified form of a PfRH5 fragment lacking the wildtype PfRH5 N-terminal signal sequence, the corresponding unmodified PfRH5 antigen is the unmodified PfRH5 fragment lacking the N-terminal signal sequence. As another non-limiting example, in the case where a modified PfRH5 antigen of the invention is a modified form of the discontinuous PfRH5 fragment PfRH5ΔNL, the corresponding unmodified PfRH5 antigen is unmodified sequence of the discontinuous PfRH5 fragment PfRH5ΔNL.

The corresponding unmodified PfRH5 antigen is typically of the same strain as the modified PfRH5 antigen of the invention. As a non-limiting example, if a modified PfRH5 antigen of the invention is based on the full-length PfRH5 sequence from the 3D7 strain, the corresponding unmodified PfRH5 antigen is the full-length unmodified PfRH5 sequence from the 3D7 strain. As another non-limiting example, if a modified PfRH5 antigen of the invention is based on the discontinuous PfRH5 fragment PfRH5ΔNL of the 3D7 strain, then the corresponding unmodified PfRH5 antigen is unmodified sequence of the discontinuous PfRH5 fragment PfRH5ΔNL of the 3D7 strain.

### Combinations of Antigens

The present inventors have also found that even greater efficacy can be achieved through combining PfRH5 with one or more of other P. falciparum antigens. GIA assays involving such combinations have demonstrated an effect which is greater than the sum of inhibition with individual antibodies, i.e. a synergistic effect. This was found to be the case even though other members of the PfRH family do not appear to be particularly effective in the GIA assay.

Accordingly, a modified PfRH5 antigen of the invention may be used in combination with one or more additional malarial antigen(s), or fragment thereof, including malarial antigens already known in the art.

For example, the present invention provides a modified Reticulocyte-binding protein Homologue 5 (PfRH5) antigen of the invention in combination with one or more antigens selected from the group consisting of PfAMA1, PfEBA175, PfRH1, PfRH2a, PfRH2b or PfRH4, PfCyRPA, PfRIPR, PfRH113 or PfAARP, or a fragment thereof. P. falciparum apical asparagine rich protein (PfAARP) is encoded by the P. falciparum clone 3D7 gene PF3D7_0423400 (previously known as MAL4P1.216 or PFD1105w). In particular, the present invention provides the modified PfRH5 antigen of the invention together with one or more of the PfEBA175, PfRH1, PfRH2a, PfRH2b, PfRH4, PfCyRPA, PfRIPR, PfP113 or PfAARP antigens, or a fragment thereof.

A particularly preferred embodiment includes the modified PfRH5 antigen of the invention together with a PfAARP antigen or fragment thereof. Such a combination may provide >90% GIA at a total antibody concentration of 0.625 mg/mL mouse IgG. Such a combination may be equally effective against both the vaccine-homologous 3D7 clone and the vaccine-heterologous FVO strain. One or more additional malarial antigen(s) can be used in combination with the modified PfRH5 antigen and PfAARP (or fragment) combination.

In one embodiment, the antigens or fragments thereof are present in the form of a vaccine formulation.

The combination of the invention may be present in a single vaccine product capable of inducing antibodies against both the modified PfRH5 antigen and the one or more additional antigen or fragment thereof. Alternatively the combination of the invention can be effected by mixing two separate recombinant protein vaccines (Pichyangkul, S., et al., Vaccine, 2009. 28(2): p. 452-62; and Ellis, R. D., et al., PLoS One. 2012. 7(10): p. e46094; both of which are incorporated herein by reference), or by co-delivering the modified PfRH5 antigen and one or more additional antigens or fragments thereof using vaccine platforms such as particle-based protein vaccine delivery (Bachmann, M. F., et al., Nat Rev Immunol, 2010. 10(11): p. 787-96; incorporated herein by reference), or virus-like particles (VLP), or by fusing or conjugating the modified PfRH5 antigen and the one or more additional antigen or fragment thereof to a construct or constructs that allow for particle formation and/or enhanced immunogenicity (Spencer, A. J., et al., PLoS One, 2012. 7(3): p. e33555; and Wu, Y., et al., Proc Natl Acad. Sci U S A, 2006. 103(48): p. 18243-8; both of which are incorporated herein by reference). In one embodiment, the modified PfRH5 antigen and the one or more additional antigen or fragment thereof may be delivered as a fusion protein (Biswas, S., et al., PLoS One, 2011. 6(6): p. e20977; incorporated herein by reference). Additionally or alternatively, the modified PfRH5 antigen and the one or more additional antigen or fragment thereof may be delivered using a mixture of viral vectors expressing the individual antigens (Forbes, E. K., et al., J Immunol, 2011. 187(7): p. 3738-50; and Sheehy, S. H., et al., Mol Ther, 2012. 20(12): p. 2355-68; both of which are incorporated herein by reference), or viral vectors co-expressing both the modified PfRH5 antigen and the one or more additional antigen or fragment thereof. Where the modified PfRH5 antigen and the one or more additional antigen or fragment thereof are co-expressed, this may be in the form of a fusion protein (Porter, D. W., et al., Vaccine, 2011. 29(43): p. 7514-22; incorporated herein by reference), or the modified PfRH5 antigen and the one or more additional antigen or fragment thereof expressed as separate transcripts under the control of separate promoters (Bruder, J. T., et al., Vaccine, 2010. 28(18): p. 3201-10; and Tine, J. A., et al., Infect Immun, 1996. 64(9): p. 3833-44; both of which are incorporated herein by reference), or the modified PfRH5 antigen and the one or more additional antigen or fragment thereof translated as a single polypeptide which undergoes cleavage to yield two separate antigens (Ibrahimi, A., et al., Hum Gene Ther, 2009. 20(8): p. 845-60; incorporated herein by reference).

### Vectors and Plasmids

The present invention provides a vector that expresses a modified PfRH5 antigen of the invention. Typically the vector is present in the form of a vaccine formulation.

The present invention further provides a vector that expresses a modified PfRH5 antigen of the invention, and one or more antigens selected from the group consisting of PfAMA1, PfEBA175, PfRH1, PfRH2a, PfRH2b, PfRH4, PfCyRPA, PfRIPR, PfP113 or PfAARP or a fragment thereof. In another aspect, the present invention provides a vector that expresses a modified PfRH5 antigen of the invention, together with a further vector that expresses one or more antigens selected from the group consisting of PfAMA1, PfEBA175, PfRH1, PfRH2a, PfRH2b, PfRH4, PfCyRPA, PfRIPR, PfP113, or PfAARP, or a fragment thereof. Preferred embodiments include a vector or vectors which express a modified PfRH5 antigen of the invention together with one or more of the PfEBA175, PfRH1, PfRH2a, PfRH2b, PfRH4, PfCyRPA, PfRIPR, PfP113 or PfAARP antigens, or a fragment thereof. The vector or vectors may be present in the form of a vaccine formulation.

The vector may be a viral vector. Such a viral vector may be an adenovirus (of a human serotype such as AdHu5, a simian serotype such as ChAd63, ChAdOX1 or ChAdOX2, or another form) or poxvirus vector (such as a modified vaccinia Ankara (MVA)). ChAdOX1 and ChAdOX2 are disclosed in WO2012/172277. ChAdOX2 is a BAC-derived and E4 modified AdC68-based viral vector.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, modified vaccinia Ankara (MVA) can be grown in CEF cells. In one embodiment, the vector is selected from a human or simian adenovirus or a poxvirus vector.

Typically, the viral vector is incapable of causing a significant infection in an animal subject, typically in a mammalian subject such as a human or other primate.

The invention further provides a DNA vector that expresses a modified PfRH5 antigen of the invention, such as a plasmid-based DNA vaccine. In one embodiment the DNA vector is capable of expression in a mammalian cell expression system, such as an immunised cell. The vector may be suitable for expression in a bacterial and/or insect host cell or expression system, such as any of those exemplified herein. A non-limiting example of a suitable expression vector is a pET15b vector, which may be optionally modified to encode an N-terminal tag, such as a hexa-histidine tag and/or a protease cleavage site, such as a TEV protease cleavage site.

The vector may be a RNA vector, such as a self-amplifying RNA vaccine (Geall, A. J. et al., Proc Natl Acad Sci USA 2012; 109(36) pp. 14604-9; incorporated herein by reference).

The present invention also provides virus-like particles (VLP) and/or fusion proteins comprising a modified PfRH5 antigen of the invention, as described herein. Methods for generating VLPs are known in the art (see, for example, Brune et al. Sci. Rep. (2016), 19(6):19234, which is incorporated by reference in its entirety) and can readily be applied to the present invention. References herein to vectors of the invention may apply equally to VLP and/or fusion proteins of the invention.

### Antibodies and Other Binding Compounds

As set out above, PfRH5 is a component of the mechanism by which the Plasmodium parasite invades RBCs. Compounds that specifically bind to PfRH5 inhibit this process and prevent the invasion of RBCs.

Accordingly, the present invention also provides binding compounds to a modified Reticulocyte-binding protein Homologue 5 (PfRH5) antigen of the invention.

The present invention also provides binding compounds to a modified PfRH5 antigen of the invention, in combination with binding compounds to any of PfAMA1, PfEBA175, PfRH1, PfRH2a, PfRH2b, PfRH4, PfRIPR, PfCyRPA, PfP113 or PfAARP, or fragments thereof. Particularly preferred embodiments include binding compounds to a modified PfRH5 antigen of the invention in combination with binding compounds to one or more of the PfEBA175, PfRH1, PfRH2a, PfRH2b, PfRH4, PfRIPR, PfCyRPA, PfP113 or PfAARP antigens or a fragment thereof.

The binding compound may be an antibody, such as a monoclonal antibody or polyclonal antibody. The binding compound may be an antigen-binding fragment of a monoclonal or polyclonal antibody, or a peptide which binds to a PfRH5 fragment of the invention with specificity. The antibody may be a Fab, F(ab′)2, Fv, scFv, Fd or dAb.

In another embodiment, the binding compound may be an oligonucleotide aptamer. The aptamer may bind to a modified PfRH5 antigen of the invention. The aptamer may specifically bind to the modified PfRH5 antigen or a fragment thereof.

Aptamers to PfRH5 may inhibit Plasmodium parasite growth in a GIA assay. Such aptamers can be found by known methods (e.g. as set out in D. H. J. Bunka, P. G. Stockley, Nature Reviews Microbiology 4, 588 (2006)). The aptamer may be optimised to render it suitable for therapeutic use, e.g. it may be conjugated to a monoclonal antibody to modify its pharmacokinetics (e.g. half-life and biodistribution) and/or recruit Fc-dependent immune functions.

The binding compound of the invention may be used in combination with a binding compound to one or more additional malarial antigen(s), including malarial antigens already known in the art. In a preferred embodiment, the present invention relates to the combination of a binding compound to a modified PfRH5 antigen of the invention with a binding compound to the PfAARP antigen or fragment thereof. One or more binding compound(s) to one or more additional malarial antigens can be used together with the combination of a binding compound to a modified PfRH5 antigen and the binding compound to PfAARP (or fragment).

Typically the binding compounds of the invention are specific for a modified PfRH5 antigen of the invention. By specific, it will be understood that a binding compound binds to the molecule of interest, in this case a modified PfRH5 antigen of the invention, with no significant cross-reactivity to any other molecule, particularly any other nucleic acid. For example, a binding compound or antibody that is specific for a modified PfRH5 antigen of the invention will show no significant cross-reactivity with human neutrophil elastase. Cross-reactivity may be assessed by any suitable method. Cross-reactivity of a binding compound (e.g. antibody) for a modified PfRH5 antigen with a molecule other than the PfRH5 fragment may be considered significant if the binding compound (e.g. antibody) binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to the modified PfRH5 antigen. A binding compound that is specific for a modified PfRH5 antigen may bind to another molecule such as human neutrophil elastase at less than 90%, 85%, 80%, 75%, 70%, 65%, 60%, 55%, 50%, 45%, 40%, 35%, 30%, 25% or 20% the strength that it binds to the modified PfRH5 antigen. Preferably, the binding compound (e.g. antibody) binds to the other molecule at less than 20%, less than 15%, less than 10% or less than 5%, less than 2% or less than 1% the strength that it binds to the modified PfRH5 antigen.

Typically the binding compounds of the invention are specific for a modified PfRH5 antigen of the invention, in that they do not binding to the unmodified form of the corresponding PfRH5 antigen, whether the unmodified antigen is a full-length PfRH5, a fragment of PfRH5 comprising the flexible N-terminal region, or a discontinuous fragment of PfRH5 as described herein.

### Therapeutic Indications

The present invention also provides a method of stimulating or inducing an immune response in a subject comprising administering to the subject a modified PfRH5 antigen of the invention, or vector of the invention, or a binding compound of the invention (as described above).

Thus, in one embodiment, the method of stimulating or inducing an immune response in a subject comprises administering a modified PfRH5 antigen of the invention, or a vector of the invention, or a binding compound of the invention (as described above) to a subject.

In the context of the therapeutic uses and methods, a “subject” is any animal subject that would benefit from stimulation or induction of an immune response against a Plasmodium parasite. Typical animal subjects are mammals, such as primates, for example, humans.

Thus, the present invention provides a method for treating or preventing alaria.

The present invention also provides a modified PfRH5 antigen of the invention for use in prevention or treatment of malaria. Said modified PfRH5 antigen may be in the form of a recombinant protein, a protein particle, a virus-like particle, a fusion protein, or a combination thereof as described herein.

The present invention further provides a modified PfRH5 antigen of the invention, and one or more further antigens selected from the group consisting of PfEBA175, PfRH1, PfRH2a, PfRH2b, PfRH4, PfRIPR, PfCyRPA, PfP113 or PfAARP, or a fragment thereof; for use in prevention or treatment of malaria. In a preferred embodiment, the present invention provides a modified PfRH5 antigen of the invention, and a PfAARP antigen or a fragment thereof; for use in prevention or treatment of malaria.

The present invention provides the vectors as described herein for use in the prevention or treatment of malaria.

The present invention further provides the binding compounds as described herein for use in the prevention or treatment of malaria.

The present invention provides the use of a modified PfRH5 antigen of the invention, vector, or binding compound of the invention (as described above) for use either alone or in combination in the prevention or treatment of malaria.

Additionally, the present invention provides the use of a modified PfRH5 antigen of the invention, vector, or binding compound of the invention (as described above), in the manufacture of a medicament for the prevention or treatment of malaria.

In one embodiment, the method for treating or preventing malaria comprises administering a therapeutically effective amount of a modified PfRH5 antigen of the invention, or binding compound, or a vector, of the invention (as described above), either alone or in combination, to a subject.

As used herein, the term “treatment” or “treating” embraces therapeutic or preventative/prophylactic measures, and includes post-infection therapy and amelioration of malaria.

As used herein, the term “preventing” includes preventing the initiation of malaria and/or reducing the severity or intensity of malaria. The term “preventing” includes inducing or providing protective immunity against malaria. Immunity to malaria may be quantified using any appropriate technique, examples of which are known in the art.

A modified PfRH5 antigen of the invention, or binding compound, or a vector, of the invention (as described above) may be administered to a subject (typically a mammalian subject such as a human or other primate) already having malaria, a condition or symptoms associated with malaria, to treat or prevent malaria. For example, the subject may be suspected of having come in contact with Plasmodium parasite, or has had known contact with Plasmodium parasite, but is not yet showing symptoms of exposure.

When administered to a subject (e.g. a mammal such as a human or other primate) that already has malaria, or is showing symptoms associated with Plasmodium parasite infection, a modified PfRH5 antigen of the invention, or binding compound, or a vector, of the invention (as described above) can cure, delay, reduce the severity of, or ameliorate one or more symptoms, and/or prolong the survival of a subject beyond that expected in the absence of such treatment.

Alternatively, a modified PfRH5 antigen of the invention, or binding compound, or a vector, of the invention (as described above) may be administered to a subject (e.g. a mammal such as a human or other primate) who ultimately may be infected with Plasmodium parasite, in order to prevent, cure, delay, reduce the severity of, or ameliorate one or more symptoms of malaria, or in order to prolong the survival of a subject beyond that expected in the absence of such treatment, or to help prevent that subject from transmitting malaria.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects.

The present invention provides vaccine compositions comprising any of the modified PfRH5 antigens of the invention (described herein). Said vaccine compositions may further comprise one or more additional malarial antigens as described herein, and/or any further components as described herein.

A modified PfRH5 antigen of the invention, or a vector of the invention (as described above) can be employed as vaccines. Accordingly, the present invention provides a vaccine composition comprising a modified PfRH5 antigen of the invention.

A vaccine composition of the invention comprising a modified PfRH5 antigen of the invention may further comprise one or more additional antigens selected from the group consisting of PfAMA1, PfEBA175, PfRH1, PfRH2a, PfRH2b, PfRH4, PfRIPR, PfCyRPA PfP113 or PfAARP, or a fragment thereof. For example, the present invention provides a vaccine composition comprising a modified PfRH5 antigen of the invention in combination with one or more of the PfEBA175, PfRH1, PfRH2a, PfRH2b, PfRH4, PfRIPR, PfCyRPA, PfP113 or PfAARP antigens or a fragment thereof. In a preferred embodiment, the present invention provides a vaccine composition comprising a modified PfRH5 antigen of the invention in combination with a PfAARP antigen or a fragment thereof.

The present invention provides a vaccine composition comprising a vector that expresses a modified PfRH5 antigen of the invention. The vector of such a vaccine composition may further express one or more additional antigens selected from the group consisting of PfAMA1, PfEBA175, PfRH1, PfRH2a, PfRH2b, PfRH4, PfRIPR, PfCyRPA PfP113 or PfAARP, or a fragment thereof. Alternatively, the present invention provides a vaccine composition comprising a vector that expresses a modified PfRH5 antigen of the invention, together with a vector that expresses one or more further antigens selected from the group consisting of PfAMA1, PfEBA175, PfRH1, PfRH2a, PfRH2b, PfRH4 PfRIPR, PfCyRPA PfP113 or PfAARP, or a fragment thereof. For example, the present invention provides a vaccine composition comprising a vector or vectors that express a modified PfRH5 antigen of the invention in combination with one or more of the PfEBA175, PfRH1, PfRH2a, PfRH2b, PfRH4 PfRIPR, PfCyRPA PfP113 or PfAARP antigens, or a fragment thereof.

In a further aspect the present invention provides a vaccine composition comprising a modified PfRH5 antigen of the invention, optionally together with one or more additional antigens or fragments thereof (particularly PfAARP or a fragment thereof), where either or both the modified PfRH5 antigen and/or the one or more additional antigen or fragment thereof may be expressed as a virus like particle (VLP). Recombinant particulate vaccines are well known in the art. They may be, for example, either fusion proteins or proteins chemically conjugated to particles. Examples of fusion proteins are hepatitis B surface antigen fusions (e.g. as in the RTS,S malaria vaccine candidate), hepatitis B core antigen fusions, or Ty-virus like particles. Examples of chemical fusion particles are the Q-beta particles under development by the biotechnology company Cytos (Zurich, Switzerland) and as in Brune et al. Sci. Rep. (2016), 19(6):19234.

The present invention further provides a vaccine composition comprising a modified PfRH5 antigen of the invention, optionally together with one or more additional antigen or a fragment thereof (particularly PfAARP or a fragment thereof), where either or both the modified PfRH5 antigen and/or the one or more additional antigen or fragment thereof may be expressed as a soluble recombinant protein. Recombinant protein-based vaccines are well known in the art. They may be, for example, monomeric soluble proteins or soluble fusion proteins. Such proteins are typically administered or formulated in a vaccine adjuvant. Examples of protein-based vaccines are diphtheria and tetanus toxoids, or soluble malaria protein antigens such as the AMA1 protein vaccine candidates developed for blood-stage malaria (Spring, M. D., et al., PLoS ONE, 2009, 4(4): p. e5254; incorporated herein by reference).

A modified PfRH5 antigen of the invention and one or more additional antigen or fragment thereof (preferably a PfAARP antigen or fragment thereof) may be combined to provide a single vaccine product (as described above) capable of inducing antibodies against both antigens, e.g. by mixing two separate recombinant protein vaccines, or by co-delivering the antigens using vaccine platforms such as particle-based protein vaccine delivery, or using a fusion of the two antigens; or by using a mixture of viral vectors expressing the individual antigens, or viral vectors co-expressing both antigens.

As used, herein, a “vaccine” is a formulation that, when administered to an animal subject such as a mammal (e.g. a human or other primate) stimulates a protective immune response against Plasmodium parasitic infection. The immune response may be a humoral and/or cell-mediated immune response. A vaccine of the invention can be used, for example, to protect a subject from the effects of P. falciparum infection (i.e. malaria).

The lack of polymorphism at the PfRH5 locus (five non-synonymous SNP across its entire length in circulating P. falciparum parasites) suggest either a lack of substantial immune pressure, or a high degree of functional constraint that prevents mutations from freely occurring. This property makes it highly likely that functional antibodies raised against a fragment of a single allele of PfRH5 according to the present invention will have broadly neutralising activity.

Thus, the PfRH5 fragments of the invention typically induce antibodies that provide a highly effective cross-strain GIA against the Plasmodium parasite. Thus, in one embodiment, a modified PfRH5 antigen of the invention provides protection (such as long term protection) against disease caused by Plasmodium parasites. Typically, a modified PfRH5 antigen of the invention provides an antibody response (e.g. a neutralising antibody response) to Plasmodium parasitic infection. The modified PfRH5 antigens, vaccine compositions, vectors, plasmids, antibodies and/or aptamers of the invention as described herein may be used to confer pre-erythrocytic or transmission-blocking protection against Plasmodium parasites.

### Pharmaceutical Compositions and Formulations

The term “vaccine” is herein used interchangeably with the terms “therapeutic/prophylactic composition”, “formulation” or “medicament”.

The vaccine of the invention (as defined above) can be combined or administered in addition to a pharmaceutically acceptable carrier. Alternatively or in addition the vaccine of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations vaccines) is generally by conventional routes e.g. intravenous, subcutaneous, intraperitoneal, or mucosal routes. The administration may be by parenteral injection, for example, a subcutaneous, intradermal or intramuscular injection. Formulations comprising neutralizing antibodies may be particularly suited to administration intravenously, intramuscularly, intradermally, or subcutaneously.

Accordingly, immunogenic compositions, therapeutic formulations, medicaments and prophylactic formulations (e.g. vaccines) of the invention are typically prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as a modified PfRH5 antigen of the invention) are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It is within the routine practice of a clinician to determine an effective amount of a vaccine composition of the invention. An effective amount is an amount sufficient to elicit a protective immune response against malaria. A clinician will also be able to determine appropriate dosage interval using routine skill.

### Method of Designing Modified PfRH5 Antigens

Many promising vaccine candidates from pathogenic viruses, bacteria, and parasites are unstable and cannot be produced cheaply for clinical use. For instance, P. falciparum RH5 is essential for erythrocyte invasion, is highly conserved among field isolates, and is able to elicit antibodies that protect in vitro and in an animal model, making it a leading malaria vaccine candidate. However, moderate thermal resistance and high production costs associated with insect-cell expression for unmodified PfRH5 antigens affect their applicability.

The inventors have previously developed stability-design algorithm, called PROSS, (see PCT/IL2016/050812 and Goldenzweig, A. et al. [Mol Cell, 2016, 63(2), pp. 337-46], which are herein incorporated by reference) that is effective in designing variants of challenging human enzymes with much improved thermal stability and increased bacterial expression levels, without affecting protein function. Briefly, the PROSS workflow comprises three stages. It first extracts sequences and analyses homologues of the target protein to contrast an alignment and general a statistical model of amino acid probabilities to identify, at each amino acid position, mutations that are most likely to occur through natural diversity of the protein family. Second, starting from a molecular structure of the target protein, Rosetta computational design simulations suggest a subset of these mutations, which are individually predicted to stabilize the unmodified (wildtype) protein. At the last step, Rosetta combinatorial sequence optimization is used to suggest several optimized design modifications, typically comprising >10 mutations each, with improved native-state energy. Any potentially destabilising mutations are excluded based on the calculated energy value (typically a cut off ΔΔG value is set as 0, or in some instances −0.45, with all possible mutations with a ΔΔG value above the cut off being excluded). In all steps, mutations that might affect the conformation of residues at the active site are eliminated. In this workflow, natural sequence diversity provides information on tolerated and possibly beneficial variations.

Using PROSS, the present inventors have designed three modified PfRH5 antigens for improved packing and surface polarity. The best, bearing 18 mutations relative to unmodified PfRH5, has been demonstrated to bind to basigin and elicit inhibitory antibodies. It also expresses in a folded form in bacteria and showed >10° C. higher thermal resistance than the corresponding unmodified PfRH5 antigen, proving its value as an immunogen for a new generation of vaccines against the malaria blood-stage. Thus, the inventors have demonstrated modified and improved PfRH5 antigens.

Accordingly, PROSS has been used to identify at least one stabilising mutation within the amino acid sequence of the PfRH5 antigen, by: (a) aligning an unmodified PfRH5 amino acid sequence against a non-redundant protein database; (b) identifying homologues to an unmodified PfRH5 amino acid sequence; (c) excluding homologues from genera other than Plasmodium; (d) excluding homologues with a certain degree of gaps compared with the unmodified PfRH5 sequence; and (e) processing the amino acid sequences of the non-excluded homologues via the PROSS methodology to identify stabilising mutations within the PfRH5 amino acid sequence. The application of PROSS used by the inventors is described in detail in the Examples.

Said application of PROSS may exclude mutations at amino acid positions within 10 Å, preferably within 5 Å of the contact sites of PfRH5 with basigin. Alternatively or in addition, the algorithm may exclude mutations at amino acid positions within 10 Å, preferably within 5 Å of the contact sites of PfRH5 with at least one anti-PfRH5 antibody, preferably the 9AD4 antibody, and/or the QA1 antibody, preferably both the 9AD4 and QA1 antibodies. Typically, said algorithm excludes mutations at amino acid positions within 10 Å, preferably within 5 Å of the contact sites of with basigin and mutations at amino acid positions within 10 Å, preferably within 5 Å of the contact sites of PfRH5 with at least one anti-PfRH5 antibody. In preferred embodiments, said algorithm excludes mutations at amino acid positions within 10 Å, preferably within 5 Å of the contact sites of PfRH5 with basigin and mutations at amino acid positions within 10 Å, preferably within 5 Å of the contact sites of PfRH5 with the 9AD4 antibody, and/or the QA1 antibody. More preferably, said algorithm excludes mutations at amino acid positions within 5 Å of the contact sites of PfRH5 with basigin and mutations at amino acid positions within 5 Å of the contact sites of PfRH5 with both the 9AD4 and QA1 antibodies.

The PROSS method may be used to identify one, two, three, four, five, six, seven, eight, nine, ten, 11, 12, 13, 14, 15, 16, 17, 18, 19, 20 or more mutations relative to the unmodified PfRH5 antigen that may be incorporated into a modified PfRH5 antigen of the invention as described herein.

Any unmodified PfRH5 antigen as described herein may be used in the application of PROSS as described herein. Typically the method uses the full-length unmodified PfRH5 antigen of SEQ ID NO: 1 or 2, any of SEQ ID NOs: 3 to 14, or variants thereof as defined herein. As a non-limiting example, the application of PROSS may use an unmodified discontinuous PfRH5 fragment as the unmodified PfRH5 antigen. Thus, in some preferred embodiments, the unmodified PfRH5 antigen used is selected from SEQ ID NOs: 7 to 14, or a valiant thereof which exhibits at least 90% sequence identity with any one of SEQ ID NOs: 7 to 14, as defined herein. In particularly preferred embodiments, the unmodified PfRH5 antigen of the invention is selected from SEQ ID NOs: 7 to 10, or a variant thereof which exhibits at least 90% sequence identity with any one of SEQ ID NOs: 7 to 10, as defined herein. In other words, in some particularly preferred embodiments, PROSS uses PfRH5ΔNL as the unmodified PfRH5 antigen.

The invention further provides a method of producing a modified PfRH5 antigen, comprising identifying at least one stabilising mutation using PROSS, and expressing the modified PfRH5 antigen in a host cell or expression system. Suitable host cells and expression systems are known in the art and examples of such are described herein. A modified PfRH5 antigen generated according to said method is typically a modified PfRH5 antigen as described herein. The invention further provides a modified PfRH5 antigen obtainable by a method of the invention.

### EXAMPLES

### Example 1

### Design of Stable Modified PfRH5 Antigens

### Homologous Sequences Collection and Filtering

The PfRh5 structure was downloaded from the Protein Data Bank (entry: 4WAT) and homologous sequences were collected using CSI-BLAST to search the non-redundant (nr) database in May 2015, with e-value <10⁻⁴, three iterations, a maximum of 500 sequences, and default values on all other parameters. Hits were clustered using cd-hit at 98% threshold and default parameters. Hits from genera other than Plasmodium were excluded. Hits were also excluded if their sequence identity to the query was lower than 15% or if they showed more than 1% gaps in the aligned segment.

Of the remaining sequences, two sets of hits were defined. A ‘strict’ set containing only hits sharing 19% sequence identity to the query or more, and a ‘permissive’ set containing all remaining hits (8 and 14 hits, respectively, including the query sequence). The ‘strict’ alignment contained the following UniProt entries: Q8IFM5 (PDB entry: 4WAT), A0A078K5N4, B4X6H6, K6VIX0, A0A060RXZ9, W7J6M4, A0A024WYW5, Q7YWE8; the ‘permissive’ alignment contained the entries in the ‘strict’ alignment and the following additional entries: I6QQT7, C11W27, I6RGY9, A5K940,A5K696, A0A060S1Z4, MUSCLE was used with default parameters to derive a multiple sequence alignment from each set of hits.

### PROSS Stability Design

The PROSS algorithm applied two filters to reduce the sequence space from the theoretical space of 20 amino acid options at each position (i.e., 20ⁿ where n is the number of amino acids), to a much smaller space enriched with amino acids predicted to be individually stabilizing.

The first filter was based on sequence analysis of homologues as described above. In the second filter, all amino acids passing the first filter were modelled structurally against wild type background (using the Rosetta software) and only amino acids predicted as stabilizing individually were kept. In principle mutations predicted as individually stabilizing are mutations with stability score (ΔΔG_{calc}) smaller than 0. The PROSS algorithm defined mutations as stabilising only if their stability score was ΔΔG_{calc}<−0.45 rosetta energy units (r.e.u). Amino acid options passing both filters defined the sequence space (Table 1) of the modified PfRH5 antigens of the invention.

TABLE 1
PfRH5 sequence space

| | Position | Sequence space | | | |
|---|---|---|---|---|---|
| | (numbering | (wild type | | | |
| | according to Protein | (unmodified) | | | |
| | DataBase ID | amino acid first | | | |
| No. | 4WAT) | from the left) | PfRH5ΔNL_{HS1} | PfRH5ΔNL_{HS2} | PfRH5ΔNL_{HS3} |
| 1. | 157 | I/L | L | | |
| 2. | 164 | L/F | | | |
| 3. | 171 | L/V | | | |
| 4. | 178 | H/Y | | | |
| 5. | 183 | D/E/Y | E | E | E |
| 6. | 188 | L/T/V | | | |
| 7. | 191 | N/I | | I | I |
| 8. | 192 | S/A | | A | A |
| 9. | 195 | H/Y | | | |
| 10. | 221 | K/I | | | |
| 11. | 230 | D/E | | | |
| 12. | 231 | L/F | | | |
| 13. | 233 | A/N/T/K | K | N | N |
| 14. | 234 | T/L | | | |
| 15. | 236 | K/H | | | H |
| 16. | 300 | F/Y | | | |
| 17. | 304 | M/I/F | F | | |
| 18. | 305 | D/N | | | |
| 19. | 308 | N/K | | | K |
| 20. | 309 | T/K | | | |
| 21. | 311 | K/I | | | |
| 22. | 312 | K/N | N | | |
| 23. | 314 | L/F/Y | F | | F |
| 24. | 315 | I/H/M | | | |
| 25. | 316 | K/N/Q | N | | |
| 26. | 330 | M/N | N | | |
| 27. | 336 | G/S | | | |
| 28. | 354 | N/P | | | |
| 29. | 365 | H/R | | | |
| 30. | 368 | I/M | | | |
| 31. | 369 | L/N | | N | N |
| 32. | 370 | S/E/K/N/A | A | | K |
| 33. | 381 | S/D/K/N | N | N | N |
| 34. | 384 | T/K | K | | K |
| 35. | 390 | S/A | | | |
| 36. | 391 | E/I | | | |
| 37. | 392 | L/D/K | K | D | D |
| 38. | 394 | L/I/V | | | |
| 39. | 395 | T/N/R | N | | R |
| 40. | 396 | N/K | | | |
| 41. | 398 | N/E/K/R | E | K | K |
| 42. | 401 | M/I | | | |
| 43. | 406 | Y/V | | | |
| 44. | 414 | H/I/L | | | |
| 45. | 422 | N/E | | | |
| 46. | 424 | I/F/M | | | |
| 47. | 428 | T/I | | | |
| 48. | 435 | T/I/Y | | | |
| 49. | 442 | I/F/K/Y | | | |
| 50. | 444 | L/D/E/N/Q | | | E |
| 51. | 445 | N/D/V | | D | D |
| 52. | 455 | L/F | | | |
| 53. | 458 | R/K | K | | |
| 54. | 463 | S/A/V | | A | A |
| 55. | 464 | N/K | K | K | K |
| 56. | 467 | S/A | A | A | A |
| 57. | 468 | L/I | | | |
| 58. | 470 | I/K/R | | R | R |
| 59. | 474 | H/D | | D | D |
| 60. | 479 | L/F | | | |
| 61. | 481 | N/K | | | |
| 62. | 485 | S/H/L/T | | | |
| 63. | 495 | H/N | | | N |
| 64. | 505 | F/L | L | | L |
| 65. | 511 | K/P | | P | P |

Options from this space were subjected to a final step in which the full protein was designed simultaneously (to derive optimal combinations for experimental testing). The three modified PfRH5 antigens tested were derived from the final step. However, other combinations of mutations from the sequence space could comprise RH5 stable variants and therefore they need to be protected in the patent.

To preserve the PfRh5 binding interface with its natural target basigin (Protein Data Bank entry: 4U0Q) and with two neutralizing antibodies, 9AD4 and QA1 (Protein Data Bank entries: 4U0R and 4U1G, respectively), 63 residues within 5 Å of all three interfaces were held fixed throughout all Rosetta simulations (see Table 2 below).

TABLE 2
Computational parameters used to generate
designed variants and lists of mutations.

| Designed | | | |
|---|---|---|---|
| modified | | ΔΔG_{calc} | |
| PfRH5 | Positions held | cutoffs^{a} | |
| antigen | fixed^{a,b} | (R.e.u.)^{c} | Mutations^{a} |
| PfRH5ΔNL_{HS1} | 147, 149, 193, 196, | −0.45 | I157L, D183E, A233K, |
| (strict MSA) | 202, 205, 206, 209, | (18) | M304F, K312N, L314F, |
| | 212, 213, 216, 327, | | K316N, M330N, S370A, |
| | 328, 331, 334, 335, | | S381N, T384K, L392K, |
| | 337, 338, 339, 340, | | T395N, N398E, R458K, |
| | 341, 342, 144, 452, | | N464K, S467A, F505L |
| PfRH5ΔNL_{HS3} | 456, 194, 197, 200, | −1.25 | D183E, N191I, S192A, |
| (permissive | 201, 202, 203, 204, | (15) | A233N, L369N, S381N, |
| MSA) | 207, 222, 225, 226, | | T392D, N398K, N445D, |
| | 242, 243, 244, 245, | | S463A, N464K, S467A, |
| | 246, 247, 248, 249, | | I470R, H474D, K511P |
| PfRH5ΔNL_{HS2} | 250, 346, 347, 349, | −0.75 | D183E, N191I, S192A, |
| (permissive | 350, 352, 337, 358, | (25) | A233N, K236H, N308K, |
| MSA) | 362, 447, 448, 449, | | L314F, L369N, S370K, |
| | 496, 198, 343, 345, | | S381N, T384K, T392D, |
| | 348, 353, 451 | | T395R, N398K, H414L, |
| | | | L444E, N445D, S463A, |
| | | | N464K, S467A, I470R, |
| | | | H474D, H495N, F505L |
| | | | K511P |

^{a}Numbering according to Protein Database (PDB) entry 4WAT.
^{b}Residues within 5 Å of either basigin, QA1 or 9AD4.
^{c}PROSS cutoff of designs that were selected for experimental validation. The cut-offs defined the subsets of individually stabilizing mutations. Each defined subset resulted in a single modified PfRH5 antigen. R.e.u, refers to Rosetta energy units. Figures in parentheses show the number of mutations relative to unmodified (wild-type) PfRH5ΔNL.

Two independent runs of the PROSS algorithm were carried out based on the ‘strict’ and ‘permissive’ alignments and the sequence spaces merged to generate the sequence space of Table 1. As a template structure (i.e. unmodified PfRH5 antigen), a version of RH5 with both the flexible N-terminus (residues 1-140) and a disordered loop (residues 248-296) removed (PfRH5ΔNL, previously described in WO20161016651) was used. Since PfRh5 is large (>400 amino acids), it was decided to experimentally test designs with 15 mutations or more. Using the ‘strict’ alignment, only one design had more than 15 mutations (PfRH5ΔNL_{HS1}) and using the ‘permissive’ alignment two designs were selected for experimental testing based on visual inspection (PfRH5ΔNL_{HS2} and PfRH5ΔNL_{HS3}) (Table 2).

### Design Model Analysis

Sequence and structural features of the modified PfRH5 antigen PfRH5ΔNL_{HS1} were compared to the sequence and structure of the unmodified (wild-type) PfRH5ΔNL antigen. Mutations were defined as improving helical propensity if they were in helical regions and if Rosetta energy calculations showed ΔΔG_{calc}<−0.15 Rosetta energy units for the energy term that accounts for sequence secondary-structure compatibility (p_aa_pp). Positions were defined as buried if they had >21 and >75 neighbouring non-hydrogen atoms within 10 Å and 12 Å, respectively, according to the Rosetta Features Reporter.

### Results and Discussion

PfRH5, presents an unusual challenge for sequence analysis. As of May 2015, sequences of PfRH5 from P. falciparum field isolates showed 99% sequence identity to one another, and only one orthologue, from P. reichenowi (with 66% sequence identity to PfRH5) was available.

Since the PfRH5 fold is considered unique and highly conserved in Plasmodium invasion proteins, these restrictions increase the likelihood that the sequences in the alignment belong to the same fold. As a safety measure two alignments were generated in CSI-BLAST, a ‘permissive’ alignment containing all the homologues described above (Table 2) and a ‘strict’ one at >18% sequence identity. These alignments had 14 and 8 sequences, respectively. As a template structure, a version of PfRH5 with both the flexible N-terminus (residues 1-140) and a disordered loop (residues 248-296) removed (PfRH5ΔNL) was used as the unmodified PfRH5 antigen, as this contains the structured region of RH5 and retains the capacity to bind to basigin and induce production of inhibitory antibodies.

The two alignments and the PfRH5ΔNL structure were then provided to the PROSS algorithm in two independent runs. To preserve the function and immunological efficacy of the designed RH5 variants mutations at amino acid positions within 5 Å of the contact sites of either basigin or two anti-PfRH5 inhibitory antibodies, 9AD4 and QA16,8 were not allowed (Table 2).

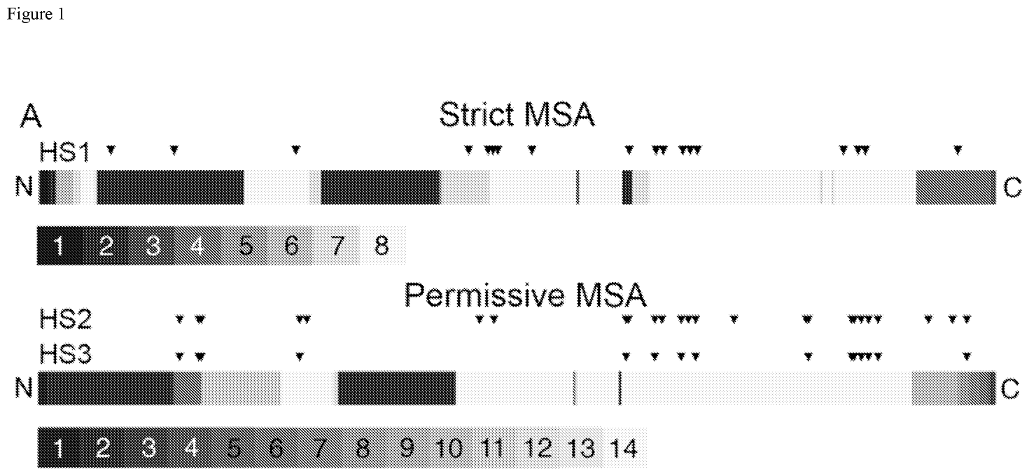

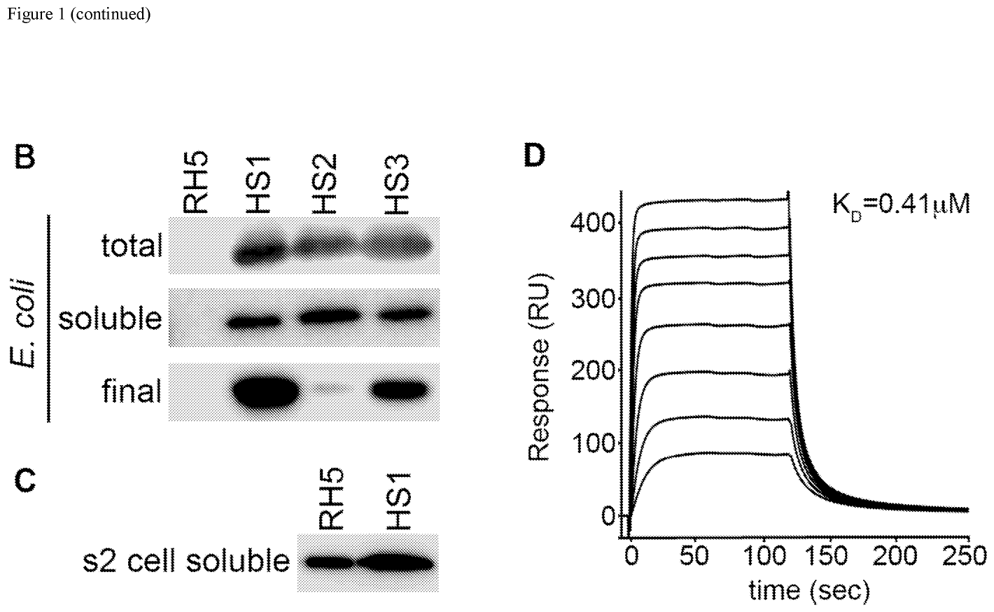

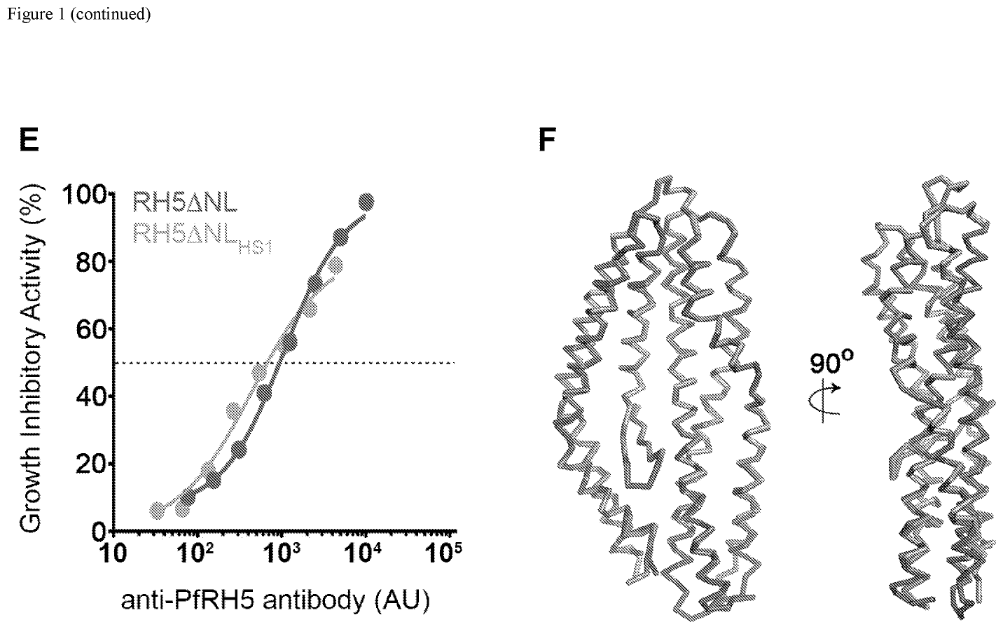

The designed modified PfRH5 antigens were then visually inspected, and three selected for experimental testing. One modified PfRH5 antigen, designated PfRH5ΔNLHS1, was based on the strict alignment bearing 18 mutations relative to the corresponding unmodified PfRH5 antigen (PfRH5ΔNL). The other two modified PfRH5 antigens were based on the permissive alignment bearing 25 and 15 mutations (designated PfRH5ΔNL_{HS2} and PfRH5ΔNL_{HS3}, respectively) (FIG. 1A Table 2).

### Example 2

### Expression and Purification of Thermally Stabilised Modified PfRh5 Antigen

Expression in E. coli

A gene for Plasmodium falciparum RH5 spanning from K141 to Q526 with both the flexible N-terminus (residues 1-140) and a disordered loop (residues 248-296) removed (PfRH5ΔNL) was available from a previous study (WO2016/016651). This gene had been codon optimized for expression in Drosophila melanagaster as C-terminal hexa-histidine tagged proteins (Invitrogen).

Synthetic genes of the modified PfRH5 antigens designed in Example 1 were made to match the boundaries of PfRH5ΔNL and were codon optimized for expression in D. melanogaster, giving the constructs PfRH5ΔNL_{HS1}, PfRH5ΔNL_{HS2}, and PfRH5ΔNL_{HS3}. PCR reactions were performed using the same primers for all the modified PfRH5 antigens and for PfRH5ΔNL and products were cloned into a modified pET15b vector (Novagen) encoding an N-terminal hexa-histidine tag and TEV protease cleavage site. This cloning strategy generated hexa-histidine tagged constructs pET15b-PfRH5ΔNL, pET15b-PfRH5ΔNL_{HS1}, pET15b-PfRH5ΔNL_{HS2} and pET15b-PfRH5ΔNL_{HS3}.

These constructs were introduced into the Rosetta-gami B(DE3)pLysS E. coli expression strain (Novagen). Bacterial cultures were grown overnight at 37° C. in 50 μg/ml ampicillin, 30 μg/ml kanamycin, 34 μg/ml chloramphenicol and 12.5 μg/ml tetracycline and inoculated in a 1:50 ratio into 1 L of fresh 2×YT medium supplemented with the same antibiotic concentrations. Cultures were grown at 37° C. until an OD₆₀₀ₙₘ, of 0.5-0.7 and gene expression was induced by addition of 0.5 mM IPTG for 4 hours at 37° C. Cultures were pelleted at 4,000 g for 10 minutes and resuspended in PBS buffer supplemented with EDTA-free protease inhibitors (Roche). Bacterial cell lysis was performed using a cell disruptor at a pressure of 30 Kpsi, followed by centrifugation at 20,000 g for 40 minutes. The soluble fraction of each construct was loaded into gravity flow column containing nickelnitrilotriacetic acid resin Ni²⁺-NTA, Qiagen) previously equilibrated with PBS buffer. The Ni²⁺-NTA resin was washed with 10 column volumes (CV) of PBS containing 30 mM imidazole, whilst the proteins were eluted in 3CV of PBS containing 0.7 M imidazole. The imidazole was removed by buffer exchange in PBS using a PD-1.0 desalting column (GE Healthcare) and protein analysis was performed by Western blot using a rabbit antibody antihexahistidine (Abeam) and a goat anti-rabbit HRP-conjugated antibody (Abeam). As the modified PfRH5 antigen PfRH5ΔNL_{HS1} showed the highest level of soluble protein expression, only this construct was scaled-up to expression in 6 L scale and purification as above. After Ni²⁺-NTA affinity purification, PfRH5ΔNL_{HS1} was further purified by size exclusion chromatography using a Superdex 200 16/60 column (GE Healthcare) previously equilibrated with 50 mM Tris-HCl pH=7.5, 200 mM NaCl. SEC-MALS analysis was performed using a S200 10/300 column (GE Healthcare) and data analysed using ORYX software (Oryx Systems).

### Expression in S2 Cells

The pExpress-2.1 vector containing the unmodified (wild type) PfRH5 for expression in Schneider 2 (S2) cells (pExpress-2, PfRH5ΔNL), was already available from a previous study (WO2016/016651). The PfRH5ΔNL_{HS1} gene was cloned into Express-2.1 vector using the Gibson cloning kit (NEB), giving the construct pExpress-2.1-PfRH5ΔNL_{HS1}. S2 cells were transfected with pExpress-2.1-PfRH5ΔNL_{HS1} as recommended by the supplier. Briefly, after thawing, S2 cells were grown to a density of 2.0×10⁶ cells/ml in Ex-cell medium (SAFC Biosciences) supplemented with 10% Fetal Calcium Serum (FCS) (Life Technologies) and 10 mg/ml penicillin-streptomycin (Sigma) medium. The cells were then spun at 300 g for 10 minutes and resuspended to a density of 2.0 million per ml in fresh Ex-cell medium with 10 mg/ml penicillin-streptomycin. Transfection was then performed adding 12.5 μg of DNA and 50 μL of EXPRES² TR 5× reagent Express²ion) to 2.5 ml of resuspended cells at 2.0 million cells/ml for 3 hours at 25° C. At the end of the incubation, FCS was added to the medium to a final concentration of 10% and cells were transferred to a Cellstar cell culture flask (Greiner) and grown in a static incubator at 25° C. At regular intervals of 4 days, half of the medium (2.5 ml) was removed and replaced with an equivalent amount of fresh Ex-cell medium containing 10 mg/ml penicillin-streptomycin and 10% FCS. In the third week, 10 ml of Excell medium supplemented with 2 mg/ml zeocin (Life Technologies) was added to the S2 cells upon transfer from Cellstar cell culture flask to a T25 flask. Cell growth was performed in a shaking incubator at 25° C. and cell were expanded every 4 days by dilution through the addition of Ex-cell medium to a final density of 8 million cells/ml. When the desired volume of S2 cells was reached, they were pelleted at 300 g for 10 minute and resuspended to a density of 8 million cells/ml in Ex-cell medium containing 10 mg/ml of penicillin-streptomycin (Sigma) before growth for 5 days at 25° C.

At the end of the 5 days of expression, the cells were harvested at 2000 g for 30 minutes and protein expression was assessed by Western blot analysis using the supernatant after centrifugation, which was normalized between PfRH5ΔNL and PfRH5ΔNL_{HS1} on the basis of the cell density at the time of the cell harvesting. Western blot was performed using a rabbit Anti-hexahistidine primary antibody (Abeam) and a goat anti-rabbit HRP-conjugated secondary antibody (Abcam). Western blot analysis was performed using a Li-cor system.

### Results and Discussion

Genes encoding each of the three modified PfRH5 antigens (PfRH5ΔNL_{HS1}, PfRH5ΔNL_{HS2} and PfRH5ΔNL_{HS3}) were designed and tested in the E. coli expression strain Rosetta Gami under different growth conditions as described above. Growth of the unmodified PfRH5 antigen PfRH5ΔNL was also examined as a control.

While no detectable soluble expression was observed for PfRH5ΔNL, each of the stabilized modified PfRH5 antigens expressed at similar levels (FIG. 1B). Protein from each modified PfRH5 antigen was purified by immobilised metal ion chromatography, followed by size exclusion chromatography. This showed PfRH5ΔNL_{HS1} to have the best production properties, yielding ˜4-fold more protein than PfRH5ΔNL_{HS3} and nearly 50-fold more than PfRH5ΔNL_{HS2}. PfRH5ΔNL_{HS1} was produced with a final yield of ˜1.3 mg from each litre of E. coli culture.

To determine if these improvements in expression properties are limited to prokaryotic expression systems, a stable Drosophila S2 cell line that expresses PfRH5ΔNL_{HS1} was also generated, allowing assessment of its expression levels in what is currently the leading system for (unmodified) RH5 antigen expression. Here too, an increased yield of protein was observed, with PfRH5ΔNL_{HS1} expressed at levels 3-4 fold higher than PfRH5ΔNL (FIG. 1C). Therefore the stabilized modified PfRH5 antigen expresses to significantly higher levels in Drosophila S2 cells and is the first version of PfRH5 to express in a stable, soluble, folded form in E. coli.

### Example 3

### Assessment of the Functionality of the Modified PfRH5 Antigens

### Expression and Purification of Basigin

Basigin was produced as previously described (WO2016/016651). In brief, residues 22-205 were expressed from a modified pEt15b in bacterial strain Origami B (DE3) (Novagen) by incubation overnight at 25° C. after induction with 1 mM IPTG. The protein was purified by Ni²⁺-NTA (Qiagen) affinity chromatography, followed by buffer exchange into PBS using a PD-10 desalting column (GE Healthcare), and overnight cleavage with His-tagged TEV protease at 4° C. before a second Ni²⁺-NTA column. The flow-through was concentrated using an Amicon Ultra centrifugal filter device (molecular mass cutoff, 3,000 Da). Finally, gel filtration was performed with a Superdex 200 16/60 column (GE Healthcare) in 20 mM HEPES (pH 7.5) and 150 mM NaCl.

### Expression and Purification of 9AD4 Monoclonal Antibody for Crystallisation

The hybridoma for the anti-PfRH5 monoclonal antibody 9AD4 was grown in Dulbecco's Modified Eagle's Medium (DMEM; Sigma) supplemented with 4 mM L-glutamine (Sigma), 0.01 M HEPES (Life Technologies), 100 U penicillin and 0.1 mg/ml streptomycin (Sigma), and 20% fetal calf serum (Gibco). It was then transferred into CD Hybridoma medium (Life Technologies) with glutamine, penicillin, and treptomycin. The cells were harvested after 7-10 days. The cell culture supernatant was exchanged into 20 mM phosphate pH 7.0 with a tangential flow filtration device (Pall).

The sample was then loaded onto a HiTrap Protein G HP column (GE Healthcare), eluted in 0.1 M glycine-HCl (pH 3.0), and immediately neutralised with 0.1 M Tris (pH 8.0). The sample was exchanged into 100 mM phosphate (pH 6.4), 300 mM NaCl, 2 mM EDTA, 5 mM L-cysteine (pH 6.4), and 1.5 mM β-mercaptoethanol using PD-10 columns (GE Healthcare).

Antibody Fab fragments were generated by addition of papain agarose (Sigma), and overnight incubation at 37° C. The papain agarose was removed by centrifugation, and the sample loaded onto a HiTrap Protein A HP column (GE Healthcare). Fab fragments did not bind to the column were gel filtered on a Superdex 200 16/60 column (GE Healthcare) in 20 mM HEPES (pH 7.5) and 150 mM NaCl.

### House Immunisations and Antibodies

6 week old BALB/c mice (Harlan, UK) were immunized intramuscularly with 2 doses of 20 μg protein in Addavax (Invivogen) two weeks apart. Blood samples were harvested by exsanguination two weeks after the second immunization. Polyclonal IgG was purified from pooled serum samples on Pierce Protein G agarose (ThermoFischer Scientific) and buffer exchanged using Amicon Ultra-15 Centrifugal Filter Units (Merck Millipore) into incomplete P. falciparum culture media. IgG concentration was determined using a Nanodrop 2000 (Thermos Scientific).

### In Vitro Assay of Growth Inhibitory Activity (GIA)

Long-term in vitro-cultured P. falciparum line 3D7, were grown in O Rh⁺ erythrocytes and 10% human serum. The standardized GIA assay from the NIH GIA Reference Center was used to assess the ability of antibodies to inhibit P. falciparum growth in vitro. Briefly, each test IgG was incubated with synchronized P. falciparum parasites for a single growth cycle, and relative parasitemia levels were quantified by biochemical determination of parasite lactate dehydrogenase. The Purified polyclonal IgG was tested in triplicate in a two-fold dilution series from 2 mg/ml down to 8 μg/ml.

### ELISA

A standardized ELISA was used to quantify the amount of PfRH5 specific antibodies in the purified IgG samples using previously described methodology. NUNC Maxisorp ELISA plates (Fisher) were coated overnight with 2 μg/ml full-length PfRH5 protein (3D7 sequence) with a C-terminal four amino acid C-tag, blocked with 5% skimmed milk (Marvel) in Dulbecco's PBS (Sigma). A standard curve was made using a reference serum sample, and for normalization each plate contained a characterized positive control. IgG was detected by HRP conjugated goat anti-mouse IgG diluted 1:1000 (Sigma, A3562) and developed using 1 mg/ml 4-nitrophenyl phosphate (Sigma) in diethanolamine buffer (Fisher) until the positive control reached an optical density (OD) 405 nm of 1.0 read by a ELx800 Microplate Reader (Bio-Tek) analysed using Gen5 ELISA software (Bio-Tek). Responses are reported in arbitrary units (AU) relative to the reference sample standard curve.

### Crystallisation, Data Collection and Data Processing of the PfRH5ΔNLHS1-9AD4 Complex

PfRH5ΔNL_{HS1} protein and 9AD4 antibody were expressed and purified as above. PfRH5ΔNL_{HS1} was loaded into a gravity flow column loaded with Ni²⁺-NTA resin first, followed by the addition of 9AD4 antibody in a 1:1 molar ratio. Bound PfRH5ΔNL_{HS1}-9AD4 complex was then washed with 10CV of 20 mM phosphate pH 7.5, 300 mM NaCl followed by an additional wash with 10CV of 20 mM phosphate pH 7.5, 1.50 mM NaCl (Buffer A). Bound complex was eluted with Buffer A supplemented with 0.5M imidazole and diluted 1:10 into Buffer A. Complex was cleaved overnight with endoproteinase GluC (Sigma) 1:100, GluC:V3 (w/w). Following cleavage, lysine methylation of the PfRH5ΔNL_{HS1}:9AD4 complex was performed as described above. The cleaved, methylated complex was injected into a Superdex 200 16/60 column (GE) previously equilibrated with 20 mM Hepes pH 7.5, 150 mM NaCl.

Purified PfRH5ΔNLHS1:9AD4 was concentrated to 6 mg/ml and crystallisation trials performed in 96 well plates by sitting drop vapour diffusion method. A screen was designed to optimize around the condition that had previously generated crystals of PfRH5ΔNL (screening 18-24% glycerol and 18-26% PEG1500). A TTP Labtech Mosquito LCP robot was used to dispense 100 mL of each protein complex at 6 mg/ml with 100 nl of well solution and 50 nl of Silver Bullet (Hampton Research). Plate-shaped crystals grew in 10 days at 4° C. with a well solution of 18% glycerol and 22% PEG 1500 and silver bullet additive 0.02M Hepes sodium pH 6.8 and benzidine, nicotinamide, pyromellitic acid and sulfaguanidine, each at 0.25% w/v.

Crystals were cryoprotected by addition of PEG400 directly into the crystallization drop to a final concentration of 30% and flash cooled in liquid N₂. Two datasets were collected from a single crystal at 100K at the Diamond Light Source synchrotron at beamline I04-1 (Didcot, Oxon, UK). For the first dataset, 900 images were collected at wavelength of 1.000 Å with an oscillation angle of 0.2° and exposure time of 0.1 s, whilst for the second dataset, another 900 images were collected from another portion of the crystal with the same oscillation range and same wavelength but exposure time of 0.3 s. The protein belonged to space group P2₁ with the unit cell parameters a=39.66 Å b=85.55 Å, c=132.87 Å; α=90.0°, β=90.91°, γ=90.0°. The two datasets were indexed using XDS (Acta Cryst. D, Biological Crystallography 66, 125-132 (2010)) and reduced together to 2.35 Å using AIMLESS (Acta Cryst. D, Biological Crystallography 62, 72-82 (2006)). The R_{free} set was generated randomly in UNIQUE (Acta Cry stallogr D Biol Crystallogr 67, 235-42 (2011)). The structure was solved by molecular replacement with PHASER (J Appl Crystallogr 40, 658-674 (2007)), using PfRH5ΔNL and 9AD4 as two separate searching models (Protein Database code 4U0R). Reiterated PHASER runs allowed the placement of one monomer of each protein in the asymmetric unit. The MR solution was refined in REFMAC (Acta Crystal. D Biol. Crystallogr. 60, 2184-2195 (2004)) by rigid body refinement (10.0-6.0 Å) and restrained refinement (42.77-2.35 Å) leading to initial R_{factor}=0.23 and R_{free}=0.27. Re-iterated model building was performed manually in COOT32. Jelly body refinement and TLS refinement were included in the final stages of the refinement in REFMAC yielding R_{factor}=0.17 and R_{free}=0.23. Input files defining the TLS groups for REFMAC were generated through the TLSMD server (Journal of Applied Crystallography 39, 109-111 (2006)). Structure validation was performed using MOLPROBITY (Acta Crystallogr D Biol Crystallogr 66, 12-21 (2010)), which confirmed that all the residues were located in the allowed regions of the Ramachandran plot. Crystallographic data and refinement statistics are reported in Table 3 below. Composite omit map was generated with PHENIX (Acta. Crystallogr D Biol Crystallogr 66, 213-21 (2010)).

TABLE 3
Data collection and refinement statistics
9AD4-PfRH5ΔNL_{HS1}complex

| | |
|---|---|
| Data collection | |
| Space group | P2₁ |
| Cell dimensions | |
| a, b, c (Å) | 39,66, 85.55, 132.87 |
| a, b, g (°) | 90.00, 90.91, 90.00 |
| Resolution (Å) | 42.77 (2.43-2.35) * |
| R_{sym} or R_{merge} | 0.096 (0.554) |
| I/sI | 13.9 (3.1) |
| Completeness (%) | 98.6 (97.9) |
| Redundancy | 6.5 (6.9) |
| Refinement | |
| Resolution (Å) | 42.77 (2.43-2.35 |
| No. reflections | 69953 (7137) |
| R_{work} | 0.173 (0.240) |
| R_{free} | 0.227 (0.326) |
| No. atoms | 6079 |
| Protein | 5747 |
| Ligand/ion | 2 |
| Water | 330 |
| B-factors | |
| Protein | 53.90 |
| Ligand/ion | 49.40 |
| Water | 51.10 |
| R.m.s. deviations | 0.008 |
| Bond lengths (Å) | 1.19 |
| Bond angles (°) | |

* Values in parentheses are for highest-resolution shell.

### Results and Discussion

Having demonstrated that the modified PfRH5 antigens of the invention express to high levels in Drosophila cells, and are also expressed in E. coli, the structural integrity and functionality of purified PfRH5ΔNL_{HS1} was then assessed.

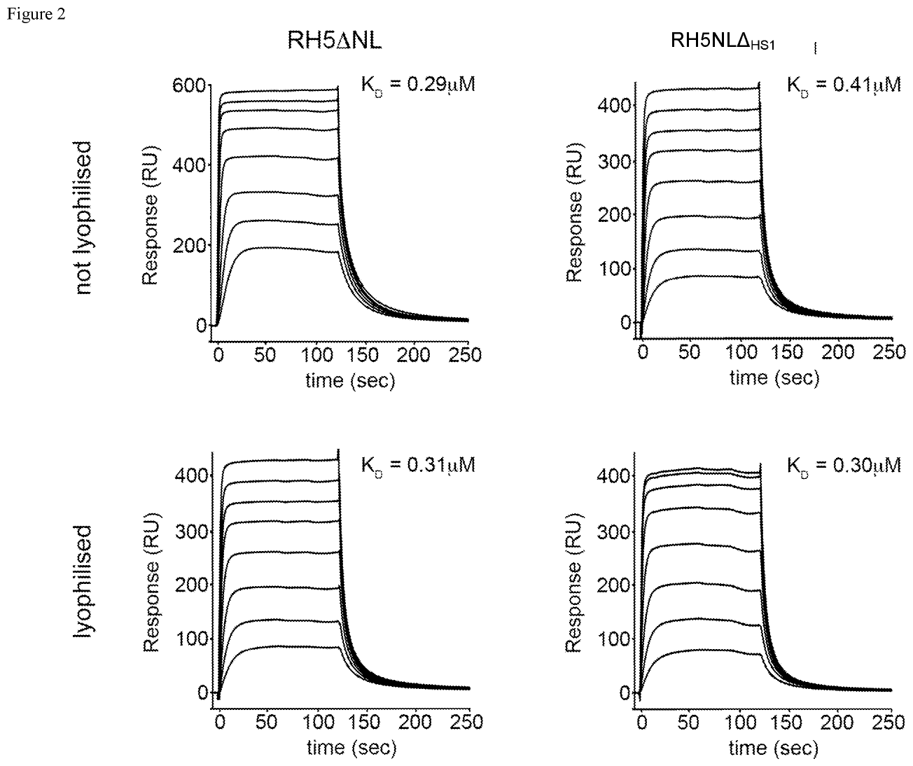

Surface plasmon resonance was used to show that PfRH5ΔNL_{HS1} bound to basigin with an affinity of 0.41 μM (FIG. 1D), comparable to the 0.29 μM observed for PfRH5ΔNL (FIG. 2). In addition, basigin binding was retained after desiccation and resuspension of both PfRH5ΔNL and PfRH5ΔNL_{HS1}, increasing the options for vaccine preparation (FIG. 2). To ensure that PfRH5ΔNL_{HS1} contains the epitopes required to elicit an inhibitory immune response, mouse polyclonal IgG were raised, and tested for their ability to neutralize parasites an in vitro assay of growth inhibition activity (GIA) (FIG. 1E). IgG raised against PfRH5ΔNL_{HS1} showed a strong inhibitory effect, similar to that for IgG raised against PfRH5ΔNL—indeed these polyclonal IgG were qualitatively comparable, requiring very similar amounts of PfRH5-specific IgG to neutralize 50% of parasites.

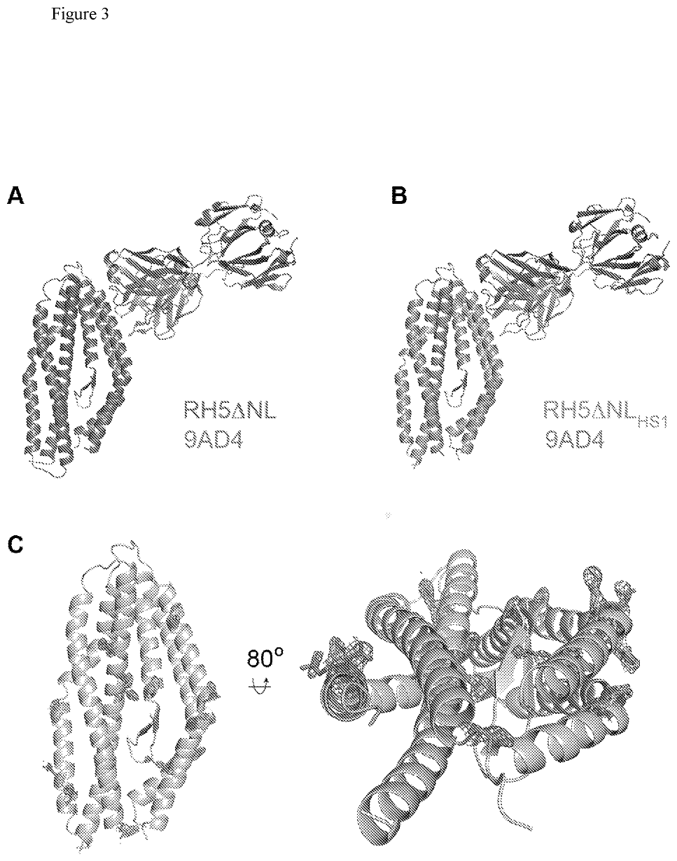

Finally, the crystal structure of PfRH5ΔNL_{HS1} in complex with the Fab fragment from an inhibitory monoclonal antibody, 9AD4, to 2.35 Å resolution was determined (FIG. 1F, FIG. 3, Table 3). Composite omit maps showed clear electron density for mutated residues, largely in their designed positions (FIG. 3). However, there were no other significant changes in the RH5 structure with root mean square deviations of 0.7 Å for the backbone Cα positions and 1.0 Å for the complex, when comparing PfRH5ΔNL_{HS1} and PfRH5ΔNL, each bound to 9AD4.

The stabilized and bacterially expressed modified PfRH5 antigen PfRH5ΔNL_{HS1} therefore retains the structure, ligand binding and immunogenic properties of unmodified (native) PfRH5.

### Example 4

### Assessment of the Thermal Stability of the Modified PfRH5 antigens

### Heat Treatment and Lyophilisation of PfRH5ΔNL and PfRH5ΔNL Variants

Purified protein samples of unmodified PfRH5ΔNL antigen and the modified PfRH5ΔNL_{HS1} antigen were normalized to a final 16 μM concentration and divided into 100 μL aliquots. Each aliquot was incubated for 1 hour in a PCR machine at the fixed temperature of 40, 45, 50, 52, 54, 56, 58, 60, 62, 64, 66, 68, 70 and 75° C. Each aliquot was flash cooled in liquid N₂ at the end of the heat treatment and all the aliquots were defrosted at the same time at room temperature before analysis by surface plasmon resonance (SPR). An additional aliquot of each protein sample was flash-cooled in liquid N₂ and lyophylized overnight. The samples were re-suspended in the same volume in deionized filtered water before SPR experiments.

### Surface Plasmon Resonance

Surface plasmon resonance (SPR) experiments were carried out using a BIAcore T200 instrument (GE Healthcare). Experiments were performed at 20° C. using a buffer containing 20 mM HEPES (pH 7.4), 150 mM NaCl, 3 mM EDTA, 0.005% Tween 20, 2 mg/ml dextran, and 1 mg/ml salmon sperm DNA (Sigma Aldrich). Basigin was immobilized on a CM5 chip (GE Healthcare) by amine coupling (GE Healthcare kit) to a total of 800 RU.

Lysine methylation of gel exclusion purified PfRH5ΔNL and PfRH5ΔNL_{HS1} was performed by incubation with 0.02M dimethylamine-borane complex (ABC) and 0.04M formaldehyde at 4° C. for 1 hour in the dark. After 1 hour an additional 0.02M ABC and 0.04M formaldehyde were added for a further hour. Finally, 0.01M ABC was added and the reaction was incubated at 4° C. overnight in the dark. The reaction mixture was quenched by addition of Tris-HCl to a final concentration of 50 mM, and the proteins were injected into a S200 16/60 column previously equilibrated with 20 mM Hepes 7.5, 150 mM NaCl. Gel filtered protein was concentrated to 16 μM using an Amicon 10 KDa cut-off concentrator (Millipore). A concentration series of PfRH5ΔNL or PfRH5ΔNL_{HS1} (8, 4, 2, 1, 0.5, 0.25, 0.125, and 0.0625 μM) was injected over the basigin-coated chip for 120 s at 30 μl/min, followed by a 300 s dissociation time. The chip surface was then regenerated with 30 s of 2 M NaCl. Specific binding was obtained by subtracting the response from a blank surface from that of the basigin-coated surface. The kinetic sensorgrams were fitted to a global 1:1 interaction model, allowing determination of the dissociation constant, Kd, using BIAevaluation software 1.0 (GE Healthcare). Data were plotted using the software GraphPad 7.0 (Prism).

### Circular Dichroism Analysis

The unmodified PfRH5ΔNL antigen and modified PfRH5ΔNL_{HS1} antigen were buffer exchanged into 20 mM phosphate 7.5, 150 mM NaF, using a PD-10 desalting column (Generon) and concentrated to 50 μg/ml. Circular Dichroism (CD) spectra were recorded between 200 nm and 250 nm with a temperature ramp increasing by increments of 2° C. from 20° C. to 90° C. Data analysis was performed using the software GraphPad 7.0 (Prism).

### Results and Discussion

To assess the thermal stability of PfRH5ΔNL_{HS1} circular dichroism with a thermal melt was conducted, following ellipticity at 220 nm as a measure of α-helicity (FIG. 2A). While ellipticity for the unmodified PfRH5ΔNL was reduced by 50% at 48° C., the stabilized modified PfH5 antigen, PfRH5ΔNL_{HS1}, showed the same loss of ellipticity at 66° C., showing a ˜20° C. improvement in thermal stability.

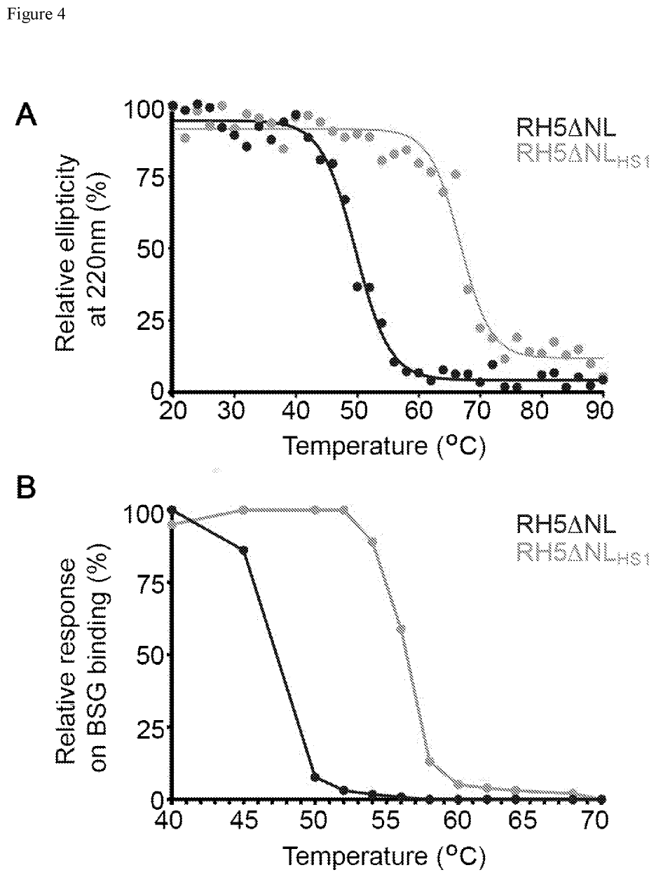

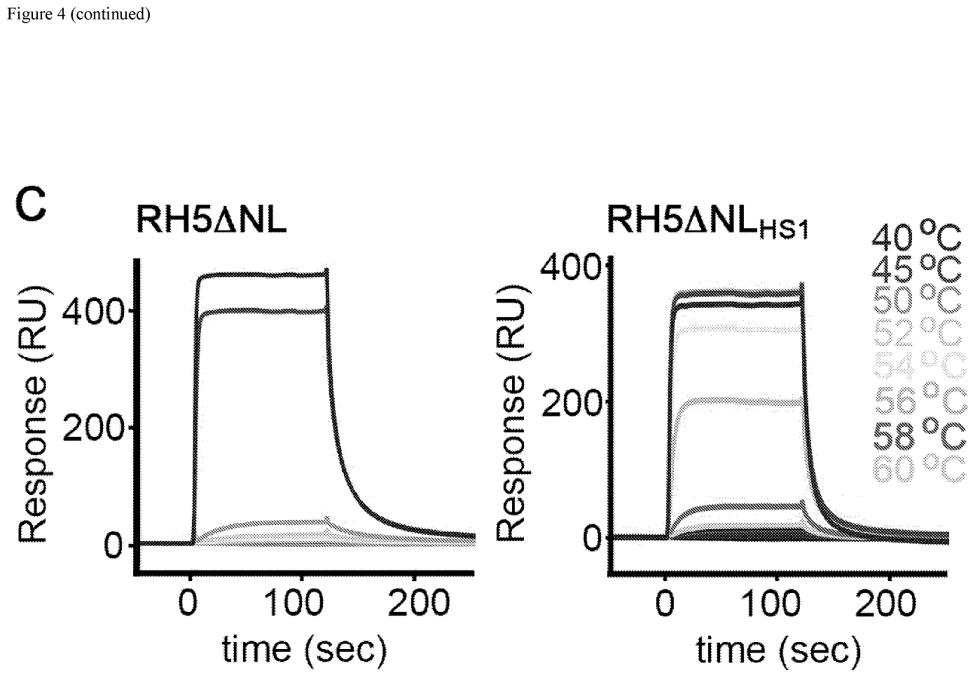

As circular dichroism measures secondary-structure content, rather than protein functionality, a further experiment was conducted in which unmodified PfRH5ΔNL and the stabilized modified PfH5 antigen, PfRH5ΔNL_{HS1}, were incubated at different temperatures for an hour before returning them to room temperature and testing basigin-binding by surface plasmon resonance (FIG. 4B, C). PfRH5ΔNL lost 50% of its basigin-binding capacity at ˜47° C. while PfRH5ΔNL_{HS1} showed a similar loss at ˜57° C., again showing a ˜10° C. improvement in thermal stability.

The modified PfRH5 antigens of the invention therefore retain both the structure and immunogenicity of the unmodified antigen, while allowing expression in E coli and increased thermal stability.

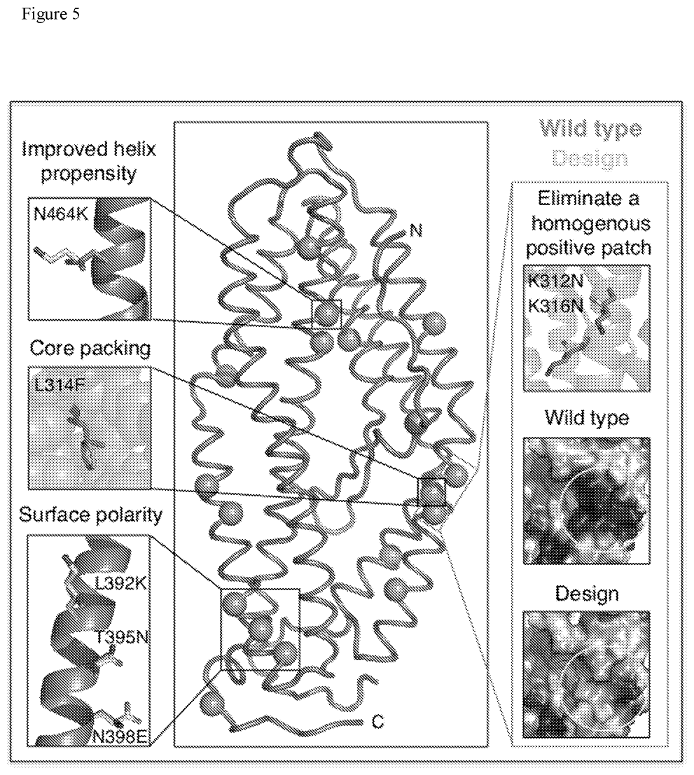

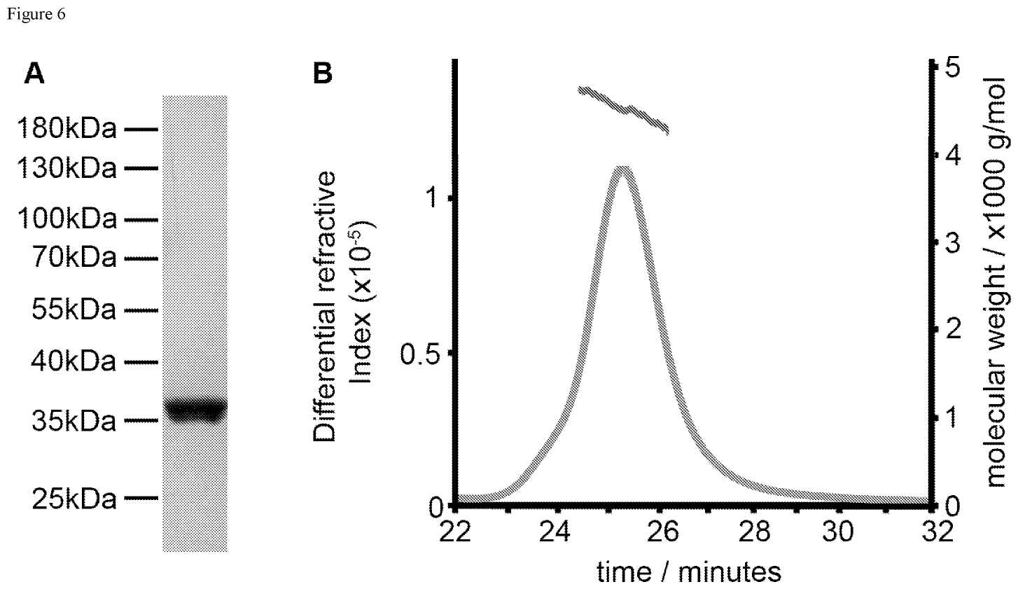

To assess the reasons for the higher bacterial expression level and improved thermal stability of PfRH5ΔNL_{HS1}, the structure of PfRH5ΔNL_{HS1} was compared with that of PfRH5ΔNL. The comparison showed several of the hallmarks expected for stabilizing mutations (FIG. 5, Table 4). In more detail, of the 18 mutations, 15 occurred on the surface, of which eight improved surface polarity, and an additional two (Lys312Asn and Lys316Asn) eliminated a homogenous positively charged patch. Indeed, such patches have previously been reported as associated with aggregation and poor stability.

Furthermore, RH5 comprises a mostly helical backbone (75% of the sequence), and seven mutations increased helix-forming propensity in the modified PfRH5 antigen PfRH5ΔNL_{HS1} relative to unmodified PfRH5.

Lastly, PfRH5ΔNL_{HS1} contains three mutations that improve hydrophobic core packing (Asp183Glu, Leu314Phe and Ser286Ala).

Thus, by combining a large set of mutations in a single variant, significant gains in expression levels and thermal stability can be achieved.

TABLE 4
structure features compared to PfRH5ΔNL

| Structural Feature | Contributing mutations |
|---|---|
| Mutations at buried positions^{a} | D183E, L314F, S286A |
| Mutations improving helical | I157L, D183E, S370A, T384K, N398E, |
| propensity^{a} | N464K, S467A |
| Surface mutations increasing | A233K, M330N, S381A, T384K, L392K, |
| polarity^{a} | T395N, N398E, N464K |
| Mutations in homogenous positive | K312N, K316N |
| patch^{a} | |

^{a}Numbering according to Protein Database entry 4WAT.

### Conclusions

In summary, the present inventors have used a computational method known as PROSS to design improved expression properties and thermal stability into the leading malaria vaccine candidate, PfRH5. The output of the PROSS has been used to generate an improved immunogen, i.e. a modified PfRH5 antigen that is more economical and scalable for production and more thermally stable for storage and delivery. This immunogen will be valuable in future generations of malaria vaccines.

The approach used here is also highly applicable for the development of vaccine immunogens against other stages of the life cycle of P. falciparum or other pathogens. While this will be an issue for some other vaccine candidates, the majority of pathogen surface proteins, from viral surface proteins to the diverse PfEMP1 family of Plasmodium surface proteins, are highly variable as they evolve under diversifying selection pressure to avoid detection by the acquired immune system. With judicious selection of natural variants on which to base the design process, the methods outlined here will be applicable to many of these cases, whether highly conserved or highly diverse. This method is therefore expected to contribute to vaccine immunogen production across a wide range of the most deadly diseases affecting humanity.

### Example 5

### In Vivo Use of Modified PfRH5 Antigen Vaccines in Primates

### Vaccine

The vaccines used are unadjuvanted replication-deficient viral vectors for PfRH5ΔNL_{HS1}, with adenoviruses used for priming (likely serotype AdHu5, ChAd63, ChAdOX1 or ChAdOX2), and poxviruses used for boosting (Modified Vaccinia Ankara, MVA).

Viral vector vaccines are stored at −80° C. or on dry ice prior to use, then thawed and are stable at 4° C./on ice for at least 2 hours. Vaccines are prepared for administration by dilution in PBS, which can be performed at an earlier date (followed by re-freezing) if necessary.

Doses to be used are calculated with reference to tolerability of vectored vaccines in humans, and doses used in previous rabbit studies with this antigen. The vaccines express fragments of the blood-stage P. falciparum antigen RH5. It has previously been demonstrated that vaccines expressing full length RH5 are immunogenic in mice, rabbits, Aotus monkeys and humans (see, Douglas et al. Cell Host Microbe (2015), 17(1):130-139, which is herein incorporated by reference in its entirety). The antibodies induced are highly effective in GIA.

### Preparation of Animals

Aotus nancymaae can be sourced from San Marcos University captive breeding programme and housed in AAALAC-accredited facilities at NAMRU-6.

Animals can be used which have previously been used in other studies, provided they are malaria-naive and have intact spleens. Possible confounding differences between animals (e.g. age, weight, type of previous use) could be addressed by stratified randomisation of animals to study groups.

Provisional group structure is as follows:

| | Group number | Vaccine antigen | Number |
|---|---|---|---|
| VACCINE | 1 | AMA1 +/− MSP1 | 8 |
| STUDY | 2 | PfRH5FL viral vectors | 8 |
| | 3 | RH5ΔNL viral vectors | 8 |
| | 4 | PfRH5ΔNL_{HS1} viral | 8 |
| | | vectors | |
| | 5 | Empty vectors (no | 8 |
| | | malaria antigen; negative | |
| | | control) | |
| THERAPY | 6 | PfRH5FL monoclonal | 5 |
| STUDY | | antibody | |
| | 7 | RH5ΔNL monoclonal | 5 |
| | | antibody | |
| | 8 | PfRH5ΔNL_{HS1} | 5 |
| | | monoclonal antibody | |
| | 9 | RH5ΔNL aptamer | 5 |
| | 10 | PfRH5ΔNL_{HS1} aptamer | 5 |
| | 11 | Untreated infection | 5 |
| | | controls | |
| | N/A | N/A-challenge donor | 1 |
| | | Total | 71 |

Precise size and number of groups is determined with statistical advice, after review of the variability in outcomes in non-vaccinated control Aotus in previous P. falciparum challenge trials.

### Administration of Vaccine

An 8 week prime-boost interval gives reliable immunogenicity in mouse, rabbit, macaque and humans with these and related vectors.

P. falciparum Challenge

Challenge is performed 2 weeks post-boost, at which time antibody responses were at or near maximum in a macaque study of related vectors (S. J. Draper et al., J. Immunol. (2010)) and in a related Aotus monkey study (see, Douglas et al. Cell Host Microbe (2015), 17(1): 130-139).

Frozen vials of FVO parasites are available at NAMRU-6. Optimal dose of parasites to be used for challenge is determined by balancing improved reliability of outcome in negative control animals if higher dose used, versus possible improved sensitivity of efficacy detection with prolonged period of parasitaemia if a lower dose is used.

10,000 ring-stage parasites appear to be commonly used, obtained by dilution of blood of a donor monkey with microscopically-patent parasitaemia (S. Dutta et al., Plus One 4, (2009)).

The schedulers as follows:
Day −1: Pre-immune bleed (c. 2 ml blood, for serum+/−PBMCs). This can be performed immediately prior to vaccination on day 0 if preferable for convenience of animal handling.
Day 0: Prime vaccination (adenovirus vectors in PBS, c. 200 ul intramuscular)
Day 14: Post-prime immuno-monitoring bleed (c. 2 ml blood, for serum+/−PBMCs)
Day 49: Optional immuno-monitoring bleed (0.5 ml blood, for serum)
Day 55: Pre-boost immuno-monitoring bleed (c. 2 ml blood, for serum+/−PBMCs). This can be performed immediately prior to boost vaccination on day 56 if preferable for convenience or animal handling.
Day 56: Boost vaccination (MVA vectors in PBS, 200-400 ul intramuscular)
Day 69=Day C-1: Post-boost immuno-monitoring bleed (c. 2 ml blood, for serum+/−PBMCs). This can be performed immediately prior to challenge on day 70 if preferable for convenience or animal handling.
Day 70=Day C+0: Challenge with FVO parasites. Dose and protocol TBC as above.
Daily from day C+3 until treatment endpoint: Clinical symptom scoring. Bleeds for parasitaemia monitoring by microscopy+/−QPCR; measurement of hematocrit and/or hemoglobin.concentration. See below for treatment endpoints.
Day of treatment: Post-challenge immuno-monitoring bleed (0.5 ml blood, for serum)
˜Day 91=Day C+21: End of challenge phase of study.
Re-challenge: a second challenge of the animals is envisaged.

### Immunological and Parasitological Assays

Antigen-specific antibody titers are quantified by ELISA at multiple timepoints.

### Additional Assays Include:

GIA (pre-challenge timepoint; 70% GIA at 1:10 serum dilution has been proposed as a correlate of vaccine-induced protection in Aotus);

IFA (pre-challenge timepoint);

ADRB;

QPCR monitoring of parasite density

ELISPOT or ICS quantification of antigen-specific T cells.

### Endpoints

Different possible endpoints have been proposed for Aotus-P. falciparum challenges. Cumulative parasitemia calculated by summing daily parasitemia from the day of challenge until the day the first animal in the study is treated for any reason has been used in some recent studies and, by virtue of being a continuous variable, may have statistical advantages (J. A. Lyon et al., Plos One 3, (2008)).

Humane drug treatment endpoints are employed which may include the following:

clinical symptoms exceeding a pre-defined score,

a threshold level of uncontrolled parasitaemia e.g. 200,000p/μl or 5%,

a threshold level of anaemia,

reaching a pre-specified day post-challenge, e.g. C+21.

### Example 6

### Use of Modified PfRH5 Antigen Vaccine in Humans

A clinical trial has already been conducted for unmodified full-length PfRH5 (NCT02181088, https://clinicaltrials.gov/ct2/show/NCT02181088?term=vac057&rank=1). A similar strategy is used in connection with the modified PfRH5 antigens of the invention, as set out below.

### Construction of Vaccine

Viral-vector expressed modified PfRH5 antigen (PfRH5ΔNL_{HS1}) is generated from MVA, or AdHu5 or ChAd63 or ChAdOX1 or ChAdOX2. The process is initiated using a plaque-purified recombinant and GMP-certified HEK293 cells (available at the Jenner Institute Clinical Biomanufacturing Facility). A single batch of >1.2×10¹³ viral particles (vp) is generated. Release assays are according to the European Pharmacopoeia. Absence of replication competent virus is demonstrated. The MVA-PfRH5ΔNL_{HS1} antigen is used as a boosting agent and is manufactured in chicken embryo fibroblasts (CEFs). The seed stock virus is supplied for production of the master seed virus/working seed virus (MSV/WSV). A clinical lot is then produced from the WSV. Vaccine toxicology studies are undertaken

### Administration of Vaccine

Volunteers receive various dose schedules of viral-vector expressed PfRH5ΔNL_{HS1} in groups. The sample size is sufficient to monitor routine and/or unexpected local and systemic AEs, whilst providing a thorough analysis of vaccine-induced cellular and humoral immunogenicity. Vaccine safety and immunogenicity is monitored in detail and analysed between dosing/regime groups using appropriate non-parametric statistics for small group sizes.

Vaccine-induced antigen-specific IgG function is assessed by in vitro assays of growth inhibitory activity (GIA) against P. falciparum strain 3D7, FVO, 7G8 and/or Dd2 parasites.

All vaccinations are administered intramuscularly in the deltoid muscle of the upper arm. This route of administration has been shown to be safe for other ChAd63 vaccines and to significantly reduce local AEs in comparison to intradermal vaccination.

Volunteers in Group 1 receive a dose of 5×10⁹ vp of ChAd63 PfRH5ΔNL_{HS1} (Group 1) and volunteers in Group 2 receive the full dose of 5×10¹⁰ vp of ChAd63 PfRH5ΔNL_{HS1} (Group 2). This two-step dose escalation for the ChAd63 vaccine vector has been applied to ChAd63-PfMSP1, ChAd63-PfAMA1 and ChAd63-PvDBP in clinical trials without any safety issues arising.

Within Group 2 (5×10¹⁰ vp ChAd63-PfRH5ΔNL_{HS1}), two sub-groups of volunteers (2B and 2C) are boosted after 8 weeks with an escalating dose of MVA-PfRH5ΔNL_{HS1}. Group 2A represents non-boosted controls.

The doses of MVA-PfRH5ΔNL_{HS1} are 1×10⁸ pfu for Group 2B, and 2×10⁸ pfu for Group 2C. A dose of 1-2×10⁸ pfu is the standard dose currently used in other studies of MVA vaccines encoding ME-TRAP, PfMSP1, PfAMA1, PfCSP or PvDBP.

Assessment following Administration of Antigen

Safety and tolerability of viral-vector expressed PfRH5ΔNL_{HS1} is assessed by comparing the frequency and severity of both local and systemic adverse events (AEs) between the dosing groups, including using diary cards for the first week. Details of AEs are collected at each clinic visit, along with a medical examination. Blood samples for haematology and biochemistry are taken at screening, and days 14, 28, 56, 63, 84 and 140.

Humoral and cellular immunogenicity of viral-vector expressed PfRH5ΔNL_{HS1} vaccines administered in the various dosing regimens is assessed. Immunological blood samples are taken at screening and days 0, 1, 4, 7, 14, 28, 56, 57, 60, 63, 84, 112 and 140 with respect to ChAd63-PfRH5ΔNL_{HS1} vaccination on day 0 and MVA-PfRH5ΔNL_{HS1} vaccination on day 56.

PfRH5ΔNL_{HS1}-specific immunogenicity is assessed by a variety of immunological assays including total IgG, isotype and avidity ELISA, GIA, memory B cell and plasma cell (ASC) ELIspot, ex-vivo IFN-γ ELISPOT, multiparameter flow cytometry and more exploratory assays including host gene expression studies post-vaccination.

### Sporozoite Challenge

Once adequate immunogenicity is observed—defined as >20% GIA activity in at least half the vaccinees—a further group of subjects is vaccinated with the most immunogenic regime identified.

These subjects are challenged (along with non-vaccinated controls) with a number (e.g. 5) of infectious mosquito bites. This procedure is now well established by the Imperial College (R Sinden)—Oxford—Walter Reed (J Murphy) team and over 250 individuals have been challenged in the last six years.

Control volunteers develop patent parasitaemia at, on average, 11 days post challenge and those who do not develop malaria by day 21 are considered fully protected. The subjects are monitored carefully for any evidence of immunopathology (although this is very unlikely at the low parasite densities that are reached prior to treatment).

A real-time PCR assay to quantify blood-stage infection is used twice a day during the key follow-up period from day 6.5 to 14.0 post challenge (and daily thereafter). This has proved valuable in monitoring rates of parasite growth in vaccinees, recently providing evidence of measurable but low level blood-stage efficacy with the PEV3a vaccine.

### Assessment Following Sporozoite Challenge

As in the above assessment following administration of antigen, detailed immunomonitoring is undertaken and, in this case, correlates of GIA activity and/or immune responses with efficacy are searched for.

Fully protected volunteers are invited to undergo a re-challenge at six months after their final vaccination to determine the durability of protection.

### Example 7

### In Vivo Treatment of Malaria in Primates using PfRH5ΔNL_{HS1} Binding Agents

### Construction of Binding Agents

Construction of Bind PfRH5ΔNL_{HS1}-Binding Monoclonal Antibodies.

Murine monoclonal antibodies which specifically bind PfRH5ΔNL_{HS1} in an ELISA are isolated from hybridomas generated by fusing splenocytes from mice immunized with PfRH5ΔNL_{HS1} with myeloma cells. It is confirmed that these antibodies recognise native parasites in an indirect immunofluorescence assay, and inhibit parasite growth in GIA.

A panel of mAbs is generated which are capable of binding PfRH5ΔNL_{HS1} by ELISA. BALB/c mice are immunised with adenovirus and MVA-vectored PfRH5ΔNL_{HS1} vaccines at doses of 1×10⁸ infectious units and 1×10⁷ plaque forming units respectively, and with an 8 to 12 week prime-boost interval. Splenocytes are fused with Sp2 myeloma cells, according to previously published methods (Yokoyama, W. M et al. Curr Protoc Immunol (2006)). Hybridoma supernatants are screened for binding to recombinant PfRH5ΔNL_{HS1} protein by ELISA, using previously published methods.

The ability of each of the mAbs to neutralize 3D7-strain parasites is tested in a GIA assay.

Previously published methods are used to minimize the immunogenicity of the monoclonal antibody in order to make it suitable for human use, such as replacement of the murine Fc region with a human Fc region of a chosen Ig class and subtype.

### Construction of PfRH5ΔNL_{HS1}-Binding Polyclonal Antibodies

Rabbits are immunised with PfRH5ΔNL_{HS1}. Rabbit immunisations are carried out by Biogenes (Germany), Female ZiKa rabbits (n=4) are immunised intramuscularly (i.m.) with 20 μg protein on day 0, formulated in complete Freund's adjuvant, followed by two booster immunisations i.m. on days 28 and 56 with the same dose of protein formulated in incomplete Freund's adjuvant. Control rabbits receive the same immunisation schedule with 50 μg ovalbumin protein. Serum is collected two weeks after the final immunisation and shipped frozen.

Total IgG is purified from rabbit sera using protein G columns (Pierce). The P. falciparum 3D7 and 7G8 lines are maintained in continuous culture using fresh O⁺ erythrocytes at 2% haematocrit and synchronised by two incubations in 5% sorbitol 6-8 h apart. Synchronised trophozoites are adjusted to 0.3% parasitaemia and then incubated for 42 h with the various IgG concentrations (tested in triplicate) Final parasitaemia is determined by biochemical determination of parasite lactate dehydrogenase. Percentage growth inhibition is expressed relative to wells containing IgG from control immunised rabbits. The mean of the three replicate wells is taken to obtain the final data for each individual rabbit at each tested IgG concentration. Experiments are performed twice against each strain of parasite with very similar results.

The assay of GIA is performed using the method of the MVI/NIH, reference laboratory (as set out in K. Miura et al., Clinical and Vaccine Immunology 16, 963 (2009)). Total IgG was purified using Protein G (Pierce).

Results are calculated relative to growth in the presence of 10 mg/mL IgG from a rabbit immunized with non-malaria control vaccines.

It is found that IgG induced by PfRH5ΔNL_{HS1} potently inhibited parasite growth of both the 7G8 and 3D7 parasite strains.

EC₅₀ values are estimated for the GIA effect of anti-PfRH5ΔNL_{HS1} IgG against the 3D7 and 7G8 parasite strains. The results show that the PfRH5ΔNL_{HS1} vaccine induces the levels of functional GIA against both homologous (7G8) and heterologous (3D7) parasites, comparable to the GIA observed using a vaccine of the corresponding unmodified PfRH5 antigen, PfRH5 ΔNL.

### Construction of PfRH5 ΔNL_{HS1}-Binding Aptamers

An oligonucleotide aptamer which specifically bind PfRH5ΔNL_{HS1} is identified using known methods (as set out e.g. in D. H. J. Bunka, P. G. Stockley, Nature Reviews Microbiology 4, 588 (2006)). It is confirmed that this molecule recognizes native parasites in an indirect immunofluorescence assay, and inhibited parasite growth in GIA.

Previously published methods are used to optimize the pharmacokinetics (half-life and biodistribution) of the aptamer, to render it suitable for therapeutic use.

The aptamer is conjugated to a monoclonal antibody to modify its pharmacokinetics and/or recruit Fe-dependent immune functions.

### Preparation of Animals

This is carried out as in Example 5 above.

P. falciparum Challenge

This is carried out as in Example 5 above, with the exception that malaria- and vaccine-naïve monkeys are infected with P. falciparum FVO parasites.

### Treatment

At a pre-determined point at which all monkeys exhibit microscopically quantifiable parasitaemia, the therapeutic agents (monoclonal antibody or aptamer) are administered at high dose.

The dosage regime in the case of monoclonal antibody is in the region of 1 mg/ml blood volume. The dosage regime in the case of aptamers is the molar equivalent (around 7 μM)).

The outcome of infection is compared to infected but untreated control monkeys.

### Immunological and Parasitological Assays

This is carried out as in Example 5 above.

### Endpoints

These are considered as in Example 5 above.

### Sequence Information

### Exemplary Unmodified PfRH5 Antigens

Full length PfRH5 amino acid sequence (3D7) including signal sequence: SEQ ID NO: 1

| | |
|---|---|
| 1 | [symbol] [symbol] [symbol] SFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FLDYYKHLSY NSIYHKSSTY GKCIAVDAFI KKINETYDKV KSKCNDIKND LIATIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLILS VKSKNLNKDL SDMTNILQQS ELLLTNLNKK MGSYIYIDTI KFIHKEMKHI |
| 421 | FNRIEYHTKI INDKTKIIQD KIKLNIWRTF QKDELLKRIL DMSNEYSLFI TSDHLRQMLY |
| 481 | NTFYSKEKHL NNIFEHLIYV LQMKFNDVPI KMEYFQTYKK NKPLTQ |

Signal sequence (amino acids 1 to 23) is in bold italics, flexible N-terminal (amino acids 1 to 139) and flexible loop (amino acids 248 to 296) regions are underlined.

Full length PfRH5 amino acid sequence (7G8) including signal sequence: SEQ ID NO: 2

| | |
|---|---|
| 1 | [symbol] [symbol] [symbol] SFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FLDYYKHLSY NSIYHKSSTY GKYIAVDAFI KKINETYDKV KSKCNDIKND LIATIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLILS VKSKNLNKDL SDMTNILQQS ELLLTNLNKK MGSYIYIDTI KFIHKEMKHI |
| 421 | FNRIEYHTKI INDKTKIIQD KIKLNIWRTF QKDELLKRIL DMSNEYSLFI TSDHLRQMIY |
| 481 | NTFYSKEKHL NNIFHHLIYV LQMKFNDVPI KMEYFQTYKK NKPLTQ |

Signal sequence (amino acids 1 to 23) is in bold italics, flexible N-terminal (amino acids 1 to 139) and flexible loop (amino acids 248 to 296) regions are underlined.

PfRH5 amino acid sequence (3D7) excluding signal sequence and flexible N-terminal region (amino acids 1 to 139): SEQ ID NO: 3

| | |
|---|---|
| 1 | KNVNFLQYHF KELSNYNIAN SIDILQEKEG HLDFVIIPHY TFLDYYKHLS YNSIYHKSST |
| 61 | YGKCIAVDAF IKKINEAYDK VKSKCNDIKN DLIATIKKLE HPYDINNKND DSYRYDISEE |
| 121 | IDDKSEETDD ETEEVEDSIQ DTDSNHAPSN KKKNDLMNRA FKKMMDEYNT KKKKLIKCIK |
| 161 | NHENDFNKIC MDMKNYGTNL FEQLSCYNNN FCNTNGIRYH YDEYIHKLIL SVKSKNLNKD |
| 241 | LSDMTNILQQ SELLLTNLNK KMGSYIYIDT IKFIHKEMKH IFNRIEYHTK IINDKTKIIQ |
| 301 | DKIKLNIWRT FQKDELLKRI LDMSNEYSLF ITSDHLRQML YNTFYSKEKH LNNIFHHLIY |
| 361 | VLQMKFNDVP IKMEYFQTYK KNKPLTQ |

Flexible loop (corresponding to amino acids 248 to 296 of full length PfRH5 of SEQ ID NO: 1) region is underlined.

PfRH5 amino acid sequence (7G8) excluding signal sequence and flexible N-terminal region (amino acids 1 to 139): SEQ ID NO: 4

| | |
|---|---|
| 1 | KNVNFLQYHF KELSNYNIAN SIDILQEKEG HLDFVIIPHY TFLDYYKHLS YNSIYHKSST |
| 61 | YGKYIAVDAF IKKINEAYDK VKSKCNDIKN DLIATIKKLE HPYDINNKND DSYRYDISEE |
| 121 | IDDKSEETDD ETEEVEDSIQ DTDSNHAPSN KKKNDLMNRA FKKMMDEYNT KKKKLIKCIK |
| 181 | NHENDFNKIC MDMKNYGTNL FEQLSCYNNN FCNTNGIRYH YDEYIHKLIL SVKSKNLNKD |
| 241 | LSDMTNILQQ SELLLTNLNK KMGSYIYIDT IKFIHKEMKH IFNRIEYHTK IINDKTKIIQ |
| 301 | DKIKLNIWRT FQKDELLKRI LDMSNEYSLF ITSDHLRQML YNTFYSKEKH LNNIFHHLIY |
| 361 | VLQMKFNDVP IKMEYFQTYK KNKPLTQ |

Flexible loop (corresponding to amino acids 248 to 296 of full length PfRH5 of SEQ ID NO: 2) region is underlined.

PfRH5 amino acid sequence (3D7) excluding signal sequence and flexible N-terminal region (amino acids 1 to 159): SEQ ID NO: 5

| | |
|---|---|
| 1 | SIDILQEKEG HLDFVIIPHY TFLDYYKHLS YNSIYHKSST YGKCIAVDAF IKKINEAYDK |
| 61 | VKSKCNDIKN DLIATIKKLE HPYDINNKND DSYRYDISEE IDDKSEETDD ETEEVEDSIQ |
| 121 | DTDSNHAPSN KKKNDLMNRA FKKMMDEYNT KKKKLIKCIK NHENDFNKIC MDMKNYGTNL |
| 181 | FEQLSCYNNN FCNTNGIRYH YDEYIHKLIL SVKSKNLNKD LSDMTNILQQ SELLLTNLNK |
| 241 | KMGSYIYIDT IKFIHKEMKH IFNRIEYHTK IINDKTKIIQ DKIKLNIWRT FQKDELLKRI |
| 301 | LDMSNEYSLF ITSDHLRQML YNTFYSKEKH LNNIFHHLIY VLQMKFNDVP IKMEYFQTYK |
| 361 | KNKPLTQ |

Flexible loop (corresponding to amino acids 248 to 296 of full length PfRH5 of SEQ ID NO: 1) region is underlined.

PfRH5 amino acid sequence (7G8) excluding signal sequence and flexible N-terminal region (amino acids 1 to 159): SEQ ID NO: 6

| | |
|---|---|
| 1 | SIDILQEKEG HLDFVIIPHY TFLDYYKHLS YNSIYHKSST YGKYIAVDAF IKKINEAYDK |
| 61 | VKSKCNDIKN DLIATIKKLE HPYDINNKND DSYRYDISEE IDDKSEETDD ETEEVEDSIQ |
| 121 | DTDSNHAPSN KKKNDLMNRA FKKMMDEYNT KKKKLIKCIK NHENDFNKIC MDMKNYGTNL |
| 181 | FEQLSCYNNN FCNTNGIRYH YDEYIHKLIL SVKSKNLNKD LSDMTNILQQ SELLLTNLNK |
| 241 | KMGSYIYIDT IKFIHKEMKH IFNRIEYHTK IINDKTKIIQ DKIKLNIWRT FQKDELLKRI |
| 301 | LDMSNEYSLF ITSDHLRQML YNTFYSKEKH LNNIFHHLIY VLQMKFNDVP IKMEYFQTYK |
| 361 | KNKPLTQ |

Flexible loop (corresponding to amino acids 248 to 296 of full length PfRH5 of SEQ ID NO: 2) region is underlined.

PfRH5 amino acid sequence (3D7) excluding signal sequence, flexible N-terminal (amino acids 1 to 139) and flexible loop (amino acids 248 to 296) regions: SEQ ID NO: 7

| | |
|---|---|
| 1 | KNVNFLQYHF KELSNYNIAN SIDILQEKEG HLDFVIIPHY TFLDYYKHLS YNSIYHKSST |
| 61 | YGKCIAVDAF IKKINEAYDK VKSKCNDIKN DLIATIKKLE HPYDINNKNR AFKKMMDEYN |
| 121 | TKKKKLIKCI KNHENDFNKI CMDMKNYGTN LFEQLSCYNN NFCNTNGIRY HYDEYIHKLI |
| 181 | LSVKSKNLNK DLSDMTNILQ QSELLLTNLN KKMGSYIYID TIKFIHKEMK HIFNRIEYHT |
| 241 | KIINDKTKII QDKIKLNIWR TFQKDELLKR ILDMSNEYSL FITSDHLRQM LYNTFYSKEK |
| 301 | HLNNIFHHLI YVLQMKFNDV PIKMEYFQTY KKNKPLTQ |

PfRH5 amino acid sequence (7G8) excluding signal sequence, flexible N-terminal (amino acids 1 to 139) and flexible loop (amino acids 248 to 296) regions: SEQ ID NO: 8

| | |
|---|---|
| 1 | KNVNFLQYHF KELSNYNIAN SIDILQEKEG HLDFVIIPHY TFLDYYKHLS YNSIYHKSST |
| 61 | YGKYIAVDAF IKKINEAYDK VKSKCNDIKN DLIATIKKLE HPYDINNKNR AFKKMMDEYN |
| 121 | TKKKKLIKCI KNHENDFNKI CMDMKNYGTN LFEQLSCYNN NFCNTNGIRY HYDEYIHKLI |
| 181 | LSVKSKNLNK DLSDMTNILQ QSELLLTNLN KKMGSYIYID TIKFIHKEMK HIFNRIEYHT |
| 241 | KIINDKTKII QDKIKLNIWR TFQKDELLKR ILDMSNEYSL FITSDHLRQM LYNTFYSKEK |
| 301 | HLNNIFHHLI YVLQMKFNDV PIKMEYFQTY KKNKPLTQ |

PfRH5 amino acid sequence (3D7) excluding signal sequence, flexible N-terminal (amino acids 1 to 159) and flexible loop (amino acids 248 to 296) regions: SEQ ID NO: 9

| | |
|---|---|
| 1 | SIDILQEKEG HLDFVIIPHY TFLDYYKHLS YNSIYHKSST YGKCIAVDAF IKKINEAYDK |
| 61 | VKSKCNDIKN DLIATIKKLE HPYDINNKNR AFKKMMDEYN TKKKKLIKCI KNHENDFNKI |
| 121 | CMDMKNYGTN LFEQLSCYNN NFCNTNGIRY HYDEYIHKLI LSVKSKNLNK DLSDMTNILQ |
| 181 | QSELLLTNLN KKMGSYIYID TIKFIHKEMK HIFNRIEYHT KIINDKTKII QDKIKLNIWR |
| 241 | TFQKDELLKR ILDMSNEYSL FITSDHLRQM LYNTFYSKEK HLNNIFHHLI YVLQMKFNDV |
| 301 | PIKMEYFQTY KKNKPLTQ |

PfRH5 amino acid sequence (7G8) excluding signal sequence, flexible N-terminal (amino acids 1 to 159) and flexible loop (amino acids 248 to 296) regions: SEQ ID NO: 10

| | |
|---|---|
| 1 | SIDILQEKEG HLDFVIIPHY TFLDYYKHLS YNSIYHKSST YGKYIAVDAF IKKINEAYDK |
| 61 | VKSKCNDIKN DLIATIKKLE HPYDINNKNR AFKKMMDEYN TKKKKLIKCI KNHENDFNKI |
| 121 | CMDMKNYGTN LFEQLSCYNN NFCNTNGIRY HYDEYIHKLI LSVKSKNLNK DLSDMTNILQ |
| 181 | QSELLLNLN KKMGSYIYID TIKFIHKEMK HIFNRIEYHT KIINDKTKII QDKIKLNIWR |
| 241 | TFQKDELLKR ILDMSNEYSL FITSDHLRQM LYNTFYSKEK HLNNIFHHLI YVLQMKFNDV |
| 301 | PIKMEYFQTY KKNKPLTQ |

Sv2 vaccine sequence based on 3D7 sequence lacking flexible N-terminal region (amino acids 1 to 139) and comprising a Hexa-histidine C-terminal tag (-underlined) and Bip leader sequence (dash-underlined): SEQ ID NO: 11

| | |
|---|---|
| 1 | 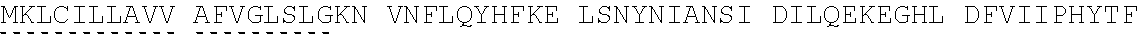 |
| 61 | LDYYKHLSYN SIYHKSSTYG KCIAVDAFIK KINEAYDKVK SKCNDIKNDL IATIKKLEHP |
| 121 | YDINNKNDDS YRYDISEEID DKSEETDDET EEVEDSIQDT DSNHAPSNKK KNDLMNRAFK |
| 181 | KMMDEYNTKK KKLIKCIKNH ENDFNKICMD MKNYGTNLFE QLSCYNNNFC NTNGIRYHYD |
| 241 | EYIHKLILSV KSKNLNKDLS DMTNILQQSE LLLTNLNKKM GSYIYIDTIK FIHKEMKHIF |
| 301 | NRIEYHTKII NDKTKIIQDK IKLNIWRTFQ KDELLKRILD MSNEYSLFIT SDHLRQMLYN |
| 361 | TFYSKEKHLN NIFHHLIYVL QMKFNDVPIK MEYFQTYKKN KPLTQHHHHH H |

Sv2 vaccine sequence based on 7G8 sequence lacking flexible N-terminal region (amino acids 1 to 139) and comprising a Hexa-histidine C-terminal tag (underlined) and Bip leader sequence (dash-underlined): SEQ ID NO: 12

| | |
|---|---|
| 1 | 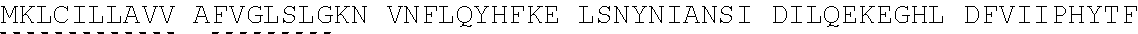 |
| 61 | LDYYKHLSYN SIYHKSSTYG KYIAVDAFIK KINEAYDKVK SKCNDIKNDL IATIKKLEHP |
| 121 | YDINNKNDDS YRYDISEEID DKSEETDDET EEVEDSIQDT DSNHAPSNKK KNDLMNRAFK |
| 181 | KMMDEYNTKK KKLIKCIKNH ENDFNKICMD MKNYGTNLFE QLSCYNNNFC NTNGIRYHYD |
| 241 | EYIHKLILSV KSKNLNKDLS DMTNILQQSE LLLTNLNKKM GSYIYIDTIK FIHKEMKHIF |
| 301 | NRIEYHTKII NDKTKIIQDK IKLNIWRTFQ KDELLKRILD MSNEYSLFIT SDHLRQMLYN |
| 361 | TFYSKEKHLN NIFHHLIYVL QMKFNDVPIK MEYFQTYKKN KPLTQHHHHH H |

Sv3 vaccine sequence based on 3D7 sequence lacking flexible N-terminal (amino acids 1 to 139) and flexible loop (amino acids 248 to 296) regions and comprising a Hexa-histidine C-terminal tag (underlined) and Bip leader sequence (dash-underlined): SEQ ID NO: 13

| | |
|---|---|
| 1 | 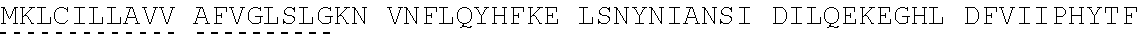 |
| 61 | LDYYKHLSYN SIYHKSSTYG KCIAVDAFIK KINEAYDKVK SKCNDIKNDL IATIKKLEHP |
| 121 | YDINNKNRAF KKMMDEYNTK KKKLIKCIKN HENDFNKICM DMKNYGTNLF EQLSCYNNNF |
| 181 | CNTNGIRYHY DEYIHKLILS VKSKNLNKDL SDMTNILQQS ELLLTNLNKK MGSYIYIDTI |
| 241 | KFIHKEMKHI FNRIEYHTKI INDKTKIIQD KIKLNIWRTF QKDELLKRIL DMSNEYSLFI |
| 301 | TSDHLRQMLY NTFYSKEKHL NNIFHHLIYV LQMKFNDVPI KMEYFQTYKK NKPLTQHHHH |
| 361 | HH |

Sv3 vaccine sequence based on 7G8 sequence lacking flexible N-terminal (amino acids 1 to 139) and flexible loop (amino acids 248 to 296) regions and comprising a Hexa-histidine C-terminal tag (underlined) and Bip leader sequence (dash-underlined): SEQ ID NO: 14

| | |
|---|---|
| 1 | 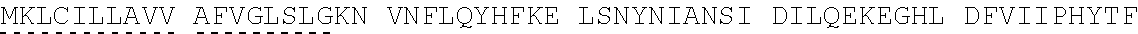 |
| 61 | LDYYKHLSYN SIYHKSSTYG KYIAVDAFIK KINEAYDKVK SKCNDIKNDL IATIKKLEHP |
| 121 | YDINNKNRAF KKMMDEYNTK KKKLIKCIKN HENDFNKICM DMKNYGTNLF EQLSCYNNNF |
| 181 | CNTNGIRYHY DEYIHKLILS VKSKNLNKDL SDMTNILQQS ELLLTNLNKK MGSYIYIDTI |
| 241 | KFIHKEMKHI FNRIEYHTKI INDKTKIIQD KIKLNIWRTF QKDELLKRIL DMSNEYSLFI |
| 301 | TSDHLRQMLY NTFYSKEKHL NNIFHHLIYV LQMKFNDVPI KMEYFQTYKK NKPLTQHHHH |
| 361 | HH |

### Exemplary Modified PfRH5 Antigens

Modified Full Length PfRH5 Amino Acid Sequence (3D7) including Signal Sequence: SEQ ID NO: 15

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNLANS IDILQEKEGH LDFVIIPHYT |
| 181 | FL [symbol] YYKHLSY NSIYHKSSTY GKCIAVDAFI KKINETYDKV KSKCNDIKND LI [symbol] TIKKLEH |
| 241 | PYDINNKNDD SYRYDISEEI DDKSEETDDE TEEVEDSIQD TDSNHTPSNK KENDLMNRTF |
| 301 | KKMFDEYNTK K [symbol] K [symbol] INCIKN HENDFNKIC [symbol] DMKNYGTNLF EQLSCYNNNF CNTNGIRYHY |
| 361 | DEYIHKLIL [symbol] VKSKNLNKDL [symbol] DM [symbol] NILQQS E [symbol] LL [symbol] NL [symbol] KK MGSYIYIDTI KFIHKEMKHI |
| 421 | FNRIEYHTKI INDKTKIIQD KIKLNIWRTF QKDELLK [symbol] IL DMSKEY [symbol] LFI TSDHLRQMLY |
| 481 | NTFYSKEKHL NNIFHHLIYV LQMK [symbol] NDVPI KMEYFQTYKK NKPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS1}) compared with corresponding unmodified PfRH5 antigen are shown in bold.

Modified Full Length PfRH5 Amino Acid Sequence (7C8) including Signal Sequence: SEQ ID NO: 16

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNLANS IDILQEKEGH LDFVIIPHYT |
| 181 | FL [symbol] YYKHLSY NSIYHKSSTY GKCIAVDAFI KKINETYDKV KSKCNDIKND LI [symbol] TIKKLEH |
| 241 | PYDINNKNDD SYRYDISEEI DDKSEETDDE TEEVEDSIQD TDSNHTPSNK KENDLMNRTF |
| 301 | KKM [symbol] DEYNTK K [symbol] K [symbol] I [symbol] CIKN HENDFNKIC [symbol] DMKNYGTNLF EQLSCYNNNF CNTNGIRYHY |
| 361 | DEYIHKLIL [symbol] VKSKNLNKDL [symbol] DM [symbol] NILQQS E [symbol] LL [symbol] NL [symbol] KK MGSYIYIDTI KFIHKEMKHI |
| 421 | FNRIEYHTKI INDKTKIIQD KIKLNIWRTF QKDELLK [symbol] IL DMS [symbol] EY [symbol] LFI TSDHLRQMLY |
| 481 | NTFYSKEKHL NNIFHHLIYV LQMK [symbol] NDVPI KMEYFQTYKK NKPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS1}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 139): SEQ ID NO: 17

| | |
|---|---|
| 1 | KNVNFLQYHF KELSNYN [symbol] AN SIDILQEKEG HLDFVIIPHY TFL [symbol] YYKHLS YNSIYEKSST |
| 61 | YGKCIAVDAF IKKINETYDK VKSKCNDIKN DLI [symbol] TIKKLE HPYDINNKND DSYRYDISEE |
| 121 | IDDKSEETDD ETEEVEDSIQ DTDSNHTPSN KKKNDLMNRT FKKMFDEYNT KK [symbol] K [symbol] I [symbol] CIK |
| 181 | NHENDFNKIC [symbol] DMKNYGTNL FEQLSCYNNN FCNTNGIRYH YDEYIHKLIL [symbol] VKSKNLNKD |
| 241 | L [symbol] DM [symbol] NILQQ SE [symbol] LL [symbol] NL [symbol] K KMGSYIYIDT IKFIHKEMEH IFNRIEYHTK IINDKTKIIQ |
| 301 | DKIKLNIWRT FQKDELLK [symbol] I LDMS [symbol] EY [symbol] LF ITSDHLRQML YNTFYSKEKH LNNIFHHLIY |
| 361 | VLQMK [symbol] NDVP IKMEYFQTYK KNKPLTQ |

Flexible loop (corresponding to amino acids 248 to 296 of full length PfRH5 of SEQ ID NO: 1) region is underlined.

Modified residues (as for PfRH5ΔNL_{HS1}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 139): SEQ ID NO: 18

| | |
|---|---|
| 1 | KNVNFLQYHF KELSNYN [symbol] AN SIDILQEKEG HLDFVIIPHY TFL [symbol] YYKHLS YNSIYEKSST |
| 61 | YGKCIAVDAF IKKINETYDK VKSKCNDIKN DLI [symbol] TIKKLE HPYDINNKND DSYRYDISEE |
| 121 | IDDKSEETDD ETEEVEDSIQ DTDSNHTPSN KKKNDLMNRT FKKM [symbol] DEYNT KK [symbol] K [symbol] INCIK |
| 181 | NHENDFNKIC [symbol] DMKNYGTNL FEQLSCYNNN FCNTNGIRYH YDEYIHKLIL [symbol] VKSKNLNKD |
| 241 | LNDM [symbol] NILQQ SEKLL [symbol] NL [symbol] K KMGSYIYIDT IKFIHKEMEH IFNRIEYHTK IINDKTKIIQ |
| 301 | DKIKLNIWRT FQKDELLK [symbol] I LDMS [symbol] EY [symbol] LF ITSDHLRQML YNTFYSKEKH LNNIFHHLIY |
| 361 | VLQMK [symbol] NDVP IKMEYFQTYK KNKPLTQ |

Flexible loop (corresponding to amino acids 248 to 296 of full length PfRH5 of SEQ ID NO: 2) region is underlined.

Modified residues (as for PfRH5ΔNL_{HS1}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 159): SEQ ID NO: 19

| | |
|---|---|
| 1 | SIDILQEKEG HLDFVIIPHY TFL [symbol] YYKHLS YNSIYHKSST YGKCIAVDAF IKKINETYDK |
| 61 | VKSKCNDIKN DLI [symbol] TIKKLE HPYDINNKND DSYRYDISEE IDDKSEETDD ETEEVEDSIQ |
| 121 | DTDSNHTPSN KKKNDLMNRT FKKMFDEYNT KKNK [symbol] INCIK NHENDFNKIC [symbol] DMKNYGTNL |
| 181 | FEQLSCYNNN FCNTNGIRYH YDEYIHKLIL [symbol] VKSKNLNKD L [symbol] DM [symbol] NILQQ SE [symbol] LII [symbol] NLEK |
| 241 | KMGSYIYIDT IKFIHKEMKH IFNRIEYHTK IINDKTKIIQ DKIKLNIWRT FQKDELLKKI |
| 301 | IDMS [symbol] EY [symbol] LF ITSDHLRQML YNTFYSKEKH LNNIFHHLIY VLQMK [symbol] NDVP IKMEYFQTYK |
| 361 | KNKPLTQ |

Flexible loop (corresponding to amino acids 248 to 296 of full length PfRH5 of SEQ ID NO: 1) region is underlined.

Modified residues (as for PfRH5ΔNL_{HS1}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 159): SEQ ID NO: 20

| | |
|---|---|
| 1 | SIDILQEKEG HLDFVIIPHY TFL [symbol] YYKHLS YNSIYHKSST YGKYIAVDAF IKKINETYDK |
| 61 | VKSKCNDIKN DLI [symbol] TIKKLE HPYDINNKND DSYRYDISEE IDDKSEETDD ETEEVEDSIQ |
| 121 | DTDSNHTPSN KKKNDLMNRT FKKM [symbol] DEYNT KK [symbol] K [symbol] I [symbol] CIK NHENDFNKIC [symbol] DMKNYGTNL |
| 181 | FEQLSCYNNN FCNTNGIRYH YDEYIHKLIL [symbol] VKSKNLNKD L [symbol] DM [symbol] NILQQ SE [symbol] LII [symbol] NL [symbol] K |
| 241 | KMGSYIYIDT IKFIHKEMKH IFNRIEYHTK IINDKTKIIQ DKIKLNIWRT FQKDELLK [symbol] I |
| 301 | LDMS [symbol] EY [symbol] LF ITSDHLRQML YNTFYSKEKH LNNIFHHLIY VLQMK [symbol] NDVP IKMEYFQTYK |
| 361 | KNKPLTQ |

Flexible loop (corresponding to amino acids 248 to 296 of full length PfRH5 of SEQ ID NO: 2) region is underlined.

Modified residues (as for PfRH5ΔNL_{HS1}) compared with corresponding unmodified PfRH5 antigen are shown in bold.

### PfRH5ΔNL_{HS1} Excluding Signal Sequence, Flexible N-Terminal (Amino Acids 1 to 139) and Flexible Loop (Amino Acids 248 to 296): SEQ ID NO: 21

KNVNFLQYHFKELSNYN [symbol] ANSIDILQEKEGHLDFVIIPHYTFL [symbol] YYKHLS
YNSIYHKSSTYGKYIAVDAFIKKINEAYDKVKSKCNDIKNDLI [symbol] TIKKLE
HPYDINNKNRAFKKM [symbol] DEYNTKK [symbol] K [symbol] I [symbol] CIKNHENDFNKIC [symbol] DMKNYGTN
LFEQLSCYNNNFCNTNGIRYHYDEYIHKLIL [symbol] VKSKNLNKDL [symbol] DM [symbol] NILQ
QSE [symbol] LL [symbol] NL [symbol] KKMGSYIYIDTIKFIHKEMKHIFNRIEYHTKIINDKTKII
QDKIKLDIWRTFQKDELLK [symbol] ILDMS [symbol] EY [symbol] LFITSDDLRQMLYNTFYSKEK
HLNNIFHHLIYVLQMK [symbol] NDVPIKMEYFQTYKKNKPLTQ

Modified residues compared with corresponding unmodified PfRH5 antigen are shown in bold and italicised.

PfRH5ΔNL_{HS1} Excluding Signal Sequence, Flexible N-Terminal (Amino Acids 1 to 159) and Flexible Loop (Amino Acids 248 to 296): SEQ ID NO: 22

SIDILQEKEGHLDFVIIPHYTFL [symbol] YYKHLSYNSIYHKSSTYGKYIAVDAF
IKKINETYDKVKSKCNDIKNDLI [symbol] TIKKLEHPYDINNKNRAFKKMMDEYN
TKKNK [symbol] I [symbol] CIKNHENDFNKIC [symbol] DMKNYGTNLFEQLSCYNNNFCNTNGIRY
HYDEYIHKLIL [symbol] VKSKNLNKDL [symbol] DM [symbol] NILQQSE [symbol] LL [symbol] NL [symbol] KKMGSYIYID
TIKFIHKEMKHIFNRIEYHTKIINDKTKIIQDKIKLNIWRTFQKDELLKK
ILDMS [symbol] EY [symbol] LFITSDDLRQMLYNTFYSKEKHLNNIFHHLIYVLQMK [symbol] NDV
PIKMEYFQTYKKNKPLTQ

Modified residues compared with corresponding unmodified PfRH5 antigen are shown in bold and italicised.

PfRH5ΔNL_{HS1} Excluding Signal Sequence, Flexible N-Terminal (Amino Acids 1 to 139) and Flexible Loop (Amino Acids 248 to 296) and comprising a Hexa-Histidine C-Terminal Tag (Underlined): SEQ ID NO: 23

KNVNFLQYHFKELSNYN [symbol] ANSIDILQEKEGHLDFVIIPHYTFL [symbol] YYKHLS
YNSIYHKSSTYGKYIAVDAFIKKINEAYDKVKSKCNDIKNDLI [symbol] TIKKLE
HPYDINNKNRAFKKM [symbol] DEYNTKK [symbol] K [symbol] I [symbol] CIKNHENDFNKIC [symbol] DMKNYGTN
LFEQLSCYNNNFCNTNGIRYHYDEYIHKLIL [symbol] VKSKNLNKDL [symbol] DM [symbol] NILQ
QSE [symbol] LL [symbol] NL [symbol] KKMGSYIYIDTIKFIHKEMKHIFNRIEYHTKIINDKTKII
QDKIKLNIWRTFQKDELLKKILDMS [symbol] EY [symbol] LFITSDDLRQMLYNTFYSKEK
HLNNIFHHLIYVLQMK [symbol] NDVPIKMEYFQTYKKNKPLTQHHHHHH

Modified residues compared with corresponding unmodified PfRH5 antigen are shown in bold and italicised.

PfRH5ΔNL_{HS1} Excluding Signal Sequence, Flexible N-Terminal (Amino Acids 1 to 159) and Flexible Loop (Amino Acids 248 to 296) and comprising a Hexa-Histidine C-Terminal Tag (Underlined): SEQ ID NO: 24

SIDILQEKEGHLDFVIIPHYTFL [symbol] YYKHLSYNSIYHKSSTYGKYIAVDAF
IKKINETYDKVKSKCNDIKNDLI [symbol] TIKKLEHPYDINNKNRAFKKM [symbol] DEYN
TKK [symbol] K [symbol] I [symbol] CIKNHENDFNKIC [symbol] DMKNYGTNLFEQLSCYNNNFCNTNGIRY
HYDEYIHKLIL [symbol] VKSKNLNKDL [symbol] DM [symbol] NILQQSE [symbol] LL [symbol] NL [symbol] KKMGSYIYID
TIKFIHKEMKHIFNRIEYHTKIINDKTKIIQDKIKLNIWRTFQKDELLK [symbol]
ILDMS [symbol] EY [symbol] LFITSDHLRQMLYNTFYSKEKHLNNIFHHLIYVLQMK [symbol] NDV
PIKMEYFQTYKKNKPLTQHHHHHH

Modified residues compared with corresponding unmodified PfRH5 antigen are shown in bold and italicised.

PfRH5ΔNL_{HS1} Excluding Signal Sequence, Flexible N-Terminal (Amino Acids 1 to 139) and Flexible Loop (Amino Acids 248 to 296) and comprising a Bip Leader Sequence (Dotted Underlined): SEQ ID NO: 25

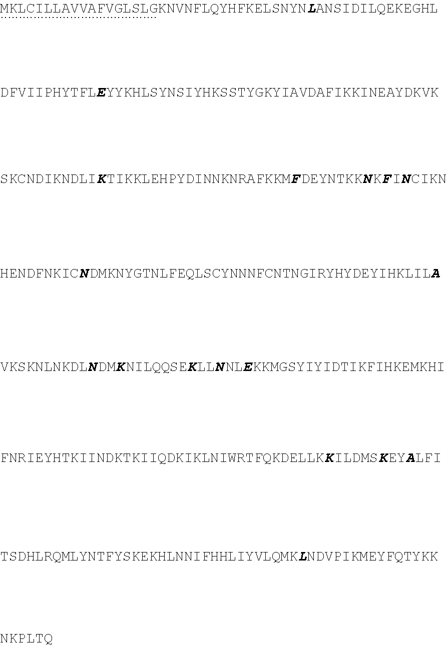

Modified residues compared with corresponding unmodified PfRH5 antigen are shown in bold and italicised.

PfRH5ΔNL_{HS1} Excluding Signal Sequence, Flexible N-Terminal (Amino Acids 1 to 159) and Flexible Loop (Amino Acids 248 to 296) and comprising a Bip Leader Sequence (Dotted Underlined): SEQ ID NO: 26

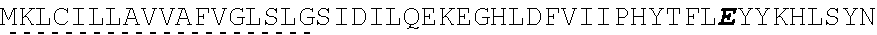

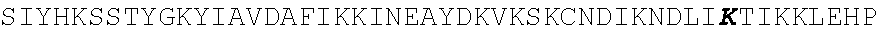

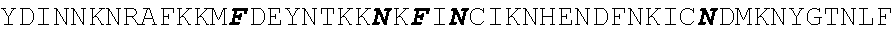

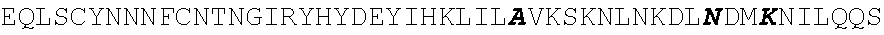

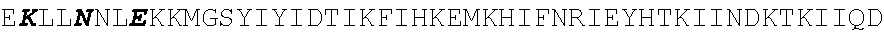

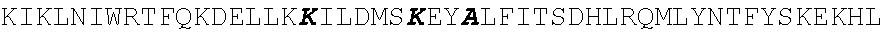

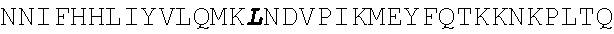

Modified residues compared with corresponding unmodified PfRH5 antigen are shown in bold and italicised.

PfRH5ΔNL_{HS1} Excluding Signal Sequence, Flexible N-Terminal (Amino Acids 1 to 139) and Flexible Loop (Amino Acids 248 to 296) and comprising a Bip Leader Sequence (Dotted Underlined) and a Hexa-Histidine C-Terminal Tag (Underlined): SEQ ID NO: 27

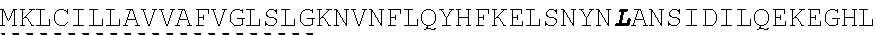

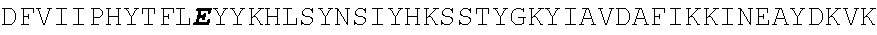

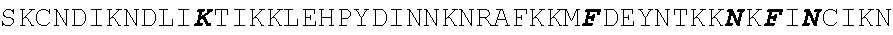

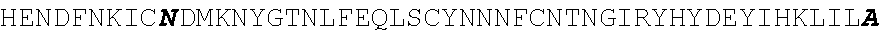

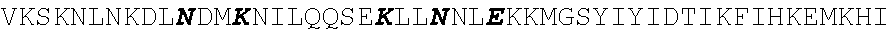

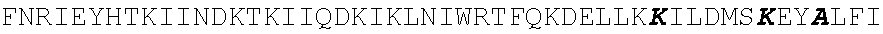

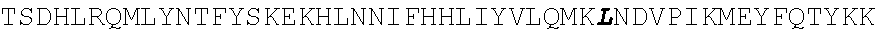

NKPLTQHHHHHH

PfRH5ΔNL_{HS1} Excluding Signal Sequence, Flexible N-Terminal (Amino Acids 1 to 159) and Flexible Loop (Amino Acids 248 to 296) and comprising a Bip Leader Sequence (Dotted Underlined) and a Hexa-Histidine C-Terminal Tag (Underlined): SEQ ID NO: 28

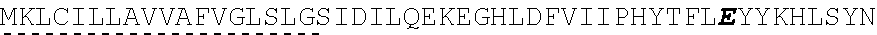

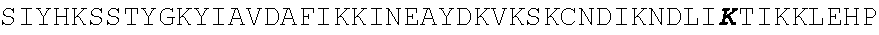

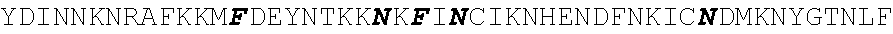

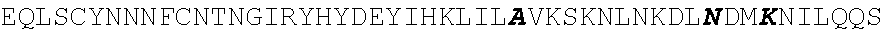

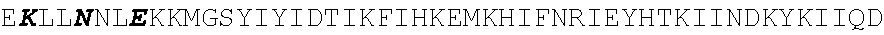

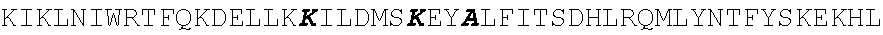

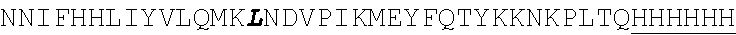

Modified residues compared with corresponding unmodified PfRH5 antigen are shown in bold and italicised.

Modified Full Length PfRH5 Amino Acid sequence (3D7) including Signal Sequence: SEQ ID NO: 29

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FL [symbol] YYKHLSY I [symbol] IYHKSSTY GKCIAVDAFI KKINETYDKV KSKCNDIKND LI [symbol] TI [symbol] KLEH |
| 241 | PYDINNKNDD SYRYDISEEI DDKSEETDDE TEEVEDSIQD TDSNHTPSNK KKNDLMNRTF |
| 301 | KKMMDEY [symbol] TK KKK [symbol] IKCIKN HENDFNKICM DMKNYGTNLF EQLSCYNNNF CNTNGIRYHY |
| 361 | DEYIHKLI [symbol] [symbol] VKSKNLNKDL [symbol] DM [symbol] NILQQS E [symbol] LL [symbol] NL [symbol] KK MGSYIYIDTI KFI [symbol] KEMKHI |
| 421 | FNRIEYHTKI INDKTKIIQD KIK [symbol] [symbol] IWRTF QKDELLKRIL DM [symbol] [symbol] EY [symbol] LF [symbol] TSD [symbol] LRQMLY |
| 481 | NTFYSKEKHL NNIF [symbol] HLIYV LQMK [symbol] NDVPI [symbol] MEYFQTYKK NKPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS2}) compared with corresponding unmodified PfRH5 antigen are shown in bold.

Modified Full Length PfRH5 Amino Acid Sequence (7G8) including Signal Sequence: SEQ ID NO: 30

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FL [symbol] YYKHLSY [symbol] [symbol] IYHKSSTY GKYIAVDAFI KKINETYDKV KSKCNDIKND LI [symbol] TI [symbol] KLEH |
| 241 | PYDINNKNDD SYRYDISEEI DDKSEETDDE TEEVEDSIQD TDSNHTPSNK KKNDLMNRTF |
| 301 | KKMMDEY [symbol] KTK KKK [symbol] IKCIKN HENDFNKICM DMKNYGTNLF EQLSCYNNNF CNTNGIRYHY |
| 361 | DEYIHKLI [symbol] [symbol] VKSKNLNKDL [symbol] DM [symbol] NILOQS E [symbol] LL [symbol] NL [symbol] KK MGSYIYIDTI KFI [symbol] KEMKHI |
| 421 | FNRIEYHTKI INDKTKIIQD KIK [symbol] [symbol] IWRTF QKDELLKRIL DM [symbol] [symbol] EY [symbol] LF [symbol] TSD [symbol] LRQMLY |
| 481 | NTFYSKEKHL NNIF [symbol] HLIYV LQMK [symbol] NDVPI [symbol] MEYFQTYKK NKPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS2}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 139): SEQ ID NO: 31

| | |
|---|---|
| 1 | KNVNFLQYHF KELSNYNIAN SIDILQEKEG HLDFVIIPHY TFL [symbol] YYKHLS Y [symbol] [symbol] IYHKSST |
| 61 | YGKCIAVDAF IKKINETYDK VKSKCNDIKN DLI [symbol] TI [symbol] KLE HPYDINNKND DSYRYDISEE |
| 121 | IDDKSEETDD ETEEVEDSIQ DTDSNHTPSN KKKNDLMNRT FKKMMDEY [symbol] T KKKK [symbol] IKCIK |
| 181 | NHENDFNKIC MDMKNYGTNL FEQLSCYNNN FCNTNGIRYH YDEYIHKLI [symbol] [symbol] VKSKNLNKD |
| 241 | L [symbol] DM [symbol] NILQQ SE [symbol] LL [symbol] NL [symbol] K KMGSYIYIDT IKFI [symbol] KEMKH IFNRIEYHTK IINDKTKIIQ |
| 301 | DKIK [symbol] [symbol] IWRT FQKDELLKRI LDM [symbol] [symbol] EY [symbol] LF [symbol] TSD [symbol] LRQML YNTFYSKEKH LNNIF [symbol] HLIY |
| 361 | VLQMK [symbol] NDVP I [symbol] MEYFQTYK KNKPLTQ |

Flexible loop (corresponding to amino acids 248 to 296 of full length PfRH5 of SEQ ID NO: 1) region is underlined.

Modified residues (as for PfRH5ΔNL_{HS2}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 139): SEQ ID NO: 32

| | |
|---|---|
| 1 | KNVNFLQYHF KELSNYNIAN SIDILQEKEG HLDFVIIPHY TFL [symbol] YYKHLS Y [symbol] [symbol] IYHKSST |
| 61 | YGKYIAVDAF IKKINETYDK VKSKCNDIKN DLI [symbol] TI [symbol] KLE HPYDINNKND DSYRYDISEE |
| 121 | IDDKSEETDD ETEEVEDSIQ DTDSNHTPSN KKKNDLMNRT FKKMMDEY [symbol] T KKKK [symbol] IKCIK |
| 181 | NHENDFNKIC MDMKNYGTNL FEQLSCYNNN FCNTNGIRYH YDEYIHKLI [symbol] [symbol] VKSKNLNKD |
| 241 | L [symbol] DM [symbol] NILQQ SE [symbol] LL [symbol] NL [symbol] K KMGSYIYIDT IKFI [symbol] KEMKH IFNRIEYHTK IINDKTKIIQ |
| 301 | DKIK [symbol] [symbol] IWRT FQKDELLKRI LDM [symbol] [symbol] EY [symbol] LF [symbol] TSD [symbol] LRQML YNTFYSKEKH LNNIF [symbol] HLIY |
| 361 | VLQMK [symbol] NDVP I [symbol] MEYFQTYK KNKPLTQ |

Flexible loop (corresponding to amino acids 248 to 296 of full length PfRH5 of SEQ ID NO: 2) region is underlined.

Modified residues (as for PfRH5ΔNL_{HS2}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 159): SEQ ID NO: 33

| | |
|---|---|
| 1 | SIDILQEKEG HLDFVIIPHY TFL [symbol] YYKHLS Y [symbol] [symbol] IYHKSST YGKCIAVDAF IKKINETYDK |
| 61 | VKSKCNDIKN DLI [symbol] TI [symbol] KLE HPYDINNKND DSYRYDISEE IDDKSEETDD ETEEVEDSIQ |
| 121 | DTDSNHTPSN KKKNDLMNRT FKKMMDEY [symbol] T KKKK [symbol] IKCIK NHENDFNKIC MDMKNYGTNL |
| 181 | FEQLSCYNNN FCNTNGIRYH YDEYIHKLI [symbol] [symbol] VKSKNLNED L [symbol] DM [symbol] NILQQ SE [symbol] LL [symbol] NLKK |
| 241 | KMGSYIYIDT IKFI [symbol] KEMKH IFNRIEYHTK IINDKTKIIQ DKIK [symbol] [symbol] IWRT FQKDELLKRI |
| 301 | LDM [symbol] [symbol] EY [symbol] LF [symbol] TSD [symbol] LRQML YNTFYSKEKH LNNIF [symbol] HLIY VLQMK [symbol] NDVP I [symbol] MEYFQTYK |
| 361 | KNKPLTQ |

Flexible loop (corresponding to amino acids 248 to 296 of full length PfRH5 of SEQ ID NO: 1) region is underlined.

Modified residues (as for PfRH5ΔNL_{HS2}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 159): SEQ ID NO: 34

| | |
|---|---|
| 1 | SIDILQEKEG HLDFVIIPHY TFL [symbol] YYKHLS Y [symbol] [symbol] IYHKSST YGKYIAVDAF IKKINETYDK |
| 61 | VYSKCNDIKN DLI [symbol] TI [symbol] KLE HPYDINNKND DSYRYDISEE IDDKSEETDD ETEEVEDSIQ |
| 121 | DTDSNHTPSN KKKNDLMNRT FKKMMDEY [symbol] T KKKK [symbol] IKCIK NHENDFNKIC MDMKNYGTNL |
| 181 | FEQLSCYNNN FCNTNGIRYH YDEYIHKLI [symbol] [symbol] VKSKNLNKD L [symbol] DM [symbol] NILQQ SE [symbol] LL [symbol] NL [symbol] K |
| 241 | KMGSYIYIDT IKFI [symbol] KEMKH IFNRIEYHTK IINDKTKIIQ DKIK [symbol] [symbol] IWRT FQKDELLKRI |
| 301 | LDM [symbol] [symbol] EY [symbol] LF [symbol] TSD [symbol] LRQML YNTFYSKEKH LNNIF [symbol] HLIY VLQMK [symbol] NDVP I [symbol] MEYFQTYK |
| 361 | KNKPLTQ |

Flexible loop (corresponding to amino acids 248 to 296 of full length PfRH5 of SEQ ID NO: 2) region is underlined.

Modified residues (as for PfRH5ΔNL_{HS2}) compared with corresponding unmodified PfRH5 antigen are shown in bold.

### PfRH5ΔNL_{HS2} Excluding Signal Sequence, Flexible N-Terminal (Amino Acids 1 to 139) and Flexible Loop (Amino Acids 248 to 296): SEQ ID NO: 35

KNVNFLQYHEKELSNYNIANSIDILQEKEGHLDFVIIPHYTFL [symbol] YYKHLS
Y [symbol] [symbol] IYHKSSTYGKYIAVDAFIKKINEAYDKVKSKCNDIKNDLI [symbol] TI [symbol] KLE
HPYDINNKNRAFKKMMDEY [symbol] TKKKK [symbol] IKCIKNHENDFNKICMDMKNYGTN
LFEQLSCYNNNFCNTNGIRYHYDEYIHKLI [symbol] [symbol] VKSKNLNKDL [symbol] DM [symbol] NILQ
QSE [symbol] LL [symbol] NL [symbol] KKMGSYIYIDTIKFI [symbol] KEMKHIFNRIEYHTKIINDKTKII
QDKIK [symbol] [symbol] IWRTFQKDELLKRILDM [symbol] [symbol] EY [symbol] LF [symbol] TSD [symbol] LRQMLYNTFYSKEK
HLNNIF [symbol] HLIYVLQMK [symbol] NDVPI [symbol] MEYFQTYKKNKPLTQ

Modified residues compared with corresponding unmodified PfRH5 antigen are shown in bold and italicised.

### PfRH5ΔNL_{HS1} Excluding Signal Sequence, Flexible N-Terminal (Amino Acids 1 to 159) and Flexible Loop (Amino Acids 248 to 296): SEQ ID NO: 36

SIDILQEKEGHLDFVIIPHYTFL [symbol] YYKHLSY [symbol] [symbol] IYHKSSTYGKYIAVDAF
IKKINEAYDKVKSKCNDIKNDLI [symbol] TI [symbol] KLEHPYDINNKNRAFKKMMDEY [symbol]
TKKKK [symbol] IKCIKNHENDFNKICMDMKNYGTNLFEQLSCYNNNFCNTNGIRY
HYDEYIHKLI [symbol] [symbol] VKSKNLNKDL [symbol] DM [symbol] NILQQSE [symbol] LL [symbol] NL [symbol] KMGSYIYIDT
IKFI [symbol] KEMKHIFNRIEYHTKIINDKTKIIQDKIK [symbol] [symbol] IWRTFQKDELLKRI
LDM [symbol] [symbol] EY [symbol] LF [symbol] TSD [symbol] LRQMLYNTFYSKEKHLNNIF [symbol] HLIYV [symbol] QMKLNDVP
I [symbol] MEYFQTYKKNKPLTQ

Modified residues compared with corresponding unmodified PfRH5 antigen are shown in bold and italicised.

PfRH5ΔNL_{HS2} Excluding Signal Sequence, Flexible N-Terminal (Amino Acids 1 to 139) and Flexible Loop (Amino Acids 248 to 296) and comprising a Hexa-Histidine C-Terminal Tag (Underlined): SEQ ID NO: 37

KNVNFLQYHEKELSNYNIANSIDILQEKEGHLDFVIIPHYTFL [symbol] YYKHLS
Y [symbol] [symbol] IYHKSSTYGKYIAVDAFIKKINEAYDKVKSKCNDIKNDLI [symbol] TI [symbol] KLE
HPYDINNKNRAFKKMMDEY [symbol] TKKKK [symbol] IKCIKNHENDFNKICMDMKNYGTN
LFEQLSCYNNNFCNTNGIRYHYDEYIHKLI [symbol] [symbol] VKSKNLNKDL [symbol] DM [symbol] NILQ
QSE [symbol] LL [symbol] NL [symbol] KKMGSYIYIDTIKFI [symbol] KEMKHIFNRIEYHTKIINDKTKII
QDKIK [symbol] [symbol] IWRTFQKDELLKRILDM [symbol] [symbol] EY [symbol] LF [symbol] TSD [symbol] LRQMLYNTFYSKEK
HLNNIF [symbol] HLIYVLQMK [symbol] NDVPI [symbol] MEYFQTYKKNKPLTQHHHHHH

Modified residues compared with corresponding unmodified PfRH5 antigen are shown in bold and italicised.

PfRH5ΔNL_{HS2} Excluding Signal Sequence, Flexible N-Terminal (Amino Acids 1 to 159) and Flexible Loop (Amino Acids 248 to 296) and comprising a Hexa-Histidine C-Terminal Tag (Underlined): SEQ ID NO: 38

SIDILQEKEGHLDFVIIPHYTFL [symbol] YYKHLSY [symbol] [symbol] IYHKSSTYGKIAVDAFI
KKINEAYDKVKSKCNDIKNDLI [symbol] TI [symbol] KLEHPYDINNKNRAFKKMMDEY [symbol] K
KKK [symbol] IKCIKNHENDFNKICMDMKNYGTNLFEQLSCYNNNFCNTNGIRYHY
DEYIHKLI [symbol] [symbol] VKSKNLNKDL [symbol] DM [symbol] NILQQSE [symbol] LL [symbol] NL [symbol] KKMGSYIYIDTI
KFI [symbol] KEMKHIFNRIEYHTKIINDKTKIIQDKIK [symbol] [symbol] IWRTFQKDELLKRIL
DM [symbol] [symbol] EY [symbol] LF [symbol] TSD [symbol] LRQMLYNTFYSKEKHLNNIF [symbol] HLIYVLQMK [symbol] NDVPI [symbol]
MEYFQTYKKNKPLTQHHHHHH

Modified residues compared with corresponding unmodified PfRH5 antigen are shown in bold and italicised.

PfRH5ΔNL_{HS2} Excluding Signal Sequence, Flexible N-Terminal (Amino Acids 1 to 139) and flexible Loop (Amino Acids 248 to 296) and comprising a Bip Leader Sequence (Dotted Underlined): SEQ ID NO: 39

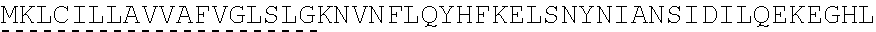

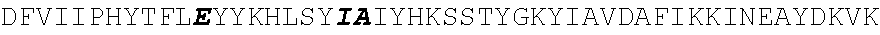

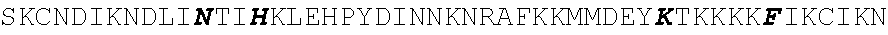

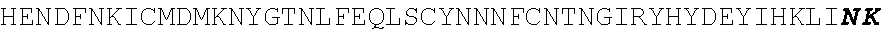

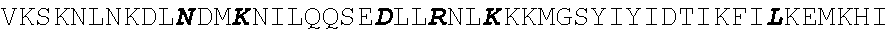

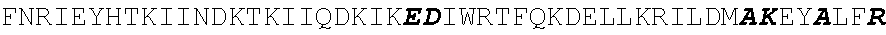

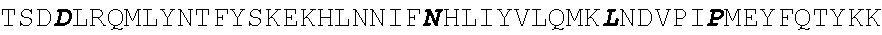

NKPLTQ

Modified residues compared with corresponding unmodified PfRH5 antigen are shown in bold and italicised.

PfRH5ΔNL_{HS2} Excluding Signal Sequence, Flexible N-Terminal (Amino Acids 1 to 159) and Flexible Loop (Amino Acids 248 to 296) and comprising a Bip Leader Sequence (Dotted Underlined): SEQ ID NO: 40

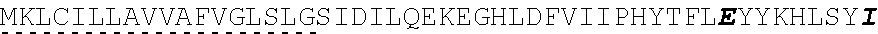

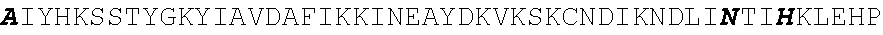

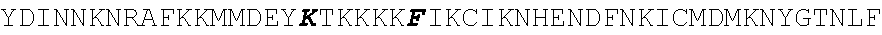

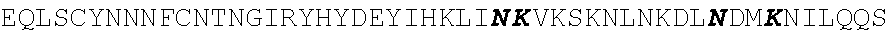

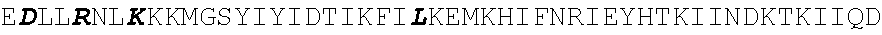

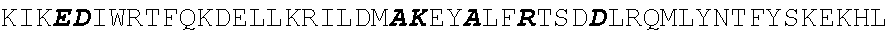

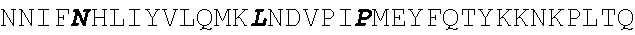

Modified residues compared with corresponding unmodified PfRH5 antigen are shown in bold and italicised.

PfRH5ΔNL_{HS2} Excluding Signal Sequence, Flexible N-Terminal (Amino Acids 1 to 139) and Flexible Loop (Amino Acids 248 to 296) and comprising a Bip Leader Sequence (Dotted Underlined) and a Hexa-Histidine C-Terminal Tag (underlined): SEQ 10 NO: 41

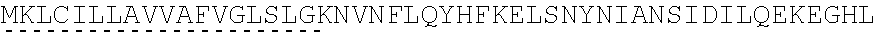

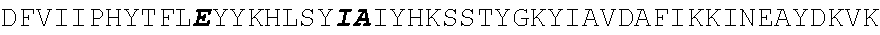

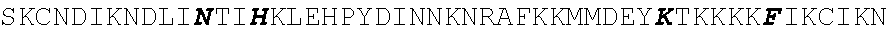

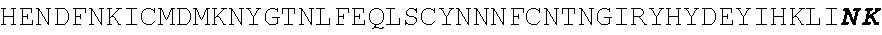

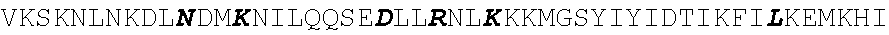

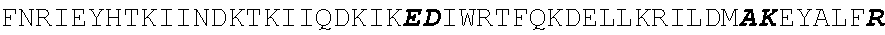

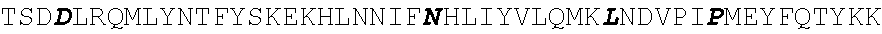

NKPLTQHHHHHH

Modified residues compared with corresponding unmodified PfRH5 antigen are shown in bold and italicised.

PfRH5ΔNL_{HS2} Excluding Signal Sequence, Flexible N-Terminal (Amino Acids 1 to 159) and Flexible Loop (Amino Acids 248 to 296) and comprising a Bip Leader Sequence (Dotted Underlined) and a Hexa-Histidine C-Terminal Tag (Underlined): SEQ ID NO: 42

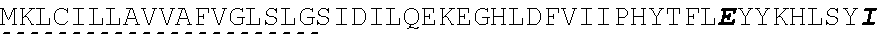

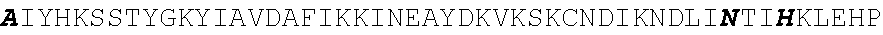

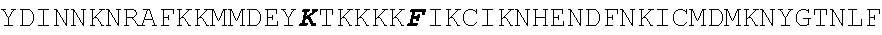

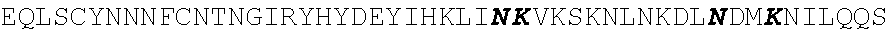

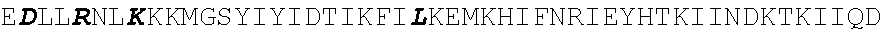

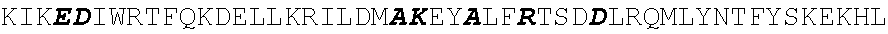

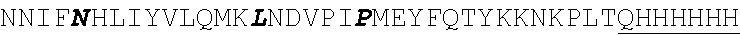

Modified residues compared with corresponding unmodified PfRH5 antigen are shown in bold and italicised.

Modified Full Length PfRH5 Amino Acid Sequence (3D7) including Signal Sequence: SEQ ID NO: 43

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FL [symbol] YYKHLSY [symbol] [symbol] IYHKSSTY GKCIAVDAFI KKINETYDKV KSKCNDIKND LI [symbol] TIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLI [symbol] S VKSKNLNKDL [symbol] DMTNILQQS E [symbol] LLTNL [symbol] KK MGSYIYIDTI KFIHKEMKHI |
| 421 | FNRIEYHTKI INDKTKIIQD KIKL [symbol] IWRTF QKDELLKRIL DM [symbol] [symbol] EY [symbol] LF [symbol] TSD [symbol] LRQMLY |
| 481 | NTFYSKEKHL NNIFHHLIYV LQMKFNDVPI [symbol] MEYFQTYKK NEPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS3}) compared with corresponding unmodified PfRH5 antigen are shown in bold.

Modified Full Length PfRH5 Amino Acid Sequence (7G8) including Signal Sequence: SEQ ID NO: 44

| | |
|---|---|
| 1 | MIRIKKKLIL TIIYIHLFIL NRLSFENAIK KTKNQENNLT LLPIKSTEEE KDDIKNGKDI |
| 61 | KKEIDNDKEN IKTNNAKDHS TYIKSYLNTN VNDGLKYLFI PSHNSFIKKY SVFNQINDGM |
| 121 | LLNEKNDVKN NEDYKNVDYK NVNFLQYHFK ELSNYNIANS IDILQEKEGH LDFVIIPHYT |
| 181 | FL [symbol] YYKHLSY [symbol] [symbol] IYEKSSTY GKYIAVDAFI KKINETYDKV KSKCNDIKND LI [symbol] TIKKLEH |
| 241 | PYDINNKNDD SYRYDISEEI DDKSEETDDE TEEVEDSIQD TDSNHTPSNK KKNDLMNRTF |
| 301 | KKMMDEYNTK KKKLIKCIKN HENDFNKICM DMKNYGTNLF EQLSCYNNNF CNTNGIRYHY |
| 361 | DEYIHKLI [symbol] S VKSKNLNKDL [symbol] DMTNILOQS E [symbol] LLTNL [symbol] KK MGSYIYIDTI KFIHKEMKHI |
| 421 | FNRIEYHTKI INDKTKIIQD KIKL [symbol] IWRTF QKDELLKRIL DM [symbol] [symbol] EY [symbol] LF [symbol] TSD [symbol] LRQMLY |
| 481 | NTFYSKEKHL NNIFHHLIYV LQMKFNDVPI [symbol] MEYFQTYKK NKPLTQ |

Signal sequence (amino acids 1 to 23) is in italics, flexible N-terminal (amino acids 1 to 139) and flexible loop (amino acids 248 to 296) regions are underlined.

Modified residues (as for PfRH5ΔNL_{HS3}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 139): SEQ ID NO: 45

| | |
|---|---|
| 1 | KNVNFLQYHF KELSNYNIAN SIDILQEKEG HLDFVIIPHY TFL [symbol] YYKHLS Y [symbol] [symbol] IYHKSST |
| 61 | YGKCIAVDAF IKKINETYDK VKSKCNDIKN DLI [symbol] TIKKLE HPYDINNKND DSYRYDISEE |
| 121 | IDDKSEETDD ETEEVEDSIQ DTDSNHTPSN KKKNDLMNRT FKKMMDEYNT KKKKLIKCIK |
| 181 | NHENDFNKIC MDMKNYGTNL FEQLSCYNNN FCNTNGIRYH YDEYIHKLI [symbol] SVKSKNLNKD |
| 241 | L [symbol] DMTNILQQ SE [symbol] LLTNL [symbol] K KMGSYIYIDT IKFIHKEMKH IFNRIEYHTK IINDKTKIIQ |
| 301 | DKIKL [symbol] IWRT FQKDELLKRI LDM [symbol] [symbol] EY [symbol] LF [symbol] TSD [symbol] LRQML YNTFYSKEKH LNNIFHHLIY |
| 361 | VLQMKFNDVP I [symbol] MEYFQTYK KNKPLTQ |

Flexible loop (corresponding to amino acids 248 to 296 of full length PfRH5 of SEQ ID NO: 1) region is underlined.

Modified residues (as for PfRH5ΔNL_{HS3}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 139): SEQ ID NO: 46

| | |
|---|---|
| 1 | KNVNFLQYHF KELSNYNIAN SIDILQEKEG HLDFVIIPHY TFL [symbol] YYKHLS Y [symbol] [symbol] IYHKSST |
| 61 | YGKCIAVDAF IKKINETYDK VKSKCNDIKN DLI [symbol] TIKKLE HPYDINNKND DSYRYDISEE |
| 121 | IDDKSEETDD ETEEVEDSIQ DTDSNHTPSN KKKNDLMNRT FKKMMDEYNT KKKKLIKCIK |
| 181 | NHENDFNKIC MDMKNYGTNL FEQLSCYNNN FCNTNGIRYH YDEYIHKLI [symbol] SVKSKNLNKD |
| 241 | L [symbol] DMTNILQQ SE [symbol] LLTNL [symbol] K KMGSYIYIDT IKFIHKEMKH IFNRIEYHTK IINDKTKIIQ |
| 301 | DKIKL [symbol] IWRT FQKDELLKRI LDM [symbol] [symbol] EY [symbol] LF [symbol] TSD [symbol] LRQML YNTFYSKEKH LNNIFHHLIY |
| 361 | VLQMKFNDVP I [symbol] MEYFQTYK KNKPLTQ |

Flexible loop (corresponding to amino acids 248 to 296 of full length PfRH5 of SEQ ID NO: 2) region is underlined.

Modified residues (as for PfRH5ΔNL_{HS3}) compared with corresponding unmodified PfRH5 antigen are shown in bold.

### Modified PfRH5 Amino Acid Sequence (3D7) Excluding Signal Sequence and Flexible N-Terminal Region (Amino Acids 1 to 159): SEQ ID NO: 47

| | |
|---|---|
| 1 | SIDILQEKEG HLDFVIIPHY TFL [symbol] YYKHLS Y [symbol] [symbol] IYHKSST YGKCIAVDAF IKKINETYDK |
| 61 | VKSKCNDIKN DLI [symbol] TIKKLE HPYDINNKND DSYRYDISEE IDDKSEETDD ETEEVEDSIQ |
| 121 | DTDSNHTPSN KKKNDLMNRT FKKMMDEYNT KKKKLIKCIK NHENDFNKIC MDMKNYGTNL |
| 181 | FEQLSCYNNN FCNTNGIRYH YDEYIHKLI [symbol] SVKSKNLNKD L [symbol] DMTNILQQ SE [symbol] LLTNL [symbol] K |
| 241 | KMGSYIYIDT IKFIHKEMKH IFNRIEYHTK IINDKTKIIQ DKIKL [symbol] IWRT FQKDELLKRI |
| 301 | LDMA [symbol] EY [symbol] LF [symbol] TSDDLRQML YNTFYSKEKH LNNIFHHLIY VLQMKFNDVP I [symbol] MEYFQTYK |
| 361 | KNKPLTQ |

Flexible loop (corresponding to amino acids 248 to 296 of full length PfRH5 of SEQ ID NO: 1) region is underlined.

Modified residues (as for PfRH5ΔNL_{HS3}) compared with corresponding unmodified PfRH5 antigen are shown in bold.

### Modified PfRH5 Amino Acid Sequence (7G8) Excluding Signal Sequence and Flexible N-Terminal Region (Amino Acids 1 to 159): SEQ ID NO: 48

| | |
|---|---|
| 1 | SIDILQEKEG HLDFVIIPHY TFL [symbol] YYKHLS Y [symbol] [symbol] IYHKSST YGKYIAVDAF IKKINETYDK |
| 61 | VKSKCNDIKN DLI [symbol] TIKKLE HPYDINNKND DSYRYDISEE IDDKSEETDD ETEEVEDSIQ |
| 121 | DTDSNHTPSN KKKNDLMNRT FKKMMDEYNT KKKKLIKCIK NHENDFNKIC MDMKNYGTNL |
| 181 | FEQLSCYNNN FCNTNGIRYH YDEYIHKLI [symbol] SVKSKNLNKD L [symbol] DMTNILQQ SE [symbol] LLTNL [symbol] K |
| 241 | KMGSYIYIDT IKFIHKEMKH IFNRIEYHTK IINDKTKIIQ DKIKL [symbol] IWRT FQKDELLKRI |
| 301 | LDMA [symbol] EY [symbol] LF [symbol] TSD [symbol] LRQML YNTFYSKEKH LNNIFHHLIY VLQMKFNDVP I [symbol] MEYFQTYK |
| 361 | KNKPLTQ |

Flexible loop (corresponding to amino acids 248 to 296 of full length PfRH5 of SEQ ID NO: 2) region is underlined.

Modified residues (as for PfRH5ΔNL_{HS3}) compared with corresponding unmodified PfRH5 antigen are shown in bold.

### PfRH5ΔNL_{HS3} Excluding Signal Sequence, Flexible N-Terminal (Amino Acids 1 to 139) and Flexible Loop (Amino Acids 248 to 296): SEQ ID NO: 49

KNVNFLQYHFKELSNYNIANSIDILQEKEGHLDFVIIPHYTFL [symbol] YYKHLS
Y [symbol] [symbol] IYHKSSTYGKYIAVDAFIKKINEAYDKVKSKCNDIKNDLI [symbol] TIKKLE
HPYDINNKNRAFKKMMDEYNTKKKKLIKCIKNHENDFNKICMDMKNYGTN
LFEQLSCYNNNFCNTNGIRYHYDEYIHKLI [symbol] SVKSKNLNKDL [symbol] DMTNILQ
QSE [symbol] LLTNL [symbol] KKMGSYIYIDTIKFIHKEMKHIFNRIEYHTKIINDKTKII
QDKIKL [symbol] IWRTFQKDELLKRILDM [symbol] [symbol] EY [symbol] LF [symbol] TSD [symbol] LRQMLYNTFYSKEK
HLNNIFHHLIYVLQMKFNDVPI [symbol] MEYFQTYKKNKPLTQ

Modified residues compared with corresponding unmodified PfRH5 antigen are shown in bold and italicised.

### PfRH5ΔNL_{HS3} Excluding Signal Sequence, Flexible N-Terminal (Amino Acids 1 to 159) and Flexible Loop (Amino Acids 248 to 296): SEQ ID NO: 50

SIDILQEKEGHLDFVIIPHYTFL [symbol] YYKHLSY [symbol] [symbol] IYHKSSTYGKYIAVDAF
IKKINETYDKVKSKCNDIKNDLI [symbol] TIKKLEHPYDINNKNRAFKKMMDEYN
TKKKKLIKCIKNHENDFNKICMDMKNYGTNLFEQLSCYNNNFCNTNGIRY
HYDEYIHKLI [symbol] SVKSKNLNKDL [symbol] DMTNILQQSE [symbol] LLTNL [symbol] KKMGSYIYID
TIKFIHKEMKHIFNRIEYHTKIINDKTKIIQDKIKL [symbol] IWRTFQKDELLKR
ILDM [symbol] [symbol] EY [symbol] LF [symbol] TSD [symbol] LRQMLYNTFYSKEKHLNNIFHHLIYVLQMKFNDV
PI [symbol] MEYFQTYKKNKPLTQ

Modified residues compared with corresponding unmodified PfRH5 antigen are shown in bold and italicised.

PfRH5ΔNL_{HS3} Excluding Signal Sequence, Flexible N-Terminal (Amino Acids 1 to 139) and flexible Loop (Amino Acids 248 to 296) and comprising a Hexa-Histidine C-Terminal Tag (Underlined): SEQ ID NO: 51

KNVNFLQYHFKELSNYNIANSIDILQEKEGHLDFVIIPHYTFL [symbol] YYKHLS
Y [symbol] [symbol] IYHKSSTYGKYIAVDAFIKKINEAYDKVKSKCNDIKNDLI [symbol] TIKKLE
HPYDINNKNRAFKKMMDEYNTKKKKLIKCIKNHENDFNKICMDMKNYGTN
LFEQLSCYNNNFCNTNGIRYHYDEYIHKLI [symbol] SVKSKNLNKDL [symbol] DMTNILQ
QSE [symbol] LLTNL [symbol] KKMGSYIYIDTIKFIHKEMKHIFNRIEYHTKIINDKTKII
QDKIKL [symbol] IWRTFQKDELLKRILDM [symbol] [symbol] EY [symbol] LF [symbol] TSD [symbol] LRQMLYNTFYSKEK
HLNNIFHHLIYVLQMKFNDVPI [symbol] MEYFQTYKKNKPLTQHHHHHH

Modified residues compared with corresponding unmodified PfRH5 antigen are shown in bold and italicised.

PfRH5ΔNL_{HS3} Excluding Signal Sequence, Flexible N-Terminal (Amino Acids 1 to 159) and Flexible Loop (Amino Acids 248 to 296) and comprising a Hexa-Histidine C-Terminal Tag (Underlined): SEQ ID NO: 52

SIDILQEKEGHLDFVIIPHYTFL [symbol] YYKHLSY [symbol] [symbol] IYHKSSTYGKYIAVDAF
IKKINETYDKVKSKCNDIKNDLI [symbol] TIKKLEHPYDINNKNRAFKKMMDEYN
TKKKKLIKCIKNHENDFNKICMDMKNYGTNLFEQLSCYNNNFCNTNGIRY
HYDEYIHKLI [symbol] SVKSKNLNKDL [symbol] DMTNILQQSE [symbol] LLTNL [symbol] KKMGSYIYID
TIKFIHKEMKHIFNRIEYHTKIINDKTKIIQDKIKL [symbol] IWRTFQKDELLKR
ILDM [symbol] [symbol] EY [symbol] LF [symbol] TSD [symbol] LRQMLYNTFYSKEKHLNNIFHHLIYVLQMKFNDV
PI [symbol] MEYFQTYKKNKPLTQHHHHHH

Modified residues compared with corresponding unmodified PfRH5 antigen are shown in bold and italicised.

PfRH5ΔNL_{HS3} Excluding Signal Sequence, Flexible N-Terminal (Amino Acids 1 to 139) and Flexible Loop (Amino Acids 248 to 296) and comprising a Bip Leader Sequence (Dotted Underlined): SEQ ID NO: 53

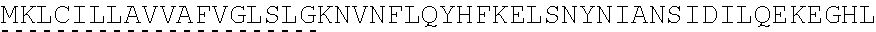

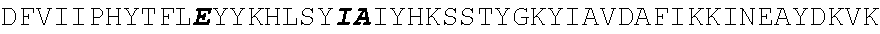

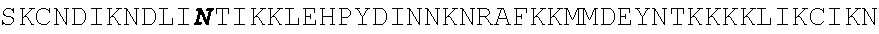

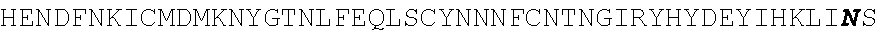

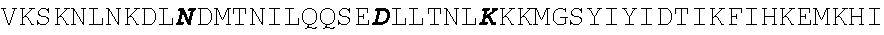

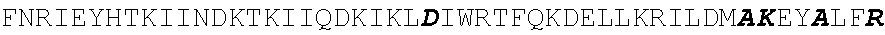

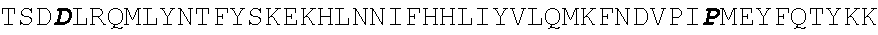

NKPLTQ

Modified residues compared with corresponding unmodified PfRH5 antigen are shown in bold and italicised.

PfRH5ΔNL_{HS3} Excluding Signal Sequence, Flexible N-Terminal (Amino Acids 1 to 159) and Flexible Loop (Amino Acids 248 to 296) and comprising a Bip Leader Sequence (Dotted Underlined): SEQ ID NO: 54

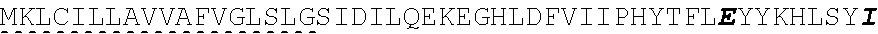

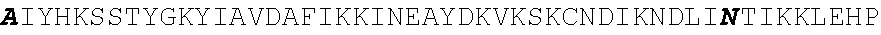

YDINNKNRAFKKMMDEYNYKKKKLIKCIKNHENDFNKICMDMKNYGTNLF

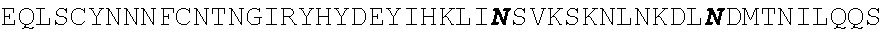

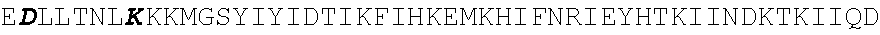

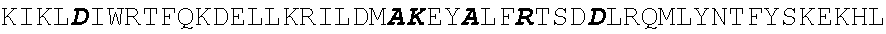

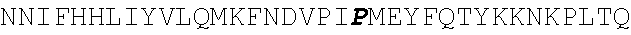

Modified residues compared with corresponding unmodified PfRH5 antigen are shown in bold and italicised.

PfRH5ΔNL_{HS3} Excluding Signal Sequence, Flexible N-Terminal (Amino Acids 1 to 139) and Flexible Loop (Amino Acids 248 to 296) and comprising a Bip Leader Sequence (Dotted Underlined) and a Hexa-Histidine C-Terminal Tag (Underlined): SEQ ID NO: 55

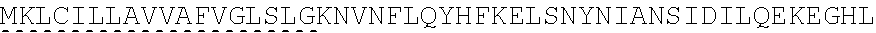

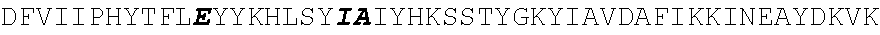

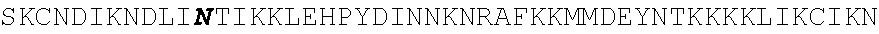

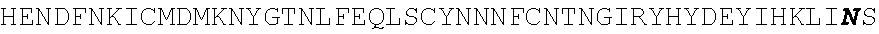

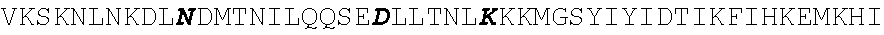

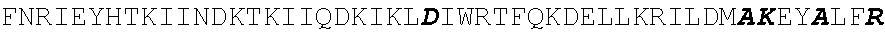

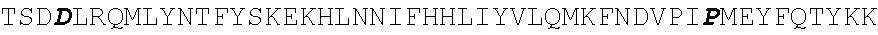

NKPLTQHHHHHH

Modified residues compared with corresponding unmodified PfRH5 antigen are shown in bold and italicised.

PfRH5ΔNL_{HS3} Excluding Signal Sequence, Flexible N-Terminal (Amino Acids 1 to 159) and Flexible Loop (Amino Acids 248 to 296) and comprising a Bip Leader Sequence (Dotted Underlined) and a Hexa-Histidine C-Terminal Tag (Underlined): SEQ ID NO: 56

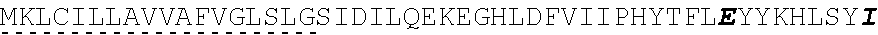

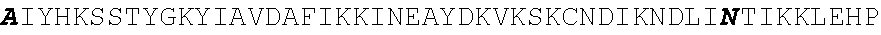

YDINNKNRAFKKMMDEYNTKKKKLIKCIKNHENDFNKICMDMKNYGTNLF

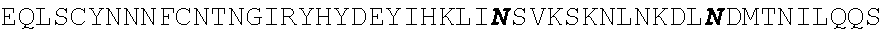

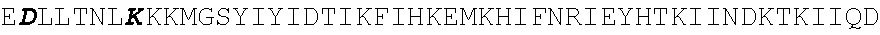

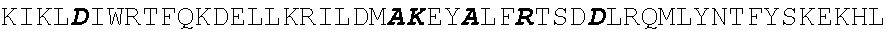

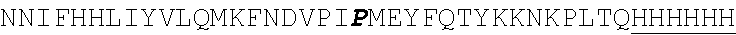

Modified residues compared with corresponding unmodified PfRH5 antigen are shown in bold and italicised.

1. A vaccine composition comprising a modified Reticulocyte-binding protein Homologue 5 (PfRH5) antigen, wherein said modified PfRH5 antigen comprises an amino acid substitution at five or more of amino acid positions 183, 233, 381, 392, 398, 464, 467, 57, 164, 171,178, 188, 191, 192, 195, 221, 230, 231, 234, 236, 300, 304, 305, 308, 309, 311, 312, 314, 315, 316, 330, 336, 354, 365, 368, 369, 370, 384, 390, 391, 394, 395, 396, 401, 406, 414, 422, 424, 428, 435, 442, 444, 445, 455, 458, 463, 468, 470, 474, 479, 481, 485, 495, 505 and/or 511, or any combination thereof, relative to the corresponding unmodified PfRH5 antigen.

2. The composition of claim 1, wherein said modified PfRH5 antigen comprises an amino acid substitution at each of positions 183, 233, 381, 392, 398, 464 and/or 467 relative to the corresponding unmodified PfRH5 antigen.

3. The composition of claim 1, wherein said modified PfRH5 antigen further comprises one or more amino acid substitution at position 157, 191, 192, 236, 304, 308, 312, 314, 316, 330, 369, 370, 384, 395, 414, 444, 445, 458, 463, 470, 474, 495, 505 and/or 511, or any combination thereof, relative to the corresponding unmodified PfRH5 antigen.

4. The composition of claim 1, wherein said modified PfRH5 antigen comprises amino acid substitutions at:
(a) positions 157, 183, 233, 304, 312, 314, 316, 330, 370, 381, 384, 392, 395, 398, 458, 464, 467 and 505 relative to the corresponding unmodified PfRH5 antigen;
(b) positions 183, 191, 192, 233, 369, 381, 392, 398, 445, 463, 464, 467, 470, 474 and 511 relative to the corresponding unmodified PfRH5 antigen; or
(c) positions 183, 191, 192, 233, 236, 308, 314, 369, 370, 381, 384, 392, 395, 398, 414, 444, 445, 463, 464, 467, 470, 474, 495, 505 and 511 relative to the corresponding unmodified PfRH5 antigen.

5. (canceled)

6. (canceled)

7. The composition of claim 1, wherein the amino acid at position:
(i) 157 is substituted by a leucine;
(ii) 183 is substituted by a glutamic acid;
(iii) 191 is substituted by an isoleucine;
(iv) 192 is substituted by an alanine;
(v) 233 is substituted by a lysine or asparagine;
(vi) 236 is substituted by a histidine;
(vii) 304 is substituted by a phenylalanine;
(viii) 308 is substituted by a lysine;
(ix) 312 is substituted by an asparagine;
(x) 314 is substituted by a phenylalanine;
(xi) 316 is substituted by an asparagine;
(xii) 330 is substituted by an asparagine;
(xiii) 369 is substituted by an asparagine;
(xiv) 370 is substituted by an alanine or lysine;
(xv) 381 is substituted by an asparagine;
(xvi) 384 is substituted by a lysine;
(xvii) 392 is substituted by a lysine or aspartic acid;
(xviii) 395 is substituted by an asparagine or arginine;
(xix) 398 is substituted by a glutamic acid or lysine;
(xx) 414 is substituted by a leucine;
(xxi) 444 is substituted by a glutamic acid;
(xxii) 445 is substituted by an aspartic acid;
(xxiii) 458 is substituted by a lysine;
(xxiv) 463 is substituted by an alanine;
(xxv) 464 is substituted by a lysine;
(xxvi) 467 is substituted by an alanine;
(xxvii) 470 is substituted by an arginine;
(xxviii) 474 is substituted by an aspartic acid;
(xxix) 495 is substituted by an asparagine;
(xxx) 505 is substituted by a leucine; and/or
(xxxi) 511 is substituted by a proline;
or any combination thereof.

8. The composition of claim 1, wherein the modified PfRH5 antigen comprises the following amino acid substitutions:
(a) I157L, D183E, A233K, M304F, K312N, L314F, K316N, M330N, S370A, S381N, T384K, L392K, T395N, N398E, R458K, N464K, S467A and F505L;
(b) D183E, N191I, S192A, A233N, L369N, S381N, T392D, N398K, N445D, S463A, N464K, S467A, I470R, H474D and K511P; or
(c) D183E, N191I, S192A, A233N, K236H, N308K, L314F, L369N, S370K, S381N, T384K, T392D, T395R, N398K, H414L, L444E, N445D, S463A, N464K, S467A, I470R, H474D, H495N, F505L and K511P.

9. The composition of claim 1, wherein one or more of amino acid positions 147, 149, 193, 194, 196, 197, 198, 200, 201, 202, 203, 204, 205, 206, 207, 209, 212, 213, 216, 222, 225, 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and/or 496, or any combination thereof, is unchanged in the modified PfRH5 antigen relative to the corresponding unmodified PfRH5 antigen.

10. The composition of claim 1, wherein all of amino acid positions 147, 149, 193, 194, 196, 197, 198, 200, 201, 202, 203, 204, 205, 206, 207, 209, 212, 213, 216, 222, 225, 226, 242, 243, 244, 245, 246, 247, 248, 249, 250, 327, 328, 331, 334, 335, 337, 338, 339, 340, 341, 342, 343, 344, 345, 346, 347, 348, 349, 350, 352, 353, 357, 358, 362, 447, 448, 449, 451, 452, 456 and 496 are unchanged relative in the modified PfRH5 antigen relative to the corresponding unmodified PfRH5 antigen.

11. (canceled)

12. The composition of claim 1, wherein the unmodified PfRH5 antigen is a basigin-binding fragment of PfRH5 comprising:
(a) amino acid residues 140 to 526 of SEQ ID NO: 1 or 2, or a fragment of an amino acid sequence having at least 90% sequence identity to amino acid residues 140 to 526 of SEQ ID NO: 1 or 2; or
(b) amino acid residues 160 to 526 of SEQ ID NO: 1 or 2, or a fragment of an amino acid sequence having at least 90% sequence identity to amino acid residues 160 to 526 of SEQ ID NO: 1 or 2.

13. The composition of claim 12, wherein said fragment of PfRH5 has the amino acid sequence of any one of SEQ ID NOs: 3 to 6, or an amino acid sequence having at least 90% sequence identity to one of SEQ ID NOs: 3 to 6.

14. The composition of claim 1, wherein the unmodified PfRH5 antigen is a discontinuous fragment of PfRH5, wherein optionally said discontinuous fragment of PfRH5 lacks the flexible loop region corresponding to amino acid residues 248 to 296 of SEQ ID NO: 1 or 2.

15. The composition of claim 14, wherein said discontinuous fragment of PfRH5 has at least 90% sequence identity to any one of SEQ ID NO: 7 to 14, preferably SEQ ID NO: 7 to 10.

16. The composition of claim 1, wherein the modified PfRH5 antigen comprises the amino acid sequence of any one of SEQ ID NOs: 15 to 56, preferably any one of SEQ ID NOs: 21 to 28, 35 to 42 or 49 to 56.

17. The composition of claim 1, which induces antibodies that have a growth inhibitory activity (GIA) of at least 50% at a concentration of 10 mg/ml.

18. The composition of claim 1, wherein the composition further comprises one or more antigens selected from PfAMA1, PfEBA175, PfRH1, PfRH2a, PfRH2b, PfRH4, PfCyRPA, PfRIPR, PfP113 and/or PfAARP, or a fragment thereof.

19. The composition of claim 1, wherein said modified PfRH5 antigen is in the form of a recombinant protein, a protein particle, a virus-like particle, a fusion protein, or a combination thereof.

20. The composition of claim 18, comprising a fusion of the modified PfRH5 antigen and one or more antigens selected from PfAMA1, PfEBA175, PfRH1, PfRH2a, PfRH2b, PfRH4, PfCyRPA, PfRIPR, PfP113 and/or PfAARP, or a fragment thereof.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A method of treating and/or preventing malaria in a subject, comprising administering to the subject a therapeutically effective amount of the vaccine composition of claim 1.

38. (canceled)

39. The method of claim 37, wherein the treatment and/or prevention comprises priming the subject with a human or simian adenovirus, and boosting the subject with a pox virus.

40. (canceled)

41. The method of claim 37 wherein the modified PfRH5 antigen results in antibodies with a growth inhibitory activity (GIA) of at least 50% against the blood-stage Plasmodium parasite.

42. (canceled)

43. (canceled)

44. (canceled)
